(19) United States
(12) Patent Application Publication
(10) Pub. No.: US 2014/0275207 A1
(43) Pub. Date: Sep. 18, 2014

(54) ANTISENSE COMPOUNDS AND METHODS OF USE THEREOF

(71) Applicant: ISIS PHARMACEUTICALS, INC., Carlsbad, CA (US)

(72) Inventors: Sanjay K. Pandey, Encinitas, CA (US); Robert McKay, Poway, CA (US); Sanjay Bhanot, Carlsbad, CA (US); Xing-Xian Yu, San Diego, CA (US)

(73) Assignee: ISIS PHARMACEUTICALS, INC., Carlsbad, CA (US)

(21) Appl. No.: 13/804,846

(22) Filed: Mar. 14, 2013

(51) Int. Cl. C07H 21/00

(52) U.S. Cl. CPC C07H 21/00
USPC 514/44 A; 536/24.5

(57) ABSTRACT

Disclosed herein are compounds, compositions and methods for modulating the expression of LMW-PTPase in a cell, tissue or animal. Also provided are methods of target validation. Also provided are uses of disclosed compounds and compositions in the manufacture of a medicament for treatment of diseases and disorders. Also provided are methods for the prevention, amelioration and/or treatment of diabetes, insulin resistance, insulin deficiency, hypercholesterolemia, hyperglycemia, dyslipidemia, hyperlipidemia, hypertriglyceridemia, and hyperfattyacidemia. In some embodiments, the diabetes is type II diabetes by administration of antisense compounds targeted to LMW-PTPase.

### SEQUENCE LISTING

The present application is being filed along with a Sequence Listing in electronic format. The Sequence Listing is provided as a file entitled ISISLMWPSEQ.txt, created on Mar. 13, 2013 which is 152 Kb in size. The information in the electronic format of the sequence listing is incorporated herein by reference in its entirety.

### BACKGROUND OF THE INVENTION

Considerable attention has been devoted to the characterization of tyrosine kinases and tyrosine phosphatases and their associations with disease states (Zhang, Crit. Rev. Biochem. Mol. Biol., 1998, 33, 1-52). LMW-PTPase [also known ACP1; Acid phosphatase 1, soluble; Bf isoform; Bs isoform; HAAP; HCPTP; Cytoplasmic Phosphotyrosyl Protein Phosphatase; MGC3499; RCAP; Red cell acid phosphatase 1, isozyme F; Red cell acid phosphatase 1, isozyme S; acid phosphatase of erythrocyte; adipocyte acid phosphatase; low molecular weight phosphotyrosine protein phosphatase; red cell acid phosphatase 1] was originally isolated as an acid phosphatase from red blood cells and was subsequently found to be expressed in many additional tissues, including placenta, brain, kidney, liver, and leukocytes (Bryson et al., Genomics, 1995, 30, 133-140; Dissing and Svensmark, Biochim Biophys. Acta., 1990, 1041, 232-242; Hopkinson et al., Nature, 1963, 199, 969-971; Wo et al., J. Biol. Chem., 1992, 267, 10856-10865). The LMW-PTPase locus was mapped to chromosome 2 at 2p23-p25 (Bryson et al., Genomics, 1995, 30, 133-140; Magenis et al., Birth. Defects Orig. Artie. Ser., 1976, 12, 326-327; Wakita et al., Hum. Genet., 1985, 71, 259-260) and found to contain seven exons spanning 18 kilobases (Emanuel et al., Am. J. Med. Genet., 1979, 4, 167-172; Junien et al., Hum. Genet., 1979, 48, 17-21).

LMW-PTPase interacts directly with insulin stimulated insulin receptors, and negatively modulates metabolic and mitogenic insulin signaling (Chiarugi et al., Biochem. Biophys. Res. Commun., 1997, 238, 676-682). A recombinant form of one LMW-PTPase isoforma, HAAPβ, dephosphorylates the adipocyte lipid binding protein (ALBP), which may be a substrate for insulin receptor kinase (Shekels et al., Protein Sci., 1992, 1, 710-721). Together, these findings suggest a role for LMW-PTPase in regulating insulin signaling. LMW-PTPase also modulates flavin mononucleotide (FMN) levels, and dephosphorylates Band 3, the erythrocyte anion transporter. These functions regulate red blood cell metabolism and integrity and account for the association between LMW-PTPase and diseases such as hemolytic favism, a disease characterized by an acute idiosyncratic hemolytic response to molecules derived from fava beans (Bottini et al., Arch. Immunol. Ther. Exp. (Warsz), 2002, 50, 95-104).

The most common genetic polymorphisms of LMW-PTPase (also known as ACP1) result in the occurrence of three alleles (ACP1*A, ACP1*B and ACP1*C) and account for six different genotypes, each of which exhibits strong variations in total enzymatic activity (Golden and Sensabaugh, Hum. Genet., 1986, 72, 340-343; Hopkinson et al., Nature, 1963, 199, 969-971). Numerous rare alleles have been reported, including ACP1*D, E, F, G, H, I, K, M, R, TIC1, GUA, and a silent allele, ACP1*Q0 (Miller et al., Hum. Hered., 1987, 37, 371-375). Alternative splicing accounts for two isoforms which have been labeled fast (F) and slow (S), based on their electrophoretic mobility (Dissing, Biochem. Genet., 1987, 25, 901-918). The F and S isoforms exhibit different enzymatic properties, which, coupled with differences in the ratios of these isozymes, results in variations in activity modulation (Bottini et al., Hum. Genet., 1995, 96, 629-637).

Protein tyrosine phosphatases are signaling molecules that regulate a variety of cellular processes, including cell growth and differentiation, cell cycle progression and growth factor signaling. For example, a number of protein tyrosine phosphatases have been implicated as negative regulators of insulin signaling (Zhang, Crit. Rev. Biochem. Mol. Biol., 1998, 33, 1-52). LMW-PTPase is a phosphotyrosine phosphatase that is involved in multiple signal transduction pathways. For example, LMW-PTPase interacts directly with insulin stimulated insulin receptors, and negatively modulates metabolic and mitogenic insulin signaling (Chiarugi et al., Biochem. Biophys. Res. Commun., 1997, 238, 676-682). A recombinant form of one LMW-PTPase isoforma, HAAPβ, dephosphorylates the adipocyte lipid binding protein (ALBP), which may be a substrate for insulin receptor kinase (Shekels et al., Protein Sci., 1992, 1, 710-721). Together, these findings suggest a role for LMW-PTPase in regulating insulin signaling. LMW-PTPase also modulates flavin mononucleotide (FMN) levels, and dephosphorylates Band 3, the erythrocyte anion transporter. These functions regulate red blood cell metabolism and integrity and account for the association between LMW-PTPase and diseases such as hemolytic favism, a disease characterized by an acute idiosyncratic hemolytic response to molecules derived from fava beans (Bottini et al., Arch. Immunol Ther. Exp. (Warsz), 2002, 50, 95-104).

The most common genetic polymorphisms of LMW-PTPase (also known as ACP1) result in the occurrence of three alleles (ACP1*A, ACP1*B and ACP1*C) and account for six different genotypes, each of which exhibits strong variations in total enzymatic activity (Golden and Sensabaugh, Hum. Genet., 1986, 72, 340-343; Hopkinson et al., Nature, 1963, 199, 969-971). Numerous rare alleles have been reported, including ACP1*D, E, F, G, H, I, K, M, R, TIC1, GUA, and a silent allele, ACP1*Q0 (Miller et al., Hum. Hered., 1987, 37, 371-375). Alternative splicing accounts for two isoforms which have been labeled fast (F) and slow (S), based on their electrophoretic mobility (Dissing, Biochem. Genet., 1987, 25, 901-918). The F and S isoforms exhibit different enzymatic properties, which, coupled with differences in the ratios of these isozymes, results in variations in activity modulation (Bottini et al., Hum. Genet., 1995, 96, 629-637). LMW-PTPase genotypes, and consequently isoform levels and total enzymatic activity, show correlation to a number of disease states. (See, e.g., Bottini et al., Arch. Immunol. Ther. Exp. (Warsz), 2002, 50, 95-104). These findings, together with the evidence that LMW-PTPase participates in insulin signaling, support a role for LMW-PTPase in metabolic disorders such as diabetes.

Given the genetic evidence for the involvement of LMW-PTPase in human disease, pharmacological modulation of LMW-PTPase activity and/or expression is an appropriate point of therapeutic intervention in these and other pathological conditions. Currently, there are no known therapeutic agents which effectively inhibit the synthesis of LMW-PTPase. Consequently, there remains a long felt need for agents capable of effectively inhibiting LMW-PTPase function.

Antisense technology is an effective means for reducing the expression of LMW-PTPase and is uniquely useful in a number of therapeutic, diagnostic, and research applications. Generally, the principle behind antisense technology is that an antisense compound hybridizes to a target nucleic acid and effects the modulation of gene expression activity, or function, such as transcription or translation. The modulation of gene expression can be achieved by, for example, target RNA degradation or occupancy-based inhibition. An example of modulation of target RNA function by degradation is RNase H-based degradation of the target RNA upon hybridization with a DNA-like antisense compound. Another example of modulation of gene expression by target degradation is RNA interference (RNAi) using small interfering RNAs (siRNAs). RNAi is a form of antisense-mediated gene silencing involving the introduction of double stranded (ds)RNA-like oligonucleotides leading to the sequence-specific reduction of targeted endogenous mRNA levels. This sequence-specificity makes antisense compounds extremely attractive as tools for target validation and gene functionalization, as well as therapeutics to selectively modulate the expression of genes involved in diseases.

### SUMMARY OF THE INVENTION

Disclosed herein are antisense compounds targeted to and hybridizable with a nucleic acid molecule encoding LMW-PTPase and which modulate the expression of LMW-PTPase. In a preferred embodiment the nucleic acid molecule encoding LMW-PTPase has a nucleotide sequence that is substantially similar to one or more of GenBank Accession Nos.: NM_{—}004300.2, NM_{—}007099.2, NM_{—}177554.1, and NT_{—}022327.13_(SEQ ID NOS: 3-6, respectively), presented in table 1, below and incorporated herein by reference. In a further aspect, the antisense compounds are targeted to and hybridizable with a region of a nucleic acid molecule encoding LMW-PTPase. Still further, the antisense compounds are targeted to and hybridizable with a segment of a nucleic acid molecule encoding LMW-PTPase. Still further the antisense compounds are targeted to and hybridizable with a site of a nucleic acid molecule encoding LMW-PTPase.

Further disclosed herein are active target segments comprising segments of a nucleic acid molecule encoding LMW-PTPase, the active target segments being accessible to antisense hybridization, and so, suitable for antisense modulation. In one embodiment, the active target segments have been discovered herein using empirical data that is presented below, wherein at least two chimeric oligonucleotides are shown to hybridize within the active target segment and reduce expression of the target nucleic acid (hereinafter, “active antisense compound”). The at least two active antisense compounds are preferably separated by about 60 nucleobases on the nucleic acid molecule encoding LMW-PTPase. In another embodiment, antisense compounds are designed to target the active target segments and modulate expression of the nucleic acid molecule encoding LMW-PTPase.

In one aspect there are herein provided antisense compounds comprising sequences 12 to 35 nucleotides in length comprising at least two chemical modifications selected from a modified internucleoside linkage, a modified nucleobase or a modified sugar. Provided herein are chimeric oligonucleotides comprising a deoxynucleotide mid-region flanked on each of the 5′ and 3′ ends by wing regions, each wing region comprising at least one high affinity nucleotide.

In one embodiment there is herein provided chimeric oligonucleotides comprising ten deoxynucleotide mid-regions flanked on each of the 5′ and 3′ ends with wing regions comprising five 2′-O-(2-methoxyethyl) nucleotides and wherein each internucleoside linkage of the chimeric oligonucleotid is a phosphorothioate. In another embodiment there is herein provided chimeric oligonucleotides comprising fourteen deoxynucleotide mid-regions flanked on each of the 5′ and 3′ ends with wing regions comprising three locked nucleic acid nucleotides and wherein each internucleoside linkage of the chimeric oligonucleotide is a phosphorothioate. In a further embodiment there are hererin provided chimeric oligonucleotides comprising fourteen deoxynucleotide mid-regions flanked on each of the 5′ and 3′ ends by wing regions comprising two 2′-O-(2-methoxyethyl) nucleotides and wherein each internucleoside linkage of the chimeric oligonucleotide is a phosphorothioate. In a further embodiment, the antisense compounds may comprise at least one 5-methylcytosine.

Further provided are methods of modulating the expression of LMW-PTPase in cells, tissues or animals comprising contacting said cells, tissues or animals with one or more of the compounds or compositions of the present invention. For example, in one embodiment, the compounds can be used to inhibit the expression of LMW-PTPase in cells, tissues or animals. In this aspect of the invention cells are analyzed for indicators of a decrease in expression of LMW-PTPase mRNA and/or protein by direct measurement of mRNA and/or protein levels, and/or indicators of a disease or condition, such as glucose levels, lipid levels, weight, or a combination thereof.

One embodiment provides methods of lowering glucose and triglycerides. Glucose may be blood, plasma or serum glucose. Triglycerides may be blood, plasma, or serum triglycerides. Another embodiment provides methods of improving insulin sensitivity. Another embodiment provides methods of lowering cholesterol. In some embodiments, cholesterol is LDL or VLDL cholesterol. An embodiment provides methods of improving glucose tolerance.

Other embodiments are directed to methods of ameliorating or lessening the severity of a condition in an animal comprising contacting said animal with an effective amount of an antisense compound so that expression of LMW-PTPase is inhibited and measurement of one or more physical indicator of said condition indicates a lessening of the severity of said condition. In some embodiments, the conditions include, but are not limited to, diabetes, insulin resistance, insulin deficiency, hypercholesterolemia, hyperglycemia, dyslipidemia, hyperlipidemia, hypertriglyceridemia, and hyperfattyacidemia. In some embodiments, the diabetes is type II diabetes. In another embodiment, the condition is metabolic syndrome. In another embodiment, the condition is prediabetes. In another embodiment the condition is steatosis. In one embodiment, the steatosis is steatohepatitis. In another embodiment, the steatosis is NASH. In another embodiment, the condition is a cardiovascular disease. In another embodiment, the cardiovascular disease is coronary heart disease. In another embodiment, the condition is a cardiovascular risk factor.

In another embodiment, there is provided a method of decreasing hepatic glucose output in an animal comprising administering an oligomeric compound of the invention. In one embodiment, the present invention provides a method of decreasing hepatic glucose-6-phosphatase expression comprising administering an oligomeric compound of the invention. Another aspect of the present invention is a method of reducing LMW-PTPase expression in liver, fat, or in both tissues.

Also provided are methods of ameliorating or lessening the severity of a condition in an animal comprising contacting said animal with an oligomeric compound of the invention in combination with a glucose-lowering, lipid-lowering, or anti-obesity agent to achieve an additive therapeutic effect.

Also provided are methods for the prevention, amelioration, and/or treatment of diabetes, type II diabetes, prediabetes, insulin resistance, insulin deficiency, hypercholesterolemia, hyperglycemia, dyslipidemia, hyperlipidemia, hypertriglyceridemia, metabolic syndrome, hyperfattyacidemia, steatosis, steatohepatitis, NASH, cardiovascular disease, coronary heart disease, a cardiovascular risk factor or combinations thereof comprising administering at least one compound of the instant invention to an individual in need of such intervention.

The invention also provides a method of use of the compositions of the instant invention for the preparation of a medicament for the prevention, amelioration, and/or treatment disease, especially a disease associated with and including at least one indicator of diabetes, type II diabetes, prediabetes, insulin resistance, insulin deficiency, hypercholesterolemia, hyperglycemia, dyslipidemia, hyperlipidemia, hypertriglyceridemia, metabolic syndrome, hyperfattyacidemia, steatosis, steatohepatitis, NASH, cardiovascular disease, coronary heart disease, a cardiovascular risk factor or combinations thereof.

### DETAILED DESCRIPTION OF THE INVENTION

LMW-PTPase is shown to effect in vivo glucose levels, triglyceride levels, cholesterol levels, insulin sensitivity and glucose tolerance, therefore, LMW-PTPase is indicated in diseases and conditions related thereto and including, but not limited to, diabetes, type II diabetes, obesity, insulin resistance, insulin deficiency, hypercholesterolemia, hyperglycemia, hyperlipidemia, hypertriglyceridemia, hyperfattyacidemia, liver steatosis, steatohepatitis, non-alcoholic steatohepatitis, metabolic syndrome, cardiovascular disease and coronary heart disease. Provided herein are antisense compounds for the prevention, amelioration, and/or treatment of diseases and conditions relating to LMW-PTPase function. As used herein, the term “prevention” means to delay or forestall onset or development of a condition or disease for a period of time from hours to days, preferably weeks to months. As used herein, the term “amelioration” means a lessening of at least one indicator of the severity of a condition or disease. The severity of indicators may be determined by subjective or objective measures which are known to those skilled in the art. As used herein, “treatment” means to administer a composition of the invention to effect an alteration or improvement of the disease or condition. Prevention, amelioration, and/or treatment may require administration of multiple doses at regular intervals, or prior to exposure to an agent to alter the course of the condition or disease.

Disclosed herein are antisense compounds, including antisense oligonucleotides and other antisense compounds for use in modulating the expression of nucleic acid molecules encoding LMW-PTPase. This is accomplished by providing antisense compounds that hybridize with one or more target nucleic acid molecules encoding LMW-PTPase. As used herein, the terms “target nucleic acid” and “nucleic acid molecule encoding LMW-PTPase” have been used for convenience to encompass RNA (including pre-mRNA and mRNA or portions thereof) transcribed from DNA encoding LMW-PTPase, and also cDNA derived from such RNA. In a preferred embodiment, the target nucleic acid is an mRNA encoding LMW-PTPase.

### Target Nucleic Acids

“Targeting” an antisense compound to a particular target nucleic acid molecule can be a multistep process. The process usually begins with the identification of a target nucleic acid whose expression is to be modulated. For example, the target nucleic acid can be a cellular gene (or mRNA transcribed from the gene) whose expression is associated with a particular disorder or disease state, or a nucleic acid molecule from an infectious agent. As disclosed herein, the target nucleic acid encodes LMW-PTPase and has a polynucleotide sequence that is substantially similar to one or more of SEQ ID NOS: 1-4.

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequence. Variants can result in mRNA variants including, but not limited to, those with alternate splice junctions, or alternate initiation and termination codons. Variants in genomic and mRNA sequences can result in disease. Antisense compounds targeted to such variants are within the scope of the instant invention.

In accordance with the present invention are compositions and methods for modulating the expression of LMW-PTPase. Table 1 lists the GenBank accession numbers of sequences corresponding to nucleic acid molecules encoding LMW-PTPase (nt=nucleotide), the date the version of the sequence was entered in GenBank, and the corresponding SEQ ID NO in the instant application, when assigned, each of which is incorporated herein by reference.

TABLE 1
Gene Targets

| | | | SEQ |
|---|---|---|---|
| Species | Genbank # | Genbank Date | ID NO |
| Human | M83653.1 | Apr. 27, 1993 | 1 |
| Human | M83654.1 | Apr. 27, 1993 | 2 |
| Human | NM_004300.2 | Apr. 24, 2003 | 3 |
| Human | NM_007099.2 | Apr. 24, 2003 | 4 |
| Human | NM_177554.1 | Apr. 24, 2003* | 5 |
| Human | nucleotides 254496 to 268683 | Oct. 7, 2003 | 6 |
| | of NT_022327.13 | | |
| Human | U25847.1 | Jan. 5, 1996 | 7 |
| Human | U25848.1 | Jan. 5, 1996 | 8 |
| Human | U25849.1 | Jan. 5, 1996 | 9 |
| Human | Y16846.1 | Jun. 16, 1998 | 10 |
| Mouse | BF167197.1 | Oct. 27, 2000 | 11 |
| Mouse | NM_021330.1 | Oct. 23, 2000 | 12 |
| Mouse | the complement of nucleotides | Oct. 30, 2003 | 13 |
| | 6757610 to 6776640 of | | |
| | NT_039548.2 | | |
| Mouse | Y17343.1 | Jul. 8, 1998 | 14 |
| Mouse | Y17344.1 | Jul. 8, 1998 | 355 |
| Mouse | Y17345.1 | Jul. 8, 1998 | 15 |
| Rat | NM_021262.2 | Jan. 1, 2004 | 16 |
| Rat | the complement of nucleotides | Sep. 22, 2003 | 17 |
| | 905000 to 921000 of | | |
| | NW_047759.1 | | |
| Rat | XM_343044.1 | Sep. 22, 2003 | 18 |

*NM_177554.1 was permanently suppressed because it is a nonsense-mediated mRNA decay (NMD) candidate.

### Modulation of Target Expression

Modulation of expression of a target nucleic acid can be achieved through alteration of any number of nucleic acid (DNA or RNA) functions. “Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of LMW-PTPase. Thus, in one embodiment modulation of expression can mean increase or decrease in target RNA or protein levels. In another embodiment modulation of expression can mean an increase or decrease of one or more RNA splice products, or a change in the ratio of two or more splice products.

The effect of antisense compounds of the present invention on target nucleic acid expression can be tested in any of a variety of cell types provided that the target nucleic acid is present at measurable levels. The effect of antisense compounds of the present invention on target nucleic acid expression can be routinely determined using, for example, PCR or Northern blot analysis. Cell lines are derived from both normal tissues and cell types and from cells associated with various disorders (e.g. hyperproliferative disorders). Cell lines derived from multiple tissues and species can be obtained from American Type Culture Collection (ATCC, Manassas, Va.) and other public sources, and are well known to those skilled in the art. Primary cells, or those cells which are isolated from an animal and not subjected to continuous culture, can be prepared according to methods known in the art, or obtained from various commercial suppliers. Additionally, primary cells include those obtained from donor human subjects in a clinical setting (i.e. blood donors, surgical patients). Primary cells prepared by methods known in the art.

### Assaying Modulation of Expression

Modulation of LMW-PTPase expression can be assayed in a variety of ways known in the art. LMW-PTPase mRNA levels can be quantitated by, e.g., Northern blot analysis, competitive polymerase chain reaction (PCR), or real-time PCR. RNA analysis can be performed on total cellular RNA or poly(A)+ mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions. The method of analysis of modulation of RNA levels is not a limitation of the instant invention.

Levels of a protein encoded by LMW-PTPase can be quantitated in a variety of ways well known in the art, such as immunoprecipitation, Western blot analysis (immunoblotting), ELISA or fluorescence-activated cell sorting (FACS). Antibodies directed to a protein encoded by LMW-PTPase can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997.

### Active Target Segments

The locations on the target nucleic acid defined by having at least two active antisense compounds targeted thereto are referred to as “active target segments.” An active target segment is defined by one of the at least two active antisense compounds hybridizing at the 5′ end of the active target segment and the other hybridizing at the 3′ end of the active target segment. Additional active antisense compounds may hybridize within this defined active target segment. The compounds are preferably separated by no more than about 60 nucleotides on the target sequence, more preferably no more than about 30 nucleotides on the target sequence, even more preferably the compounds are contiguous, most preferably the compounds are overlapping. There may be substantial variation in activity (e.g., as defined by percent inhibition) of the antisense compounds within an active target segment. Active antisense compounds are those that modulate the expression of their target RNA. In one of the assays provided herein, active antisense compounds inhibit expression of their target RNA at least 10%, preferably 20%. In a preferred embodiment, at least about 50%, preferably about 70% of the oligonucleotides targeted to the active target segment modulate expression of their target RNA at least 40%. In a more preferred embodiment, the level of inhibition required to define an active antisense compound is defined based on the results from the screen used to define the active target segments. One ordinarily skilled in the art will readily understand that values received from any single assay will vary in comparison to other similar assays due to assay-to-assay conditions.

### Hybridization

As used herein, “hybridization” means the pairing of complementary strands of antisense compounds to their target sequence. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is complementary to the natural nucleobases thymidine and uracil which pair through the formation of hydrogen bonds. The natural base guanine is complementary to the natural base 5-methyl cytosine and the artificial base known as a G-clamp. Hybridization can occur under varying circumstances.

An antisense compound is specifically hybridizable when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

### Complementarity

“Complementarity,” as used herein, refers to the capacity for precise pairing between two nucleobases on either two oligomeric compound strands or an antisense compound with its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position. The antisense compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the antisense compound and a target nucleic acid.

Those in the art understand that for an antisense compound to be active it need not be 100% complementary to the target nucleic acid site wherein it hybridizes. Often, once an antisense compound has been identified as an active antisense compound, the compounds are routinely modified to include mismatched nucleobases compared to the sequence of the target nucleic acid site. The art teaches methods for introducing mismatches into an antisense compound without substantially altering its activity. Antisense compounds may be able to tolerate up to about 20% mismatches without significant alteration of activity, particularly so when a high affinity modification accompanies the mismatches.

### Identity

Antisense compounds, or a portion thereof, may have a defined percent identity to a SEQ ID NO, or a compound having a specific compound number. As used herein, a sequence is identical to the sequence disclosed herein if it has the same nucleobase pairing ability. For example, a RNA which contains uracil in place of thymidine in the disclosed sequences of the instant invention would be considered identical as they both pair with adenine. Similarly, a G-clamp modified heterocyclic base would be considered identical to a cytosine or a 5-Me cytosine in the sequences of the instant application as it pairs with a guanine. This identity may be over the entire length of the oligomeric compound, or in a portion of the antisense compound (e.g., nucleobases 1-20 of a 27-mer may be compared to a 20-mer to determine percent identity of the oligomeric compound to the SEQ ID NO.) It is understood by those skilled in the art that an antisense compound need not have an identical sequence to those described herein to function similarly to the antisense compound described herein. Shortened versions of antisense compound taught herein, or non-identical versions of the antisense compound taught herein fall within the scope of the invention. Non-identical versions are those wherein each base does not have the same pairing activity as the antisense compounds disclosed herein. Bases do not have the same pairing activity by being shorter or having at least one abasic site. Alternatively, a non-identical version can include at least one base replaced with a different base with different pairing activity (e.g., G can be replaced by C, A, or T). Percent identity is calculated according to the number of bases that have identical base pairing corresponding to the SEQ ID NO or antisense compound to which it is being compared. The non-identical bases may be adjacent to each other, dispersed through out the oligonucleotide, or both.

For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art.

The percent identity is based on the percent of nucleobases in the original sequence present in a portion of the modified sequence. Therefore, a 30 nucleobase antisense compound comprising the full sequence of the complement of a 20 nucleobase active target segment would have a portion of 100% identity with the complement of the 20 nucleobase active target segment, while further comprising an additional 10 nucleobase portion. In the context of the invention, the complement of an active target segment may constitute a single portion. In a preferred embodiment, the oligonucleotides of the instant invention are at least about 80%, more preferably at least about 85%, even more preferably at least about 90%, most preferably at least 95% identical to at least a portion of the complement of the active target segments presented herein.

It is well known by those skilled in the art that it is possible to increase or decrease the length of an antisense compound and/or introduce mismatch bases without eliminating activity. For example, in Woolf et al. (Proc. Natl. Acad. Sci. USA 89:7305-7309, 1992, incorporated herein by reference), a series of ASOs 13-25 nucleobases in length were tested for their ability to induce cleavage of a target RNA in an oocyte injection model. ASOs 25 nucleobases in length with 8 or 11 mismatch bases near the ends of the ASOs were able to direct specific cleavage of the target mRNA, albeit to a lesser extent than the ASOs that contained no mismatches. Similarly, target specific cleavage was achieved using a 13 nucleobase ASOs, including those with 1 or 3 mismatches. Maher and Dolnick (Nuc. Acid. Res. 16:3341-3358, 1988, incorporated herein by reference) tested a series of tandem 14 nucleobase ASOs, and a 28 and 42 nucleobase ASOs comprised of the sequence of two or three of the tandem ASOs, respectively, for their ability to arrest translation of human DHFR in a rabbit reticulocyte assay. Each of the three 14 nucleobase ASOs alone were able to inhibit translation, albeit at a more modest level than the 28 or 42 nucleobase ASOs.

### Therapeutics

Antisense compounds of the invention can be used to modulate the expression of LMW-PTPase in an animal, such as a human. In one non-limiting embodiment, the methods comprise the step of administering to said animal in need of therapy for a disease or condition associated with LMW-PTPase an effective amount of an antisense compound that inhibits expression of LMW-PTPase. A disease or condition associated with LMW-PTPase includes, but is not limited to, diabetes, type II diabetes, obesity, insulin resistance, insulin deficiency, hypercholesterolemia, hyperglycemia, hyperlipidemia, hypertriglyceridemia, hyperfattyacidemia, liver steatosis, steatohepatitis, non-alcoholic steatohepatitis, metabolic syndrome, cardiovascular disease and coronary heart disease. The diseases or conditions are associated with clinical indicators that include, but are not limited to blood glucose levels, blood lipid levels, hepatic lipid levels, insulin levels, cholesterol levels, transaminase levels, electrocardiogram, glucose uptake, gluconeogenesis, insulin sensitivity, body weight and combinations thereof. In one embodiment, the antisense compounds of the present invention effectively inhibit the levels or function of LMW-PTPase RNA. Because reduction in LMW-PTPase mRNA levels can lead to alteration in LMW-PTPase protein products of expression as well, such resultant alterations can also be measured. Antisense compounds of the present invention that effectively inhibit the level or function of LMW-PTPase RNA or protein products of expression are considered an active antisense compounds. In one embodiment, the antisense compounds of the invention inhibit the expression of LMW-PTPase causing a reduction of RNA by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of LMW-PTPase can be measured in a bodily fluid, tissue or organ of the animal. Methods of obtaining samples for analysis, such as body fluids (e.g., blood), tissues (e.g., biopsy), or organs, and methods of preparation of the samples to allow for analysis are well known to those skilled in the art. Methods for analysis of RNA and protein levels are discussed above and are well known to those skilled in the art. The effects of treatment can be assessed by measuring biomarkers associated with the LMW-PTPase expression in the aforementioned fluids, tissues or organs, collected from an animal contacted with one or more compounds of the invention, by routine clinical methods known in the art. These biomarkers include but are not limited to: liver transaminases, bilirubin, albumin, blood urea nitrogen, creatine and other markers of kidney and liver function; glucose levels, triglyceride levels, insulin levels, fatty acid levels, cholesterol levels, electrocardiogram, glucose uptake, gloconeogenesis, insulin sensitivity and body weight, and other markers of diabetes, type II diabetes, obesity, insulin resistance, insulin deficiency, hypercholesterolemia, hyperglycemia, hyperlipidemia, hypertriglyceridemia, hyperfattyacidemia, liver steatosis, steatohepatitis, non-alcoholic steatohepatitis, metabolic syndrome, cardiovascular disease and coronary heart disease. Additionally, the effects of reatment can be assessed using non-invasive indicators of improved disease state or condition, such as electrocardiogram, body weight, and the like.

The antisense compounds of the present invention can be utilized in pharmaceutical compositions by adding an effective amount of a compound to a suitable pharmaceutically acceptable diluent or carrier. Acceptable carriers and dilutents are well known to those skilled in the art. Selection of a dilutent or carrier is based on a number of factors, including, but not limited to, the solubility of the compound and the route of administration. Such considerations are well understood by those skilled in the art. In one aspect, the compounds of the present invention inhibit the expression of LMW-PTPase. The compounds of the invention can also be used in the manufacture of a medicament for the treatment of diseases and disorders related to LMW-PTPase expression by restoring glucose levels, triglyceride levels, insulin levels, fatty acid levels, cholesterol levels, glucose uptake, gloconeogenesis and insulin sensitivity to non-disease state profiles.

Methods whereby bodily fluids, organs or tissues are contacted with an effective amount of one or more of the antisense compounds or compositions of the invention are also contemplated. Bodily fluids, organs or tissues can be contacted with one or more of the compounds of the invention resulting in modulation of LMW-PTPase expression in the cells of bodily fluids, organs or tissues.

### Kits, Research Reagents, and Diagnostics

The antisense compounds of the present invention can be utilized for diagnostics, and as research reagents and kits. Furthermore, antisense compounds, which are able to inhibit gene expression with specificity, are often used by those of ordinary skill to elucidate the function of particular genes or to distinguish between functions of various members of a biological pathway.

For use in kits and diagnostics, the antisense compounds of the present invention, either alone or in combination with other compounds or therapeutics, can be used as tools in differential and/or combinatorial analyses to elucidate expression patterns of a portion or the entire complement of genes expressed within cells and tissues. Methods of gene expression analysis are well known to those skilled in the art.

### Antisense Compounds

The term “antisense compound” refers to a polymeric structure capable of hybridizing to a region of a nucleic acid molecule. As is used herein, the term “active antisense compound” is an antisense compound that has been shown to hybridize with the target nucleic acid and modulate it expression. Generally, antisense compounds comprise a plurality of monomeric subunits linked together by internucleoside linking groups and/or internucleoside linkage mimetics. Each of the monomeric subunits comprises a sugar, abasic sugar, modified sugar, or a sugar mimetic, and except for the abasic sugar includes a nucleobase, modified nucleobase or a nucleobase mimetic. Preferred monomeric subunits comprise nucleosides and modified nucleosides. An antisense compound is at least partially complementary to the region of a target nucleic acid molecule to which it hybridizes and which modulates (increases or decreases) its expression. This term includes oligonucleotides, oligonucleosides, oligonucleotide analogs, oligonucleotide mimetics, antisense compounds, antisense oligomeric compounds, and chimeric combinations of these. An “antisense oligonucleotide” is an antisense compound that is a nucleic acid-based oligomer. An antisense oligonucleotide can, in some cases, include one or more chemical modifications to the sugar, base, and/or internucleoside linkages. Nonlimiting examples of antisense compounds include antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. In some embodiments it is desirous to take advantage of alternate antisense mechanisms (such as RNAi). Antisense compounds that use these alternate mechanisms may optionally comprise a second compound which is complementary to the antisense compound. In other words, antisense double-stranded compounds can be two strands hybridized to form double-stranded compounds or a single strand with sufficient self complementarity to allow for hybridization and formation of a fully or partially double-stranded compound. The compounds of the instant invention are not auto-catalytic. As used herein, “auto-catalytic” means a compound has the ability to promote cleavage of the target RNA in the absence of accessory factors, e.g. proteins.

In one embodiment of the invention, double-stranded antisense compounds encompass short interfering RNAs (siRNAs). As used herein, the term “siRNA” is defined as a double-stranded compound having a first and second strand, each strand having a central portion and two independent terminal portions. The central portion of the first strand is complementary to the central portion of the second strand, allowing hybridization of the strands. The terminal portions are independently, optionally complementary to the corresponding terminal portion of the complementary strand. The ends of the strands may be modified by the addition of one or more natural or modified nucleobases to form an overhang

Each strand of the siRNA duplex may be from about 12 to about 35 nucleobases. In a preferred embodiment, each strand of the siRNA duplex is about 17 to about 25 nucleobases. The two strands may be fully complementary (i.e., form a blunt ended compound), or include a 5′ or 3′ overhang on one or both strands. Double-stranded compounds can be made to include chemical modifications as discussed herein.

In one embodiment of the invention, the antisense compound comprises a single stranded oligonucleotide. In some embodiments of the invention the antisense compound contains chemical modifications. In a preferred embodiment, the antisense compound is a single stranded, chimeric oligonucleotide wherein the modifications of sugars, bases, and internucleoside linkages are independently selected.

The antisense compounds may comprise a length from about 12 to about 35 nucleobases (i.e. from about 12 to about 35 linked nucleosides). In other words, a single-stranded compound of the invention comprises from about 12 to about 35 nucleobases, and a double-stranded antisense compound of the invention (such as a siRNA, for example) comprises two strands, each of which is independently from about 12 to about 35 nucleobases. This includes oligonucleotides 15 to 35 and 16 to 35 nucleobases in length. Contained within the antisense compounds of the invention (whether single or double stranded and on at least one strand) are antisense portions. The “antisense portion” is that part of the antisense compound that is designed to work by one of the aforementioned antisense mechanisms. One of ordinary skill in the art will appreciate that about 12 to about 35 nucleobases includes 12, 13, 14, 15, 16, 17, 18, 19, 20, 21, 22, 23, 24, 25, 26, 27, 28, 29, 30, 31, 32, 33, 34, or 35 nucleobases. For convenience we describe antisense compounds, but one ordinarily skilled in the art will understand that analogues and mimetics can have a length within this same range.

Antisense compounds about 12 to 35 nucleobases in length, preferably about 15 to 35 nucleobases in length, comprising a stretch of at least eight (8), preferably at least 12, more preferably at least 15 consecutive nucleobases selected from within the active target regions are considered to be suitable antisense compounds as well.

Modifications can be made to the antisense compounds of the instant invention and may include conjugate groups attached to one of the termini, selected nucleobase positions, sugar positions or to one of the internucleoside linkages. Possible modifications include, but are not limited to, 2′-fluoro (2′-F), 2′-OMethyl (2′-OMe), 2′-Methoxy ethoxy (2′-MOE) sugar modifications, inverted abasic caps, deoxynucleobases, and bicyclice nucleobase analogs such as locked nucleic acids (LNA.sup.™) and ENA.

### Chemical Modifications

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage. It is often preferable to include chemical modifications in oligonucleotides to alter their activity. Chemical modifications can alter oligonucleotide activity by, for example: increasing affinity of an antisense oligonucleotide for its target RNA, increasing nuclease resistance, and/or altering the pharmacokinetics of the oligonucleotide. The use of chemistries that increase the affinity of an oligonucleotide for its target can allow for the use of shorter oligonucleotide compounds.

The term “nucleobase” or “heterocyclic base moiety” as used herein, refers to the heterocyclic base portion of a nucleoside. In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable to the present invention. The terms modified nucleobase and nucleobase mimetic can overlap but generally a modified nucleobase refers to a nucleobase that is fairly similar in structure to the parent nucleobase, such as for example a 7-deaza purine or a 5-methyl cytosine, whereas a nucleobase mimetic would include more complicated structures, such as for example a tricyclic phenoxazine nucleobase mimetic. Methods for preparation of the above noted modified nucleobases are well known to those skilled in the art.

Antisense compounds may also contain one or more nucleosides having modified sugar moieties. The furanosyl sugar ring of a nucleoside can be modified in a number of ways including, but not limited to, addition of a substituent group, bridging of two non-geminal ring atoms to form a bicyclic nucleic acid (BNA) and substitution of an atom or group such as —S—, —N(R)— or —C(R₁)(R₂) for the ring oxygen at the 4′-position. Modified sugar moieties are well known and can be used to alter, typically increase, the affinity of the antisense compound for its target and/or increase nuclease resistance. A representative list of preferred modified sugars includes but is not limited to bicyclic modified sugars (BNA's), including LNA and ENA (4′-(CH₂)₂—O-2′ bridge); and substituted sugars, especially 2′-substituted sugars having a 2′-F, 2′-OCH₂ or a 2′—O(CH₂)₂—OCH₃ substituent group. Sugars can also be replaced with sugar mimetic groups among others. Methods for the preparations of modified sugars are well known to those skilled in the art.

Internucleoside linking groups link the nucleosides or otherwise modified monomer units together thereby forming an antisense compound. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, and phosphorothioates. Representative non-phosphorus containing internucleoside linking groups include, but are not limited to, methylenemethylimino (—CH.sub.2-N(CH.sub.3)—O—CH.sub.2-), thiodiester (—O—C(O)—S—), thionocarbamate (—O—C(O)(NH)—S—); siloxane (—O—Si(H).sub.2-O—); and N,N′-dimethylhydrazine (—CH.sub.2-N(CH.sub.3)—N(CH.sub.3)-). Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

As used herein the term “mimetic” refers to groups that are substituted for a sugar, a nucleobase, and/or internucleoside linkage Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein the term “nucleoside” includes, nucleosides, abasic nucleosides, modified nucleosides, and nucleosides having mimetic bases and/or sugar groups.

In the context of this disclosure, the term “oligonucleotide” refers to an oligomeric compound which is an oligomer or polymer of ribonucleic acid (RNA) or deoxyribonucleic acid (DNA). This term includes oligonucleotides composed of naturally- and non-naturally-occurring nucleobases, sugars and covalent internucleoside linkages, possibly further including non-nucleic acid conjugates.

Provided are compounds having reactive phosphorus groups useful for forming internucleoside linkages including for example phosphodiester and phosphorothioate internucleoside linkages. Methods of preparation and/or purification of precursors or antisense compounds of the instant invention are not a limitation of the compositions or methods of the invention. Methods for synthesis and purification of DNA, RNA, and the antisense compounds are well known to those skilled in the art.

As used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general a chimeric oligomeric compound will have modified nucleosides that can be in isolated positions or grouped together in regions that will define a particular motif Any combination of modifications and or mimetic groups can comprise a chimeric oligomeric compound.

Chimeric antisense compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter antisense compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Certain chimeric as well as non-chimeric antisense compounds can be further described as having a particular motif. As used herein, the term “motif” refers to the orientation of modified sugar moieties and/or sugar mimetic groups in an antisense compound relative to like or differentially modified or unmodified nucleosides. As used herein, the terms “sugars”, “sugar moieties” and “sugar mimetic groups’ are used interchangeably. Such motifs include, but are not limited to, gapped motifs, alternating motifs, fully modified motifs, hemimer motifs, blockmer motifs, and positionally modified motifs. The sequence and the structure of the nucleobases and type of internucleoside linkage is not a factor in determining the motif of an antisense compound.

As used herein, the term “gapped motif” refers to an antisense compound comprising a contiguous sequence of nucleosides that is divided into 3 regions, an internal region (gap) flanked by two external regions (wings). The regions are differentiated from each other at least by having differentially modified sugar groups that comprise the nucleosides. In some embodiments, each modified region is uniformly modified (e.g. the modified sugar groups in a given region are identical); however, other motifs can be applied to regions. For example, the wings in a gapmer could have an alternating motif. The nucleosides located in the gap of a gapped antisense compound have sugar moieties that are different than the modified sugar moieties in each of the wings.

As used herein, the term “alternating motif” refers to an antisense compound comprising a contiguous sequence of nucleosides comprising two differentially sugar modified nucleosides that alternate for essentially the entire sequence of the antisense compound, or for essentially the entire sequence of a region of an antisense compound.

As used herein, the term “fully modified motif” refers to an antisense compound comprising a contiguous sequence of nucleosides wherein essentially each nucleoside is a sugar modified nucleoside having uniform modification.

As used herein, the term “hemimer motif” refers to a sequence of nucleosides that have uniform sugar moieties (identical sugars, modified or unmodified) and wherein one of the 5′-end or the 3′-end has a sequence of from 2 to 12 nucleosides that are sugar modified nucleosides that are different from the other nucleosides in the hemimer modified antisense compound.

As used herein, the term “blockmer motif” refers to a sequence of nucleosides that have uniform sugars (identical sugars, modified or unmodified) that is internally interrupted by a block of sugar modified nucleosides that are uniformly modified and wherein the modification is different from the other nucleosides. Methods of preparation of chimeric oligonucleotide compounds are well known to those skilled in the art.

As used herein, the term “positionally modified motif” comprises all other motifs. Methods of preparation of positionally modified oligonucleotide compounds are well known to those skilled in the art.

The compounds described herein contain one or more asymmetric centers and thus give rise to enantiomers, diastereomers, and other stereoisomeric configurations that may be defined, in terms of absolute stereochemistry, as (R) or (S), .alpha. or .beta., or as (D) or (L) such as for amino acids et al. This is meant to include all such possible isomers, as well as their racemic and optically pure forms. In one aspect, antisense compounds are modified by covalent attachment of one or more conjugate groups. Conjugate groups may be attached by reversible or irreversible attachments. Conjugate groups may be attached directly to antisense compounds or by use of a linker. Linkers may be mono- or bifunctional linkers. Such attachment methods and linkers are well known to those skilled in the art. In general, conjugate groups are attached to antisense compounds to modify one or more properties. Such considerations are well known to those skilled in the art.

### Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

### Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates. The compositions and methods disclosed herein not limited by the method of oligomer purification.

### Salts, Prodrugs and Bioequivalents

The antisense compounds may comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the antisense compounds, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes, chemicals, and/or conditions. In particular, prodrug versions of the oligonucleotides of the invention are prepared as SATE ((S-acetyl-2-thioethyl) phosphate) derivatives according to the methods disclosed in WO 93/24510 or WO 94/26764. Prodrugs can also include antisense compounds wherein one or both ends comprise nucleobases that are cleaved (e.g., phosphodiester backbone linkages) to produce the smaller active compound.

The term “pharmaceutically acceptable salts” refers to physiologically and pharmaceutically acceptable salts of the antisense compounds: i.e., salts that retain the desired biological activity of the parent compound and do not impart undesired toxicological effects thereto. Sodium salts of antisense oligonucleotides are useful and are well accepted for therapeutic administration to humans. In another embodiment, sodium salts of dsRNA compounds are also provided.

### Formulations

The antisense compounds may also be admixed, encapsulated, conjugated or otherwise associated with other molecules, molecule structures or mixtures of compounds.

The antisense compounds may also include pharmaceutical compositions and formulations. The pharmaceutical compositions of the present invention may be administered in a number of ways depending upon whether local or systemic treatment is desired and upon the area to be treated.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

### Combinations

Compositions provided herein can contain two or more antisense compounds. In another related embodiment, compositions can contain one or more antisense compounds, particularly oligonucleotides, targeted to a first nucleic acid and one or more additional antisense compounds targeted to a second nucleic acid target. Alternatively, compositions can contain two or more antisense compounds targeted to different regions of the same nucleic acid target. Two or more combined compounds may be used together or sequentially. Compositions of the instant invention can also be combined with other non-antisense compound therapeutic agents.

### Nonlimiting Disclosure and Incorporation by Reference

While certain compounds, compositions and methods have been described with specificity in accordance with certain embodiments, the following examples serve only as illustrations of the compounds and methods and are not intended to limit the claims of the invention. Each of the references, GenBank accession numbers, and the like recited in the present application is incorporated herein by reference in its entirety.

### Example 1

### Cell Types and Transfection Methods

Cell Types—

The effect of oligomeric compounds on target nucleic acid expression was tested in one or more of the following cell types.

A549:

The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for use in oligomeric compound transfection experiments.

b.END:

The mouse brain endothelial cell line b.END was obtained from Dr. Werner Risau at the Max Plank Institute (Bad Nauheim, Germany) b.END cells were routinely cultured in DMEM, high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 3000 cells/well for use in oligomeric compound transfection experiments.

A10:

The rat aortic smooth muscle cell line A10 was obtained from the American Type Culture Collection (Manassas, Va.). A10 cells were routinely cultured in DMEM, high glucose (American Type Culture Collection, Manassas, Va.) supplemented with 10% fetal bovine serum (Invitrogen Life Technologies, Carlsbad, Calif.). Cells were routinely passaged by trypsinization and dilution when they reached approximately 80% confluence. Cells were seeded into 96-well plates (Falcon-Primaria #3872) at a density of approximately 2500 cells/well for use in oligomeric compound transfection experiments.

Primary Mouse Hepatocytes:

Primary mouse hepatocytes were prepared from CD-1 mice purchased from Charles River Labs. Primary mouse hepatocytes were routinely cultured in Hepatocyte Attachment Media supplemented with 10% fetal bovine serum, 1% penicillin/streptomycin, 1% antibiotic-antimitotic (Invitrogen Life Technologies, Carlsbad, Calif.) and 10 nM bovine insulin (Sigma-Aldrich, St. Louis, Mo.). Cells were seeded into 96-well plates (Falcon-Primaria #353872, BD Biosciences, Bedford, Mass.) coated with 0.1 mg/ml collagen at a density of approximately 10,000 cells/well for use in oligomeric compound transfection experiments.

Primary Rat Hepatocytes:

Primary rat hepatocytes are prepared from Sprague-Dawley rats purchased from Charles River Labs (Wilmington, Mass.) and are routinely cultured in DMEM, high glucose (Invitrogen Life Technologies, Carlsbad, Calif.) supplemented with 10% fetal bovine serum (Invitrogen Life Technologies, Carlsbad, Calif.), 100 units per mL penicillin, and 100 .micro.g/mL streptomycin (Invitrogen Life Technologies, Carlsbad, Calif.). Cells are seeded into 96-well plates (Falcon-Primaria #353872, BD Biosciences, Bedford, Mass.) at a density of 4000-6000 cells/well treatment with the oligomeric compounds of the invention.

For Northern blotting or other analysis, cells may be seeded onto 100 mm or other standard tissue culture plates and treated similarly, using appropriate volumes of medium and oligonucleotide.

Treatment with Oligomeric Compounds:

When cells reach appropriate confluency, they are treated with oligonucleotide using a transfection method as described.

Lipofectin™

When cells reached 65-75% confluency, they were treated with oligonucleotide. Oligonucleotide was mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEMT™-1 and then treated with 130 μl of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

CYTOFECTIN™:

When cells reached 65-75% confluency, they were treated with oligonucleotide. Oligonucleotide was mixed with CYTOFECTIN™ (Gene Therapy Systems, San Diego, Calif.) in OPTI-MEM-1.sup.™ reduced serum medium (Invitrogen Life Technologies, Carlsbad, Calif.) to achieve the desired concentration of oligonucleotide and a CYTOFECTIN™ concentration of 2 or 4 .micro.g/mL per 100 nM oligonucleotide. This transfection mixture was incubated at room temperature for approximately 0.5 hours. For cells grown in 96-well plates, wells were washed once with 100 .micro.L OPTI-MEM-1.sup.™ and then treated with 130 .micro.L of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

### Control Oligonucleotides

Control oligonucleotides are used to determine the optimal oligomeric compound concentration for a particular cell line. Furthermore, when oligomeric compounds of the invention are tested in oligomeric compound screening experiments or phenotypic assays, control oligonucleotides are tested in parallel with compounds of the invention.

TABLE 2
Control oligonucleotides for cell line testing, oligomeric compound screening and
phenotypic assays

| | | Species | | | SEQ |
|---|---|---|---|---|---|
| Compound | Target | of | | | ID |
| No. | Name | Target | Sequence (5′ to 3′) | Motif | NO |
| 113131 | CD86 | Human | CGTGTGTCTGTGCTAGTCCC | 5-10-5 | 19 |
| 289865 | forkhead | Human | GGCAACGTGAACAGGTCCAA | 5-10-20 | 20 |
| | box O1A | | | | |
| | (rhabdo- | | | | |
| | myosarcoma) | | | | |
| 25237 | integrin | Human | GCCCATTGCTGGACATGC | 4-10-4 | 21 |
| | beta 3 | | | | |
| 196103 | integrin | Human | AGCCCATTGCTGGACATGCA | 5-10-5 | 22 |
| | beta 3 | | | | |
| 148715 | Jagged 2 | Human; | TTGTCCCAGTCCCAGGCCTC | 5-10-5 | 23 |
| | | Mouse; | | | |
| | | Rat | | | |
| 18076 | Jun N- | Human | CTTTC^{u}CGTTGGA^{u}C^{u}CCCTGGG | 5-9-6 | 24 |
| | Terminal | | | | |
| | Kinase - 1 | | | | |
| 18078 | Jun N- | Human | GTGCG^{u}CG^{u}CGAG^{u}C^{u}C^{u}CGAAATC | 5-9-6 | 25 |
| | Terminal | | | | |
| | Kinase - 2 | | | | |
| 183881 | kinesin- | Human | ATCCAAGTGCTACTGTAGTA | 5-10-5 | 26 |
| | like 1 | | | | |
| 29848 | none | none | NNNNNNNNNNNNNNNNNNNN | 5-10-5 | 27 |
| 226844 | Notch | Human; | GCCCTCCATGCTGGCACAGG | 5-10-5 | 28 |
| | (Drosophila) | Mouse | | | |
| | homolog | | | | |
| | 1 | | | | |
| 105990 | Peroxisome | Human | AGCAAAAGATCAATCCGTTA | 5-10-5 | 29 |
| | proliferator- | | | | |
| | activated | | | | |
| | receptor | | | | |
| | gamma | | | | |
| 336806 | Raf kinase | Human | TACAGAAGGCTGGGCCTTGA | 5-10-5 | 30 |
| | C | | | | |
| 15770 | Raf kinase | Mouse; | ATGCATT^{u}CTG^{u}C^{u}C^{u}C^{u}C^{u}CAAGGA | 5-10-5 | 31 |
| | C | Murine | | | |
| | | carsoma | | | |
| | | virus; | | | |
| | | Rat | | | |

The concentration of oligonucleotide used varies from cell line to cell line. To determine the optimal oligonucleotide concentration for a particular cell line, the cells are treated with a positive control oligonucleotide at a range of concentrations. Positive controls are shown in Table 2. For human and non-human primate cells, the positive control oligonucleotide is selected from Compound No. 13650, Compound No. 336806, or Compound No. 18078. For mouse or rat cells the positive control oligonucleotide is Compound No. 15770 or Compound No. 15346. The concentration of positive control oligonucleotide that results in 80% inhibition of the target mRNA, for example, human Raf kinase C for Compound No. 13650, is then utilized as the screening concentration for new oligonucleotides in subsequent experiments for that cell line. If 80% inhibition is not achieved, the lowest concentration of positive control oligonucleotide that results in 60% inhibition of the target mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM when the antisense oligonucleotide is transfected using a liposome reagent and 1 .micro.M to 40 .micro.M when the antisense oligonucleotide is transfected by electroporation.

### Example 2

### Real-Time Quantitative PCR Analysis of LMW-PTPase mRNA Levels

Quantitation of LMW-PTPase mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the LMW-PTPase being measured were evaluated for their ability to be “multiplexed” with a GAPDH amplification reaction. After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 .micro.L PCR cocktail (2.5×PCR buffer minus MgCl.sub.2, 6.6 mM MgCl.sub.2, 375 .micro.M each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 .micro.L total RNA solution (20-200 ng). The RT reaction was carried out by incubation for 30 minutes at 48.deg.C. Following a 10 minute incubation at 95.deg.C. to activate the PLATINUM® Taq, 40 cycles of a two-step PCR protocol were carried out: 95.deg.C. for 15 seconds (denaturation) followed by 60.deg.C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc. Eugene, Oreg.).

170 .micro.L of RiboGreen™ working reagent (RiboGreen™ reagent diluted 1:350 in 10 mM Tris-HCl, 1 mM EDTA, pH 7.5) was pipetted into a 96-well plate containing 30 .micro.L purified cellular RNA. The plate was read in a CytoFluor 4000 (PE Applied Biosystems) with excitation at 485 nm and emission at 530 nm.

The GAPDH PCR probes have JOE covalently linked to the 5′ end and TAMRA or MGB covalently linked to the 3′ end, where JOE is the fluorescent reporter dye and TAMRA or MGB is the quencher dye. In some cell types, primers and probe designed to a GAPDH sequence from a different species are used to measure GAPDH expression. For example, a human GAPDH primer and probe set is used to measure GAPDH expression in monkey-derived cells and cell lines.

Probes and primers for use in real-time PCR were designed to hybridize to target-specific sequences. The primers and probes and the target nucleic acid sequences to which they hybridize are presented in Table 3. The target-specific PCR probes have FAM covalently linked to the 5′ end and TAMRA or MGB covalently linked to the 3′ end, where FAM is the fluorescent dye and TAMRA or MGB is the quencher dye.

TABLE 3
LMW-PTPase-specific primers and probes for use in real-time PCR

| Target | | Sequence | | SEQ ID |
|---|---|---|---|---|
| Name | Species | Description | Sequence (5′ to 3′) | NO |
| GAPDH | Human | Forward Primer | CAACGGATTTGGTCGTATTGG | 32 |
| GAPDH | Human | Reverse Primer | GGCAACAATATCCACTTTACCAGAGT | 33 |
| GAPDH | Human | Probe | CGCCTGGTCACCAGGGCTGCT | 34 |
| GAPDH | Human | Forward Primer | GAAGGTGAAGGTCGGAGTC | 35 |
| GAPDH | Human | Reverse Primer | GAAGATGGTGATGGGATTTC | 36 |
| GAPDH | Human | Probe | CAAGCTTCCCGTTCTCAGCC | 37 |
| GAPDH | Human | Forward Primer | GAAGGTGAAGGTCGGAGTC | 35 |
| GAPDH | Human | Reverse Primer | GAAGATGGTGATGGGATTTC | 36 |
| GAPDH | Human | Probe | TGGAATCATATTGGAACATG | 38 |
| GAPDH | Mouse | Forward Primer | GGCAAATTCAACGGCACAGT | 39 |
| GAPDH | Mouse | Reverse Primer | GGGTCTCGCTCCTGGAAGAT | 40 |
| GAPDH | Mouse | Probe | AAGGCCGAGAATGGGAAGCTTGTCATC | 41 |
| GAPDH | Rat | Forward Primer | TGTTCTAGAGACAGCCGCATCTT | 42 |
| GAPDH | Rat | Reverse Primer | CACCGACCTTCACCATCTTGT | 43 |
| GAPDH | Rat | Probe | TTGTGCAGTGCCAGCCTCGTCTCA | 44 |

### Example 3

### Antisense Inhibition of Human LMW-PTPase Expression by Oligomeric Compounds

A series of antisense compounds was designed to target different regions of human LMW-PTPase RNA, using published sequences or portions of published sequences as cited in Table 1. The designed antisense compounds are complementary to one or more of the target nucleic acids in Table 1. The start and stop sites on the target nucleic acids for each antisense compound are presented in Tables 4a, b, c and d.

TABLE 4b
SEQ ID NO: 4

| Compound # | Start Site | Stop Site |
|---|---|---|
| 356739 | 65 | 84 |
| 356740 | 73 | 92 |
| 356741 | 78 | 97 |
| 356742 | 87 | 106 |
| 356743 | 103 | 122 |
| 356744 | 117 | 136 |
| 288247 | 127 | 146 |
| 356745 | 132 | 151 |
| 356746 | 148 | 167 |
| 356747 | 170 | 189 |
| 356800 | 177 | 196 |
| 356750 | 201 | 220 |
| 356751 | 242 | 261 |
| 356752 | 253 | 272 |
| 356753 | 272 | 291 |
| 356754 | 277 | 296 |
| 356755 | 312 | 331 |
| 288270 | 328 | 347 |
| 288271 | 333 | 352 |
| 288273 | 338 | 357 |
| 288274 | 340 | 359 |
| 288275 | 343 | 362 |
| 288276 | 345 | 364 |
| 356756 | 353 | 372 |
| 356757 | 381 | 400 |
| 356758 | 415 | 434 |
| 356759 | 441 | 460 |
| 356760 | 451 | 470 |
| 356761 | 459 | 478 |
| 356762 | 464 | 483 |
| 356763 | 473 | 492 |
| 356764 | 489 | 508 |
| 356765 | 524 | 543 |
| 356766 | 536 | 555 |
| 356767 | 547 | 566 |
| 356768 | 567 | 586 |
| 356769 | 591 | 610 |
| 356770 | 601 | 620 |
| 356771 | 619 | 638 |
| 356772 | 637 | 656 |
| 356773 | 668 | 687 |
| 356774 | 727 | 746 |
| 356775 | 746 | 765 |
| 356776 | 751 | 770 |
| 356777 | 757 | 776 |
| 356778 | 840 | 859 |
| 356779 | 860 | 879 |
| 356780 | 873 | 892 |
| 356781 | 888 | 907 |
| 356782 | 905 | 924 |
| 356783 | 961 | 980 |
| 356784 | 1022 | 1041 |
| 356785 | 1030 | 1049 |
| 356786 | 1050 | 1069 |
| 356787 | 1058 | 1077 |
| 356788 | 1093 | 1112 |
| 356789 | 1111 | 1130 |
| 356790 | 1118 | 1137 |
| 356791 | 1125 | 1144 |
| 356792 | 1170 | 1189 |
| 356793 | 1184 | 1203 |
| 356794 | 1227 | 1246 |
| 356795 | 1259 | 1278 |
| 356796 | 1288 | 1307 |
| 356797 | 1387 | 1406 |
| 356798 | 1414 | 1433 |
| 356799 | 1478 | 1497 |

TABLE 4a
SEQ ID NO: 3

| Compound # | Start Site | Stop Site |
|---|---|---|
| 356739 | 65 | 84 |
| 356740 | 73 | 92 |
| 356741 | 78 | 97 |
| 356742 | 87 | 106 |
| 356743 | 103 | 122 |
| 356744 | 117 | 136 |
| 288247 | 127 | 146 |
| 356745 | 132 | 151 |
| 356746 | 148 | 167 |
| 356747 | 170 | 189 |
| 356748 | 190 | 209 |
| 356801 | 291 | 310 |
| 356755 | 312 | 331 |
| 288270 | 328 | 347 |
| 288271 | 333 | 352 |
| 288273 | 338 | 357 |
| 288274 | 340 | 359 |
| 288275 | 343 | 362 |
| 288276 | 345 | 364 |
| 356756 | 353 | 372 |
| 356757 | 381 | 400 |
| 356758 | 415 | 434 |
| 356759 | 441 | 460 |
| 356760 | 451 | 470 |
| 356761 | 459 | 478 |
| 356762 | 464 | 483 |
| 356763 | 473 | 492 |
| 356764 | 489 | 508 |
| 356765 | 524 | 543 |
| 356766 | 536 | 555 |
| 356767 | 547 | 566 |
| 356768 | 567 | 586 |
| 356769 | 591 | 610 |
| 356770 | 601 | 620 |
| 356771 | 619 | 638 |
| 356772 | 637 | 656 |
| 356773 | 668 | 687 |
| 356774 | 727 | 746 |
| 356775 | 746 | 765 |
| 356776 | 751 | 770 |
| 356777 | 757 | 776 |
| 356778 | 840 | 859 |
| 356779 | 860 | 879 |
| 356780 | 873 | 892 |
| 356781 | 888 | 907 |
| 356782 | 905 | 924 |
| 356783 | 961 | 980 |
| 356784 | 1022 | 1041 |
| 356785 | 1030 | 1049 |
| 356786 | 1050 | 1069 |
| 356787 | 1058 | 1077 |
| 356788 | 1093 | 1112 |
| 356789 | 1111 | 1130 |
| 356790 | 1118 | 1137 |
| 356791 | 1125 | 1144 |
| 356792 | 1170 | 1189 |
| 356793 | 1184 | 1203 |
| 356794 | 1227 | 1246 |
| 356795 | 1259 | 1278 |
| 356796 | 1288 | 1307 |
| 356797 | 1387 | 1406 |
| 356798 | 1414 | 1433 |
| 356799 | 1478 | 1497 |

TABLE 4c
SEQ ID NO: 5

| Compound # | Start Site | Stop Site |
|---|---|---|
| 356739 | 65 | 84 |
| 356740 | 73 | 92 |
| 356741 | 78 | 97 |
| 356742 | 87 | 106 |
| 356743 | 103 | 122 |
| 356744 | 117 | 136 |
| 288247 | 127 | 146 |
| 356745 | 132 | 151 |
| 356746 | 148 | 167 |
| 356747 | 170 | 189 |
| 356748 | 190 | 209 |
| 356749 | 204 | 223 |
| 356750 | 230 | 249 |
| 356751 | 271 | 290 |
| 356752 | 282 | 301 |
| 356753 | 301 | 320 |
| 356754 | 306 | 325 |
| 356755 | 341 | 360 |
| 288270 | 357 | 376 |
| 288271 | 362 | 381 |
| 288273 | 367 | 386 |
| 288274 | 369 | 388 |
| 288275 | 372 | 391 |
| 288276 | 374 | 393 |
| 356756 | 382 | 401 |
| 356757 | 410 | 429 |
| 356758 | 444 | 463 |
| 356759 | 470 | 489 |
| 356760 | 480 | 499 |
| 356761 | 488 | 507 |
| 356762 | 493 | 512 |
| 356763 | 502 | 521 |
| 356764 | 518 | 537 |
| 356765 | 553 | 572 |
| 356766 | 565 | 584 |
| 356767 | 576 | 595 |
| 356768 | 596 | 615 |
| 356769 | 620 | 639 |
| 356770 | 630 | 649 |
| 356771 | 648 | 667 |
| 356772 | 666 | 685 |
| 356773 | 697 | 716 |
| 356774 | 756 | 775 |
| 356775 | 775 | 794 |
| 356776 | 780 | 799 |
| 356777 | 786 | 805 |
| 356778 | 869 | 888 |
| 356779 | 889 | 908 |
| 356780 | 902 | 921 |
| 356781 | 917 | 936 |
| 356782 | 934 | 953 |
| 356783 | 990 | 1009 |
| 356784 | 1051 | 1070 |
| 356785 | 1059 | 1078 |
| 356786 | 1079 | 1098 |
| 356787 | 1087 | 1106 |
| 356788 | 1122 | 1141 |
| 356789 | 1140 | 1159 |
| 356790 | 1147 | 1166 |
| 356791 | 1154 | 1173 |
| 356792 | 1199 | 1218 |
| 356793 | 1213 | 1232 |
| 356794 | 1256 | 1275 |
| 356795 | 1288 | 1307 |
| 356796 | 1317 | 1336 |
| 356797 | 1416 | 1435 |
| 356798 | 1443 | 1462 |
| 356799 | 1507 | 1526 |

TABLE 4d
SEQ ID NO: 6

| Compound # | Start Site | Stop Site |
|---|---|---|
| 356739 | 465 | 484 |
| 356740 | 473 | 492 |
| 356741 | 478 | 497 |
| 356742 | 487 | 506 |
| 356731 | 1499 | 1518 |
| 356732 | 2753 | 2772 |
| 356733 | 7057 | 7076 |
| 356744 | 7375 | 7394 |
| 288247 | 7385 | 7404 |
| 356745 | 7390 | 7409 |
| 356746 | 7406 | 7425 |
| 356734 | 7435 | 7454 |
| 356748 | 7545 | 7564 |
| 356750 | 7711 | 7730 |
| 356751 | 7752 | 7771 |
| 356752 | 7763 | 7782 |
| 356753 | 7782 | 7801 |
| 356754 | 7787 | 7806 |
| 356735 | 10635 | 10654 |
| 356755 | 10656 | 10675 |
| 288270 | 10672 | 10691 |
| 288271 | 10677 | 10696 |
| 288273 | 10682 | 10701 |
| 288274 | 10684 | 10703 |
| 288275 | 10687 | 10706 |
| 356736 | 10697 | 10716 |
| 356737 | 10721 | 10740 |
| 356738 | 12475 | 12494 |
| 356757 | 12503 | 12522 |
| 356758 | 12537 | 12556 |
| 356759 | 12563 | 12582 |
| 356763 | 12736 | 12755 |
| 356764 | 12752 | 12771 |
| 356765 | 12787 | 12806 |
| 356766 | 12799 | 12818 |
| 356767 | 12810 | 12829 |
| 356768 | 12830 | 12849 |
| 356769 | 12854 | 12873 |
| 356770 | 12864 | 12883 |
| 356771 | 12882 | 12901 |
| 356772 | 12900 | 12919 |
| 356773 | 12931 | 12950 |
| 356774 | 12990 | 13009 |
| 356775 | 13009 | 13028 |
| 356776 | 13014 | 13033 |
| 356777 | 13020 | 13039 |
| 356778 | 13103 | 13122 |
| 356779 | 13123 | 13142 |
| 356780 | 13136 | 13155 |
| 356781 | 13151 | 13170 |
| 356782 | 13168 | 13187 |
| 356783 | 13224 | 13243 |
| 356784 | 13285 | 13304 |
| 356785 | 13293 | 13312 |
| 356786 | 13313 | 13332 |
| 356787 | 13321 | 13340 |
| 356788 | 13356 | 13375 |
| 356789 | 13374 | 13393 |
| 356790 | 13381 | 13400 |
| 356791 | 13388 | 13407 |
| 356792 | 13433 | 13452 |
| 356793 | 13447 | 13466 |
| 356794 | 13490 | 13509 |
| 356795 | 13522 | 13541 |
| 356796 | 13551 | 13570 |
| 356797 | 13650 | 13669 |
| 356798 | 13677 | 13696 |
| 356799 | 13741 | 13760 |

As stated above, antisense oligonucleotides directed to a target or more preferably to an active target segment can be from about 13 to about 80 linked nucleobases. The following Table 4e provides a non-limiting example of such antisense oligonucleotides targeting SEQ ID NO 1.

TABLE 4e
Antisense Oligonucleotides from about 13 to about 35 Nucleobases

| Sequence | Length |
|---|---|
| CCATGATTTCTTAGGCAGCT | 20 nucleobases (SEQ ID NO: 76) |
| AATGCCATGATTTCT | 15 nucleobases (SEQ ID NO: 356) |
| GCCATGATTTCTTAG | 15 nucleobases (SEQ ID NO: 357) |
| ATGCCATGATTTC | 13 nucleobases (SEQ ID NO: 358) |
| AATGCCATGATTTCTTAGGCAGCTC | 24 nucleobases (SEQ ID NO: 359) |
| TTTCTTAGGCAGCT | 14 nucleobases (SEQ ID NO: 360) |
| TGTGAATGCCATGATTTCTTAGGCAGCTCACAGCT | 35 nucleobases (SEQ ID NO: 361) |
| CCATGATTTCTTAGGCAGCTCACAGCT | 27 nucleobases (SEQ ID NO: 362) |
| GATTTCTTAGGCAGCTCACAGCT | 22 nucleobases (SEQ ID NO: 363) |

Antisense oligonucleotides directed to a target or more preferably to an active target segment can also contain mismatched nucleobases when compared to the target sequence. The following Table 4f provides a non-limiting example of such antisense oligonucleotides targeting nucleobases 282 to 301 of SEQ ID NO 5. Mismatched nucleobases are underlined. One ordinarily skilled in the art understands that antisense compounds can tolerate mismatches yet still retain their ability to hybridize with a target site and modulate the target nucleic acid through antisense mechanisms.

TABLE 4f
Antisense Oligonucleotides from about 1-3 Nucleobases Mismatched to the Target Sequence

| | Number of mismatches to |
|---|---|
| Sequence | SEQ ID NO: 5 |
| CCATGATTTCTTAGGCAGCT (SEQ ID NO: 76) | None |
| CCATGATTTCTTAGGCATCT (SEQ ID NO: 364) | One mismatch |
| CCATGATCTCTTAGGCAGCT (SEQ ID NO: 365) | One mismatch |
| CCATGATTTCTTAGGCACAT (SEQ ID NO: 366) | Two mismatches |
| GCATGATTTCTTAGGCCGCT (SEQ ID NO: 367) | Two mismatches |
| CCTTGATACCTTAGGCAGCT (SEQ ID NO: 368) | Three mismatches |

Antisense compounds were designed against one or more of the human LMW-PTPase target nucleic acids sequences published in table 1 and were screened in vitro to determine the compound's ability to modulate expression of a target nucleic acid that encodes LMW-PTPase. The compounds shown in Table 5 are all chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the primer-probe set designed to hybridize to human LMW-PTPase (Table 2). Data are averages from two experiments in which A549 cells were treated with 65 nM of the disclosed oligomeric compounds using LIPOFECTIN™. A reduction in expression is expressed as percent inhibition in Table 5. If present, “N.D.” indicates “not determined”. The control oligomeric compound used was SEQ ID NO: 25.

TABLE 5
Inhibition of human LMW-PTPase mRNA levels by chimeric oligonucleotides having 2′-
MOE wings and deoxy gap

| | Target | | | | |
|---|---|---|---|---|---|
| Compound | SEQ ID | Target | | | SEQ ID |
| No. | NO | Site | Sequence (5′ to 3′) | % Inhib | NO |
| 356801 | 3 | 291 | CTTTGGTAATCTGCCGGGCA | 36 | 54 |
| 288247 | 4 | 127 | ACTGCTTCTGCAATGGGTGA | 67 | 55 |
| 356800 | 4 | 177 | CAATGACCCAATTCTCTGAG | 16 | 56 |
| 288270 | 4 | 328 | TCCATACATAGTATATAATC | 39 | 57 |
| 288271 | 4 | 333 | TTTCATCCATACATAGTATA | 29 | 58 |
| 288273 | 4 | 338 | ATTGCTTTCATCCATACATA | 54 | 59 |
| 288274 | 4 | 340 | AGATTGCTTTCATCCATACA | 51 | 60 |
| 288275 | 4 | 343 | CTCAGATTGCTTTCATCCAT | 63 | 61 |
| 288276 | 4 | 345 | CTCTCAGATTGCTTTCATCC | 56 | 62 |
| 356739 | 5 | 65 | AGCCTGTTCCGCCATCTTCC | 74 | 63 |
| 356740 | 5 | 73 | GACTTGGTAGCCTGTTCCGC | 67 | 64 |
| 356741 | 5 | 78 | GCACGGACTTGGTAGCCTGT | 68 | 65 |
| 356742 | 5 | 87 | ACACAAACAGCACGGACTTG | 35 | 66 |
| 356743 | 5 | 103 | CAAATGTTACCCAGACACAC | 33 | 67 |
| 356744 | 5 | 117 | CAATGGGTGATCGACAAATG | 55 | 68 |
| 356745 | 5 | 132 | TGAAAACTGCTTCTGCAATG | 54 | 69 |
| 356746 | 5 | 148 | TCGGTTACAAGTTTCCTGAA | 64 | 70 |
| 356747 | 5 | 170 | CCAATTCTCTGAGATGTTTT | 68 | 71 |
| 356748 | 5 | 190 | GTTGCCGCGCTGTCTACCCT | 70 | 72 |
| 356749 | 5 | 204 | ATGACCCACCGGAAGTTGCC | 22 | 73 |
| 356750 | 5 | 230 | TCCAGTCAGAAACAGCACCG | 50 | 74 |
| 356751 | 5 | 271 | TAGGCAGCTCACAGCTCTTG | 59 | 75 |
| 356752 | 5 | 282 | CCATGATTTCTTAGGCAGCT | 76 | 76 |
| 356753 | 5 | 301 | TTTATGGGCTGTGTGAATGC | 56 | 77 |
| 356754 | 5 | 306 | CTTGCTTTATGGGCTGTGTG | 70 | 78 |
| 356755 | 5 | 341 | AATCAAATGTGGCAAAATCT | 51 | 79 |
| 356756 | 5 | 382 | ATTCAAATCTCTCAGATTGC | 52 | 80 |
| 356757 | 5 | 410 | TGCAGGTTTTAACTTGATTA | 57 | 81 |
| 356758 | 5 | 444 | GGATCATAGCTCCCAAGTAG | 57 | 82 |
| 356759 | 5 | 470 | GATCTTCAATAATAAGTTGT | 55 | 83 |
| 356760 | 5 | 480 | CCATAATAGGGATCTTCAAT | 26 | 84 |
| 356761 | 5 | 488 | AGTCATTCCCATAATAGGGA | 52 | 85 |
| 356762 | 5 | 493 | GTCAGAGTCATTCCCATAAT | 60 | 86 |
| 356763 | 5 | 502 | CGTCTCAAAGTCAGAGTCAT | 62 | 87 |
| 356764 | 5 | 518 | CACACTGCTGGTACACCGTC | 74 | 88 |
| 356765 | 5 | 553 | GTGGGCCTTCTCCAAGAACG | 43 | 89 |
| 356766 | 5 | 565 | GAACCTGCCTCAGTGGGCCT | 74 | 90 |
| 356767 | 5 | 576 | CAGCAGGGCACGAACCTGCC | 38 | 91 |
| 356768 | 5 | 596 | GGGTCTAGTCAGGCTGGCCG | 67 | 92 |
| 356769 | 5 | 620 | TGAGAAATGCAGGACCTCAG | 80 | 93 |
| 356770 | 5 | 630 | ACACACCGACTGAGAAATGC | 64 | 94 |
| 356771 | 5 | 648 | GGGCCCTGGAACGTGATTAC | 55 | 95 |
| 356772 | 5 | 666 | AACAAAGAGCTGGGCTTTGG | 71 | 96 |
| 356773 | 5 | 697 | CTTTTTAAGGTAAGAAACAG | 25 | 97 |
| 356774 | 5 | 756 | TGAATCAAAGATTTTTATTG | 15 | 98 |
| 356775 | 5 | 775 | AAATACCCCATAAGCTGTCT | 45 | 99 |
| 356776 | 5 | 780 | GCTTAAAATACCCCATAAGC | 61 | 100 |
| 356777 | 5 | 786 | AAGAATGCTTAAAATACCCC | 27 | 101 |
| 356778 | 5 | 869 | CAAGTGAGGTTTTCCTTCAT | 67 | 102 |
| 356779 | 5 | 889 | TAGATGTTGACCTGGGCCTT | 61 | 103 |
| 356780 | 5 | 902 | GTCTCAACAGGCTTAGATGT | 53 | 104 |
| 356781 | 5 | 917 | GACTCGATTATCTAAGTCTC | 57 | 105 |
| 356782 | 5 | 934 | AACCTACTGAAGAGGTAGAC | 76 | 106 |
| 356783 | 5 | 990 | AAGAGAGAGGTAGCACTGGG | 45 | 107 |
| 356784 | 5 | 1051 | CTAATCTAGACTGTGAGCTC | 79 | 108 |
| 356785 | 5 | 1059 | AAACACTTCTAATCTAGACT | 21 | 109 |
| 356786 | 5 | 1079 | CTATGGGTGTGTAGAAATTA | 67 | 110 |
| 356787 | 5 | 1087 | AGTGTGCACTATGGGTGTGT | 51 | 111 |
| 356788 | 5 | 1122 | AAATGTTTCTCTCTTCCCTA | 31 | 112 |
| 356789 | 5 | 1140 | GCCAACGACTGATTCCATAA | 87 | 113 |
| 356790 | 5 | 1147 | TGAAGGTGCCAACGACTGAT | 79 | 114 |
| 356791 | 5 | 1154 | GAAGTATTGAAGGTGCCAAC | 80 | 115 |
| 356792 | 5 | 1199 | GCCAATGGGCTGACCTCCTC | 77 | 116 |
| 356793 | 5 | 1213 | TGGTTCAGATGGGAGCCAAT | 66 | 117 |
| 356794 | 5 | 1256 | AAGTGTCCTTCTTTCTGGAT | 67 | 118 |
| 356795 | 5 | 1288 | CATATTCCTCAACTGACCAT | 31 | 119 |
| 356796 | 5 | 1317 | TTTGGGTTACATGTGCATAT | 73 | 120 |
| 356797 | 5 | 1416 | TGATGAAGAATACTTATTCA | 49 | 121 |
| 356798 | 5 | 1443 | ACATCTGCCTATACATTTAT | 24 | 122 |
| 356799 | 5 | 1507 | TCCCCAGTTTATTTTGAAAT | 37 | 123 |
| 356731 | 6 | 1499 | GGAAGCAACTCATGATCTGG | 63 | 124 |
| 356732 | 6 | 2753 | AATGCATGCCATATAGTAGA | 43 | 125 |
| 356733 | 6 | 7057 | CTAATGATCCAGGAGTGAAT | 39 | 126 |
| 356734 | 6 | 7435 | TGGTACTTACATTCTCTGAG | 23 | 127 |
| 356735 | 6 | 10635 | CTTTGGTAATCTAAAATTGA | 15 | 128 |
| 356736 | 6 | 10697 | ACAGGATTACCTCAGATTGC | 53 | 129 |
| 356737 | 6 | 10721 | GTTGAACAGAAATATTCTTC | 13 | 130 |
| 356738 | 6 | 12475 | ATTCAAATCTCTGTAAAATT | 14 | 131 |

The screen identified active target segments within the human LMW-PTPase mRNA sequence, specifically SEQ ID NOS: 3, 4 and 5. Each active target segment was targeted by at least one active antisense oligonucleotide. These active target regions identified for SEQ ID NO: 3 include nucleotides 1111 to 1189 (Region A) with an average inhibition of 80.5%, nucleotides 489 to 656 (Region B) with an average inhibition of 62.8%, nucleotides 536 to 656 (Region C) with an average inhibition of 64.1%, nucleotides 489 to 610 (Region D) with an average inhibition of 62.6%, nucleotides 1111 to 1203 (Region E) with an average inhibition of 77.6%, nucleotides 840 to 924 (Region F) with an average inhibition of 62.8%, nucleotides 1022 to 1069 (Region G) with an average inhibition of 55.6%, nucleotides 65 to 209 (Region H) with an average inhibition of 59.5%, nucleotides 65 to 136 (Region I) with an average inhibition of 55.2%, nucleotides 117 to 209 (Region J) with an average inhibition of 63.0% and nucleotides 338 to 460 (Region K) with an average inhibition of 55.6%. Over half of the oligonucleotides tested in this region inhibited expression by greater than 63%. Identification of these regions allows for the design of antisense oligonucleotides that modulate the expression of LMW-PTPase.

The active target regions identified for SEQ ID NO: 4 include nucleotides nucleotides 65 to 189 (Region AA) with an average inhibition of 58.4%, nucleotides 65 to 146 (Region AB) with an average inhibition of 56.8%, nucleotides 127 to 189 (Region AC) with an average inhibition of 63.2%, nucleotides 338 to 460 (Region AD) with an average inhibition of 55.6%, nucleotides 489 to 610 (Region AE) with an average inhibition of 62.6%, nucleotides 536 to 656 (Region AF) with an average inhibition of 64.1%, nucleotides 489 to 656 (Region AG) with an average inhibition of 62.8%, nucleotides 840 to 924 (Region AH) with an average inhibition of 62.8%, nucleotides 1022 to 1069 (Region AI) with an average inhibition of 55.6%, nucleotides 1111 to 1189 (Region AJ) with an average inhibition of 80.5% and nucleotides 1111 to 1203 (Region AK) with an average inhibition of 77.6%.

Active target regions have also been identified for SEQ ID NO: 5. These active target regions include nucleotides 65 to 136 (Region BA) with an average inhibition of 55.2%, nucleotides 117 to 209 (Region BB) with an average inhibition of 63.0%, nucleotides 65 to 209 (Region BC) with an average inhibition of 59.5%, nucleotides 367 to 489 (Region BD) with an average inhibition of 55.6%, nucleotides 518 to 639 (Region BE) with an average inhibition of 62.6%, nucleotides 565 to 685 (Region BF) with an average inhibition of 64.1%, nucleotides 518 to 685 (Region BG) with an average inhibition of 62.8%, nucleotides 689 to 953 (Region BH) with an average inhibition of 62.8%, nucleotides 1051 to 1098 (Region BI) with an average inhibition of 55.6%, nucleotides 1140 to 1218 (Region BJ) with an average inhibition of 80.53% and nucleotides 1140 to 1232 (Region BK) with an average inhibition of 77.6%.

### Example 4

### Antisense Inhibition of Mouse LMW-PTPase Expression by Oligomeric Compounds

Antisense compounds were designed against one or more of the mouse LMW-PTPase target nucleic acid sequences cited in Table 1 and were screened in vitro to determine the compound's ability to modulate expression of a target nucleic acid that encodes LMW-PTPase. The compounds shown in Table 6 are all chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the primer-probe set designed to hybridize to mouse LMW-PTPase (Table 2). Data are averages from two experiments in which b.END cells were treated with 75 nM of the disclosed oligomeric compounds using LIPOFECTIN™. A reduction in expression is expressed as percent inhibition in Table 6. The control oligomeric compound used was SEQ ID NO: 25. If present, “N.D.” indicates “not determined”.

TABLE 6
Inhibition of mouse LMW-PTPase mRNA levels by chimeric oligonucleotides having 2′-MOE
wings and deoxy gap

| | Target | | | | |
|---|---|---|---|---|---|
| Compound | SEQ ID | Target | | | SEQ ID |
| No. | NO | Site | Sequence (5′ to 3′) | % Inhib | NO |
| 288290 | 11 | 207 | CTGTCTGACTCAAATGCTTT | 69 | 132 |
| 288291 | 11 | 252 | GGTCAGAGGTTTAGTTAGTC | 80 | 133 |
| 288292 | 11 | 493 | TCCGTCTGCGGTTTTATGTA | 4 | 134 |
| 288293 | 11 | 982 | GTGGTGCTCTGTTGAGGTGT | 0 | 135 |
| 288216 | 12 | 3 | TTGCTTAGTCTATAACTGAC | 0 | 136 |
| 288217 | 12 | 13 | ATGATGGAGATTGCTTAGTC | 0 | 137 |
| 288218 | 12 | 24 | AAATATGCTAAATGATGGAG | 0 | 138 |
| 288219 | 12 | 40 | GCTTCCTGTGCACCAGAAAT | 64 | 139 |
| 288220 | 12 | 48 | CTCACGTTGCTTCCTGTGCA | 0 | 140 |
| 288221 | 12 | 79 | ACTTTGTAATGGGAGTAGAT | 3 | 141 |
| 288222 | 12 | 90 | TATAATGGTAGACTTTGTAA | 0 | 142 |
| 288223 | 12 | 114 | TAGAGAATGCAAGCATATCA | 0 | 143 |
| 288224 | 12 | 123 | TTCAATTAATAGAGAATGCA | 0 | 144 |
| 288225 | 12 | 149 | ACATATACACATGAGTTGTA | 0 | 145 |
| 288226 | 12 | 159 | CTTTGTAATGACATATACAC | 0 | 146 |
| 288227 | 12 | 164 | AAACTCTTTGTAATGACATA | 0 | 147 |
| 288228 | 12 | 179 | TGCTTCCATGAAGCAAAACT | 0 | 148 |
| 288229 | 12 | 189 | GATACTTTCATGCTTCCATG | 0 | 149 |
| 288230 | 12 | 196 | AATATGTGATACTTTCATGC | 0 | 150 |
| 288231 | 12 | 202 | GCCATAAATATGTGATACTT | 0 | 151 |
| 288232 | 12 | 232 | AGACCCTCAATTTCTCTAAT | 14 | 152 |
| 288233 | 12 | 248 | TGCCATGTTTCGGTGCAGAC | 75 | 153 |
| 288234 | 12 | 253 | ACCTCTGCCATGTTTCGGTG | 64 | 154 |
| 288235 | 12 | 266 | TGACTTGGACCCAACCTCTG | 59 | 155 |
| 288236 | 12 | 273 | ACAGCACTGACTTGGACCCA | 75 | 156 |
| 288237 | 12 | 277 | ACGAACAGCACTGACTTGGA | 61 | 157 |
| 288238 | 12 | 281 | ACACACGAACAGCACTGACT | 62 | 158 |
| 288239 | 12 | 289 | TTACCGAGACACACGAACAG | 52 | 159 |
| 288240 | 12 | 293 | AATGTTACCGAGACACACGA | 53 | 160 |
| 288241 | 12 | 296 | GCAAATGTTACCGAGACACA | 56 | 161 |
| 288242 | 12 | 304 | GGTGACCGGCAAATGTTACC | 68 | 162 |
| 288243 | 12 | 306 | TGGGTGACCGGCAAATGTTA | 61 | 163 |
| 288244 | 12 | 309 | CAATGGGTGACCGGCAAATG | 62 | 164 |
| 288245 | 12 | 310 | GCAATGGGTGACCGGCAAAT | 81 | 165 |
| 288246 | 12 | 317 | TGCTTCTGCAATGGGTGACC | 77 | 166 |
| 288247 | 12 | 319 | ACTGCTTCTGCAATGGGTGA | 83 | 55 |
| 288248 | 12 | 321 | ATACTGCTTCTGCAATGGGT | 73 | 167 |
| 288249 | 12 | 323 | GAATACTGCTTCTGCAATGG | 76 | 168 |
| 288250 | 12 | 325 | CTGAATACTGCTTCTGCAAT | 59 | 169 |
| 288251 | 12 | 328 | TTCCTGAATACTGCTTCTGC | 73 | 170 |
| 288252 | 12 | 334 | ACCAGTTTCCTGAATACTGC | 79 | 171 |
| 288253 | 12 | 338 | AGTTACCAGTTTCCTGAATA | 63 | 172 |
| 288254 | 12 | 343 | TCATCAGTTACCAGTTTCCT | 57 | 173 |
| 288255 | 12 | 347 | CTTTTCATCAGTTACCAGTT | 63 | 174 |
| 288256 | 12 | 350 | AACCTTTTCATCAGTTACCA | 67 | 175 |
| 288257 | 12 | 358 | TTATCTGAAACCTTTTCATC | 48 | 176 |
| 288258 | 12 | 360 | AATTATCTGAAACCTTTTCA | 49 | 177 |
| 288259 | 12 | 365 | GGCCCAATTATCTGAAACCT | 57 | 178 |
| 288260 | 12 | 367 | ATGGCCCAATTATCTGAAAC | 43 | 179 |
| 288261 | 12 | 375 | TGCTGTCAATGGCCCAATTA | 62 | 180 |
| 288262 | 12 | 379 | GCGCTGCTGTCAATGGCCCA | 61 | 181 |
| 288263 | 12 | 406 | GGCCGGCCCACGTTCCAGTC | 65 | 182 |
| 288264 | 12 | 493 | GCAAAGTCTTCTTTTGTAAT | 57 | 183 |
| 288265 | 12 | 499 | AATGTGGCAAAGTCTTCTTT | 54 | 184 |
| 288266 | 12 | 505 | TAATCGAATGTGGCAAAGTC | 59 | 185 |
| 288267 | 12 | 510 | GTATATAATCGAATGTGGCA | 80 | 186 |
| 288268 | 12 | 511 | AGTATATAATCGAATGTGGC | 76 | 187 |
| 288269 | 12 | 518 | CATACATAGTATATAATCGA | 52 | 188 |
| 288270 | 12 | 520 | TCCATACATAGTATATAATC | 60 | 57 |
| 288271 | 12 | 525 | TTTCATCCATACATAGTATA | 57 | 58 |
| 288272 | 12 | 526 | CTTTCATCCATACATAGTAT | 57 | 189 |
| 288273 | 12 | 530 | ATTGCTTTCATCCATACATA | 71 | 59 |
| 288274 | 12 | 532 | AGATTGCTTTCATCCATACA | 70 | 60 |
| 288275 | 12 | 535 | CTCAGATTGCTTTCATCCAT | 73 | 61 |
| 288276 | 12 | 537 | CTCTCAGATTGCTTTCATCC | 78 | 62 |
| 288277 | 12 | 538 | TCTCTCAGATTGCTTTCATC | 66 | 190 |
| 288278 | 12 | 540 | GATCTCTCAGATTGCTTTCA | 70 | 191 |
| 288279 | 12 | 545 | ATTGAGATCTCTCAGATTGC | 61 | 192 |
| 288280 | 12 | 549 | TTCTATTGAGATCTCTCAGA | 65 | 193 |
| 288281 | 12 | 572 | GCAGTTTTTAACTTGATTAC | 61 | 194 |
| 288282 | 12 | 626 | AATGATGAGCTGTTTCTGTG | 53 | 195 |
| 288283 | 12 | 636 | AGGGATCTTCAATGATGAGC | 59 | 196 |
| 288284 | 12 | 639 | AATAGGGATCTTCAATGATG | 48 | 197 |
| 288285 | 12 | 663 | CCTCGAAGTCAGAGTCATTG | 82 | 198 |
| 288286 | 12 | 668 | CACCACCTCGAAGTCAGAGT | 81 | 199 |
| 288287 | 12 | 673 | TGGTACACCACCTCGAAGTC | 74 | 200 |
| 288288 | 12 | 678 | ATTGCTGGTACACCACCTCG | 75 | 201 |

### Example 5

### Antisense Inhibition of Rat LMW-PTPase Expression by Oligomeric Compounds

Antisense compounds were designed against one or more of the rat LMW-PTPase target nucleic acid sequences cited in Table 1 and were screened in vitro to determine the compound's ability to modulate expression of a target nucleic acid that encodes LMW-PTPase. The compounds shown in Table 7 are all chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the primer-probe set designed to hybridize to rat LMW-PTPase (Table 2). Data are averages from two experiments in which A10 cells were treated with 50 nM of the disclosed oligomeric compounds using LIPOFECTIN™. A reduction in expression is expressed as percent inhibition in Table 7. The control oligomeric compound used was SEQ ID NO: 25. If present, “N.D.” indicates “not determined”.

TABLE 7
Inhibition of rat LMW-PTPase mRNA levels by chimeric oligonucleotides having 2′-MOE wings
and deoxy gap

| | Target | | | | |
|---|---|---|---|---|---|
| Compound | SEQ ID | Target | | | SEQ ID |
| No. | NO | Site | Sequence (5′ to 3′) | % Inhib | NO |
| 288289 | 15 | 403 | ACCTCGAAGTCAGAGTCATT | 70 | 202 |
| 288233 | 16 | 24 | TGCCATGTTTCGGTGCAGAC | 74 | 153 |
| 288234 | 16 | 29 | ACCTCTGCCATGTTTCGGTG | 83 | 154 |
| 355621 | 16 | 36 | GGACCCAACCTCTGCCATGT | 89 | 203 |
| 288235 | 16 | 42 | TGACTTGGACCCAACCTCTG | 73 | 155 |
| 288238 | 16 | 57 | ACACACGAACAGCACTGACT | 29 | 158 |
| 288239 | 16 | 65 | TTACCGAGACACACGAACAG | 34 | 159 |
| 288241 | 16 | 72 | GCAAATGTTACCGAGACACA | 50 | 161 |
| 288274 | 16 | 308 | AGATTGCTTTCATCCATACA | 54 | 60 |
| 288286 | 16 | 444 | CACCACCTCGAAGTCAGAGT | 69 | 199 |
| 288287 | 16 | 449 | TGGTACACCACCTCGAAGTC | 81 | 200 |
| 288288 | 16 | 454 | ATTGCTGGTACACCACCTCG | 69 | 201 |
| 355622 | 16 | 461 | CTAAGGCATTGCTGGTACAC | 72 | 204 |
| 355623 | 16 | 466 | AGCACCTAAGGCATTGCTGG | 74 | 205 |
| 355624 | 16 | 471 | CTTGCAGCACCTAAGGCATT | 74 | 206 |
| 355625 | 16 | 476 | AAGGCCTTGCAGCACCTAAG | 83 | 207 |
| 355626 | 16 | 481 | CCAGGAAGGCCTTGCAGCAC | 86 | 208 |
| 355627 | 16 | 486 | CTTCTCCAGGAAGGCCTTGC | 79 | 209 |
| 355628 | 16 | 492 | GTGAGTCTTCTCCAGGAAGG | 82 | 210 |
| 355629 | 16 | 504 | TAGGACCAGCTAGTGAGTCT | 74 | 211 |
| 355630 | 16 | 519 | CTCAGTGGTGGTGGTTAGGA | 55 | 212 |
| 355631 | 16 | 556 | GCCACCACCCTTGGGCACAG | 78 | 213 |
| 355632 | 16 | 567 | GGCTAAGGACTGCCACCACC | 78 | 214 |
| 355633 | 16 | 607 | GATATACAGTAAGTCAGCTG | 80 | 215 |
| 355634 | 16 | 625 | ACCTACAATTATTTTAAAGA | 15 | 216 |
| 355635 | 16 | 633 | TGATTTCCACCTACAATTAT | 52 | 217 |
| 355636 | 16 | 638 | ATGCCTGATTTCCACCTACA | 91 | 218 |
| 355637 | 16 | 647 | TCTGAACAAATGCCTGATTT | 82 | 219 |
| 355638 | 16 | 674 | AATGTCTGCCTCAAATGTTT | 73 | 220 |
| 355639 | 16 | 680 | ACCTCAAATGTCTGCCTCAA | 78 | 221 |
| 355640 | 16 | 687 | GAGCCACACCTCAAATGTCT | 89 | 222 |
| 355641 | 16 | 700 | GTCTAAGAATACTGAGCCAC | 87 | 223 |
| 355642 | 16 | 706 | TTGTTAGTCTAAGAATACTG | 52 | 224 |
| 355643 | 16 | 725 | TATGGCGAGGCCAGAGCTTT | 77 | 225 |
| 355644 | 16 | 735 | ATTTTGTAATTATGGCGAGG | 60 | 226 |
| 355645 | 16 | 753 | ACAGTTGCTCGTTCCACTAT | 92 | 227 |
| 355646 | 16 | 759 | TGTTCCACAGTTGCTCGTTC | 95 | 228 |
| 355647 | 16 | 795 | CCTTGTGGGTCATTCTTACT | 71 | 229 |
| 355648 | 16 | 819 | GCTGGGCTCAAAGGCTGATC | 67 | 230 |
| 355649 | 16 | 841 | TTAGACCAGACTACCCAGGC | 80 | 231 |
| 355650 | 16 | 853 | CTCACACTCCAGTTAGACCA | 63 | 232 |
| 355651 | 16 | 871 | CACTGGGTGCTGGCCATGCT | 70 | 233 |
| 355652 | 16 | 890 | GTAAGGCAAGCAAACAGCAC | 40 | 234 |
| 355653 | 16 | 924 | TTGTCACAATAAGAGACAAT | 55 | 235 |
| 355654 | 16 | 931 | GGAGATATTGTCACAATAAG | 85 | 236 |
| 355655 | 16 | 939 | CCATGGATGGAGATATTGTC | 82 | 237 |
| 355656 | 16 | 944 | GGCTGCCATGGATGGAGATA | 79 | 238 |
| 355657 | 16 | 952 | AAATGGAAGGCTGCCATGGA | 80 | 239 |
| 355658 | 16 | 958 | AGTGTTAAATGGAAGGCTGC | 59 | 240 |
| 355659 | 16 | 970 | TTAAACTCTCCCAGTGTTAA | 76 | 241 |
| 355660 | 16 | 976 | CTGGGTTTAAACTCTCCCAG | 80 | 242 |
| 355661 | 16 | 1013 | GGTTCTCCTCTCTCAAATAT | 82 | 243 |
| 355662 | 16 | 1038 | AGTTCCAGGCCCATCATCAC | 61 | 244 |
| 355663 | 16 | 1050 | ATGGCCTGCTGGAGTTCCAG | 67 | 245 |
| 355664 | 16 | 1089 | TTTTTATCTTTCAGACAGGG | 19 | 246 |
| 355665 | 16 | 1103 | CCCATCTGTTAGCATTTTTA | 73 | 247 |
| 355666 | 16 | 1111 | CTGTTGCTCCCATCTGTTAG | 80 | 248 |
| 355667 | 16 | 1125 | TTAACTTCACCAACCTGTTG | 85 | 249 |
| 355668 | 16 | 1169 | CCAAGCTCAAGAAACTACAC | 69 | 250 |
| 355669 | 16 | 1187 | AAGTGGCTCAAATAGGAACC | 31 | 251 |
| 355670 | 16 | 1206 | CTTTCTCTTTAAAGAAGCAA | 24 | 252 |
| 355671 | 16 | 1215 | GCACACTTACTTTCTCTTTA | 84 | 253 |
| 355672 | 16 | 1228 | CACCACTATTTAAGCACACT | 28 | 254 |
| 355673 | 16 | 1237 | ACAAACGCACACCACTATTT | 62 | 255 |
| 355674 | 16 | 1255 | GTTGATGAGAGAACACTTAC | 79 | 256 |
| 355675 | 16 | 1270 | GTAACTTTGTAAAATGTTGA | 46 | 257 |
| 355676 | 16 | 1286 | TTACTCATGCTTGCCTGTAA | 90 | 258 |
| 355677 | 16 | 1312 | GTCCCTTTTCTGAAAATACA | 78 | 259 |
| 355678 | 16 | 1323 | ATAAATTTGAGGTCCCTTTT | 66 | 260 |
| 355679 | 16 | 1331 | ATATCCACATAAATTTGAGG | 68 | 261 |
| 355680 | 16 | 1345 | ATCTTTTCTGACATATATCC | 64 | 262 |
| 355613 | 17 | 3444 | TCTCCAGTGGCAAAGACAAA | 30 | 263 |
| 355614 | 17 | 5834 | AAGCAAGAAACTATGCGGGA | 45 | 264 |
| 355615 | 17 | 8275 | CATACGGTACCTGCCGTGCA | 53 | 265 |
| 355616 | 17 | 8312 | CAATGGCCCACTGTAACACA | 70 | 266 |
| 355617 | 17 | 13156 | GAGTACATTTGAAGTTAAAA | 45 | 267 |
| 355618 | 17 | 13309 | CTCTTGTAATCTACAATTAA | 3 | 268 |
| 355619 | 17 | 13459 | TATACCTGAGTTCAAGGTCA | 57 | 269 |
| 355620 | 18 | 204 | CTCTTGTAATCTGTCTTGCC | 27 | 270 |

### Example 6

### Inhibition of Mouse LMW-PTPase mRNA Levels by Chimeric Phosphorothioate Oligonucleotides Having 2′-MOE Wings and a Deoxy Gap:Dose Response Studies

In a further embodiment, six oligonucleotides were selected for dose-response studies: Compound No. 288285, Compound No. 288276, Compound No. 288268, Compound No. 288286, Compound No. 288267 and Compound No. 288291. Compound No. 129689 (GAGGTCTCGACTTACCCGCT, incorporated herein as SEQ ID NO: 271) and Compound No. 129695 (TTCTACCTCGCGCGATTTAC, incorporated herein as SEQ ID NO: 272, which are not targeted to LMW-PTPase, served as negative controls. Compound No. 129689 and Compound No. 129695 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-(2-methoxyethyl) (2′-MOE) nucleotides. The internucleoside (backbone) linkages are phosphorothioate (P═S) throughout the oligonucleotide. All cytidine residues are 5-methylcytidines.

Oligonucleotides were transfected into cells using the CYTOFECTIN™ Reagent (Gene Therapy Systems, San Diego, Calif.). b.END cells were treated with 12.5, 25, 50 or 100 nM of oligonucleotide. Untreated control cells served as the control to which data were normalized Quantitative real-time PCR to measure LMW-PTPase levels was performed as described herein.

Data were averaged from 3 experiments and the results are shown in Table 8 as percent inhibition relative to untreated control. Neither control oligonucleotide (Compound No. 129689 or Compound No. 129695) inhibited LMW-PTPase mRNA expression in this experiment.

TABLE 8
Inhibition of mouse LMW-PTPase mRNA expression
in mouse b.END cells: dose response

| | | |
|---|---|---|
| | | % Inhibition |
| Compound | SEQ ID | Dose of oligonucleotide (nM) |

| No. | NO | 12.5 | 25 | 50 | 100 |
|---|---|---|---|---|---|
| 288267 | 186 | 60 | 74 | 88 | 93 |
| 288268 | 187 | 54 | 72 | 80 | 93 |
| 288276 | 62 | 23 | 47 | 68 | 85 |
| 288285 | 198 | 11 | 41 | 67 | 86 |
| 288286 | 199 | 50 | 69 | 86 | 96 |
| 288291 | 133 | 72 | 82 | 90 | 92 |

As demonstrated in Table 8, Compound No. 288267, Compound No. 288268, Compound No. 288276, Compound No. 288285, Compound No. 288286 and Compound No. 288291 inhibited mouse LMW-PTPase mRNA expression in a dose-dependent manner

### Example 7

### Inhibition of Rat LMW-PTPase mRNA Levels by Chimeric Phosphorothioate Oligonucleotides Having 2′-MOE Wings and a Deoxy Gap:Dose Response Studies

In a further embodiment, seven oligonucleotides were selected for dose-response studies: Compound No. 355621, Compound No. 355636, Compound No. 355676, Compound No. 355626, Compound No. 355654, Compound No. 355640, and Compound No. 355641. Compound No. 15770, Compound No. 129690 (TTAGAATACGTCGCGTTATG, incorporated herein as SEQ ID NO: 273), and Compound No. 141923 (CCTTCCCTGAAGGTTCCTCC, incorporated herein as SEQ ID NO: 274), which are not targeted to LMW-PTPase, served as controls. Compound No. 129690 and Compound No. 141923 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Rat primary hepatocytes were treated with 12.5, 25, 50, 100 or 200 nM of oligonucleotide, using LIPOFECTIN™ as described herein. Untreated control cells served as the control to which data were normalized Treatment with the transfection mixture and quantitative real-time PCR to measure rat LMW-PTPase levels were both performed as described herein.

Results of these studies are shown in Table 9. Data are averaged from four experiments and are expressed as percent inhibition relative untreated control. None of the control oligonucleotides tested (Compound No. 141923, Compound No. 15770 or Compound No. 129690) resulted greater than 4% inhibition of rat LMW-PTPase; data from cells treated with Compound No. 15770 is shown in Table 8 and is representative of the control oligonucleotide treatments.

TABLE 9
Inhibition of rat LMW-PTPase mRNA expression in
rat primary hepatocyte cells: dose response

| | | % Inhibition | | | | |
|---|---|---|---|---|---|---|
| Compound | SEQ ID | Dose of oligonucleotide (nM) | | | | |
| No. | NO | 12.5 | 25 | 50 | 100 | 200 |
| 355621 | 203 | 17 | 29 | 52 | 70 | 82 |
| 355626 | 208 | 6 | 17 | 33 | 48 | 70 |
| 355636 | 218 | 17 | 27 | 43 | 64 | 80 |
| 355640 | 222 | 19 | 31 | 55 | 73 | 84 |
| 355641 | 223 | 14 | 31 | 46 | 65 | 80 |
| 355654 | 236 | 18 | 25 | 42 | 66 | 81 |
| 355676 | 258 | 15 | 24 | 49 | 62 | 77 |
| 15770 | 31 | 0 | 1 | 0 | 4 | 0 |

As demonstrated in Table 9, Compound No. 355621, Compound No. 355626 Compound No. 355636, Compound No. 355640, Compound No. 355641, Compound No. 355654 and Compound No. 355676 inhibited LMW-PTPase mRNA expression in a dose-dependent manner

### Example 8

### Antisense Inhibition of LMW-PTPase Expression In Vivo: Ob/Ob Mice

Leptin is a hormone produced by fat that regulates appetite. Deficiencies in this hormone in both humans and non-human animals leads to obesity. ob/ob mice have a mutation in the leptin gene which results in obesity and hyperglycemia. As such, these mice are a useful model for the investigation of obesity and diabetes and treatments designed to treat these conditions. ob/ob mice have higher circulating levels of insulin and are less hyperglycemic than db/db mice, which harbor a mutation in the leptin receptor. In accordance with the present invention, the oligomeric compounds of the invention are tested in the ob/ob model of obesity and diabetes.

C57B1/6J-Lep ob/ob mice (Jackson Laboratory, Bar Harbor, Me.) are subcutaneously injected with Compound No. 288267 (SEQ ID NO: 186) at a dose of 25 mg/kg two times per week for 4 weeks (n=5). Saline-injected animals serve as controls (n=4). After the treatment period, mice are sacrificed and target levels were evaluated in liver and in fat. RNA isolation and target mRNA expression level quantitation were performed as described by other examples herein Animals treated with Compound No. 288267 on average showed 90% reduction in liver LMW-PTPase levels as compared to saline treated control animals. LMW-PTPase mRNA levels in epididymal fat were reduced 70% on average in animals treated with Compound No. 288267.

To assess the physiological effects resulting from inhibition of target mRNA, the ob/ob mice were evaluated at the end of the treatment period (day 28) for serum triglycerides and serum glucose levels. These parameters were measured by routine clinical analyzer instruments (e.g. Olympus Clinical Analyzer, Melville, N.Y.). At day 28, the average triglyceride levels measured for saline-treated control animals was 168 mg/dL, while the average for animals treated with Compound No. 288267 was 75 mg/dL. At day 28, glucose was 491 mg/dL for animals treated with saline alone and 258 mg/dL for animals treated with Compound No. 288267. Therefore, treatment with Compound No. 288267 caused substantial decreases in glucose and in triglyceride levels. Therefore, one embodiment of the present invention is a method of lowering glucose by administering an oligomeric compound of the invention, and another embodiment of the present invention is a method of lowering triglycerides by administering an oligomeric compound of the invention. In one embodiment, the triglycerides are blood, plasma, or serum triglycerides. Another embodiment of the current invention is a method of ameliorating or lessening the severity of a condition in an animal. In some embodiments, the condition is diabetes. In some embodiments, the diabetes is type II diabetes. In other embodiments, the condition is metabolic syndrome.

To further assess the effects of inhibition of target mRNA on glucose metabolism, fasted serum glucose was measured via routine clinical analysis. The average fasted serum glucose level for animals treated with Compound No. 288267 was 194 mg/dL, while the average fasted level measured for animals treated with saline alone was 330 mg/dL. Therefore, another embodiment of the present invention is a method of lowering serum glucose.

Insulin levels were also measured after four weeks of treatment using a commercially available kit (e.g. Alpco insulin-specific ELISA kit, Windham, N.H.). Treatment with Compound No. 288267 caused about a 45% reduction in circulating plasma insulin levels. Decreased insulin levels can indicate improvement in insulin sensitivity. In one embodiment, the present invention provides methods of improving insulin sensitivity. In a further embodiment, decreased insulin levels are indicative of improved insulin sensitivity.

At the end of the study, mice were sacrificed and tissues were weighed. The average liver and spleen weights were not substantially altered by treatment with Compound No. 288267 as compared to saline-treated controls. Epididymal white adipose tissue weight was reduced by about 10% in animals treated with Compound No. 288267. Therefore, another embodiment of the present invention is a method of reducing adiposity in an animal by administering an oligomeric compound of the invention.

### Example 9

### Effect of Antisense Inhibition of LMW-PTPase on Insulin Receptor Phosphorylation in Ob/Ob Mice

To assess the effects of inhibition of LMW-PTPase on receptor phosphorylation, a bolus of insulin (2 U/kg) was administered about 8 to 9 minutes prior to sacrifice to a group of the ob/ob mice treated with Compound No. 288267 (SEQ ID NO: 186) or saline as described in Example 8. Liver samples were pooled and subjected to standard immunoprecipitation and Western blot procedures and analyses. Briefly, tissues were lysed in lysis buffer (150 mM NaCl, 50 mM Tris, pH 7.5, 1% Triton X-100, 0.5% NP-40, 0.25% Nadeoxycholate, 1 mM EDTA, 1 mM EGTA, 1 mM NaOV, 1 mM NaF, and protease inhibitor cocktail I (Calbiochem). The lysates were clarified by centrifugation for 15 min at 12000 g. The clarified lysates were first incubated with protein agarose A/G beads (1:1 ratio) for 3-4 h at 4.deg.C. followed by incubation with anti-phosphotyrosine antibody for another 3-4-h at 4.deg.C. The immune complex was then washed with lysis buffer, boiled in Laemmli's sample buffer, and analyzed by Western blot. The membrane was blotted with a commercially available anti-insulin receptor beta (IR-.beta. subunit antibody (Santa Cruz, Calif.). The signal was detected by using a commercially available HRP-conjugated goat anti-rabbit IgG antibody and ECL.

Quantitation of the resulting bands showed approximately a 4 to 6-fold increase in phosphorylation of the insulin receptor upon insulin stimulation in the samples from animals treated with Compound No. 288267 as compared to that measured for saline-treated controls. These data suggest improved insulin sensitivity upon treatment with antisense oligonucleotides targeted to LMW-PTPase.

### Example 10

### Effect of Antisense Inhibition of LMW-PTPase on PI3-K Activity in Ob/Ob Mice

To further assess the role of LMW-PTPase in the insulin receptor signaling pathway and to assess the effects of antisense inhibition of LMW-PTPase on insulin action, clarified lysates prepared as described in Example 11 from livers or fat tissue of ob/ob mice treated as described in Examples 8 and 9, and were subjected to further analyses.

Phosphatidyl inositol 3-kinase (PI3-K) is an enzyme activated downstream of insulin-receptor stimulation. PI3-kinase activity was measured using methods known in the art (Pandey et al., Biochemistry, 1998, 37, 7006-7014). Briefly, the clarified lysates were subjected to immunoprecipitation with IRS-1/2 antibody (1 ug) for 2 h at 4.deg.C., followed by incubation with protein A/G sepharose for an additional 2 h. The immune complexes were washed and subjected to in vitro PI3-Kinase assay using L-.alpha.-phosphatidylinositol (PI) as an exogenous substrate. The phosphorylated lipid was isolated and separated by thin-layer chromatography (TLC). The TLC plate was exposed to Kodak film, and the radioactive spots associated with the product of PI3-K activity (PIP2) was scratched off of the TLC plates and counted in a scintillation counter. Average results from the scintillation counts are shown in Table 10 in arbitrary units for the livers from each treatment group with or without insulin. The antibody used for the immunoprecipitation is shown in the column designated “IP” (IRS-1 or IRS-2).

TABLE 10
Effects of antisense inhibition of LMW-PTPase
on PI3-K activity in ob/ob mouse liver

PI3-K activity
(arbitrary units)

| Treatment group | IP | −Insulin | +Insulin |
|---|---|---|---|
| Saline | IRS-1 | 260853 | 257907 |
| Compound No. 288267 | IRS-1 | 291982 | 674202 |
| Saline | IRS-2 | 596 | 677 |
| Compound No. 288267 | IRS-2 | 679 | 1092 |

As shown in Table 10, Compound No. 288267 (SEQ ID NO: 186) caused increases in insulin-stimulated PI3-K activity above the increases observed for animals treated with saline.

Approximate results from the scintillation counts are shown in Table 11 as averages in arbitrary units for adipose tissue from each treatment group with or without insulin. The antibody used for the immunoprecipitation was IRS-1.

TABLE 11
Effects of antisense inhibition of LMW-PTPase
on PI3-K activity in ob/ob mouse fat tissue

PI3-K activity
(arbitrary units)

| Treatment group | −Insulin | +Insulin |
|---|---|---|
| Saline | 319 | 365 |
| Compound No. 288267 | 438 | 725 |

As shown in Table 11, Compound No. 288267 caused increases in insulin-stimulated PI3-K activity above the increases observed for animals treated with saline. Taken together, these results demonstrate a novel role for LMW-PTPase in insulin action and show that antisense inhibition of LMW-PTPase improves insulin signaling in ob/ob mice. Therefore another embodiment of the present invention is a method of improving insulin signaling in an animal by administering an oligomeric compound of the invention.

### Example 11

### Effect of Antisense Inhibition of LMW-PTPase on Insulin Signaling: In Vitro Studies

In accord with the present invention, the effects of antisense oligonucleotides targeted to LMW-PTPase on insulin-signaling were investigated in primary hepatocytes cultured from ob/ob mice using methods described herein. The ob/ob primary hepatocytes were treated with Compound No. 288267 (SEQ ID NO: 186) or the control oligonucleotide Compound No. 141923 (SEQ ID NO: 274) by transfection methods described herein. Treated cells were incubated for 10 minutes in the absence or presence of 100 nM insulin. Cells treated with transfection reagent alone served as controls.

Cell lysates were prepared and subjected to immunoprecipitation with anti-phosphotyrosine antibody followed by Western blot analysis using an anti-IR-.beta. antibody (Santa Cruz, Calif.) via standard methods. Cells treated with Compound No. 288267 showed a larger increase in insulin-stimulated IR-.beta. phosphorylation than was observed for control cells or cells treated with Compound No. 141923.

In a similar experiment using a commercially available antibody which recognizes phosphorylated Akt (Cell Signaling, Boston, Mass.), cells treated with Compound No. 288267 showed a larger increase in insulin-stimulated Akt phosphorylation than was observed for control cells or cells treated with Compound No. 141923. These data demonstrate that LMW-PTPase plays a role in the insulin signaling pathway.

Antisense compound modulation of LMW-PTPase expression levels was determined using primary mouse hepatocytes by treating the cells with 100 ng of Compound No. 288267 or with vehicle as described above. Following incubation, the cells were lysed in RTL buffer and total RNA was isolated using QIAGEN RNA easy kits (Qiagen, Valencia, Calif.). RT-PCR was performed as described above. These data showed an approximate 90% reduction in LMW-PTPase mRNA levels compared to control. A corresponding reduction in LMW-PTPase protein levels was seen by western blot analysis of the LMW-PTPase protein. Interestingly, there was no significant reduction in PTP1b levels in the presence of Compound No. 288267 compared to control cells (antibody available from Upstate Cell Signaling Solutions).

To further determine the action of LMW-PTPase antisense compounds on the insulin signaling pathway duplicate cell culture well comprising primary mouse hepatocytes were treated with 100 ng of either Compound No. 288267, Compound No. 288291, control Compound No. 141923, an antisense inhibitor of PTP1b or a combination of Control No. 288291 and the antisense inhibitor of PTP1b. A saline treated control was used as well. For each treatment group, one of the wells was incubated with 5 nM of insulin for 10 minutes. Following incubation the cell were analyzed for levels of phosphorylated IR-.beta., phosphoAkt, unphosphorylated Akt, PTP1b or LMW-PTPase using western blot techniques. These data show that IR-.beta. is phosphorylated in the presence of LMW-PTPase antisense compounds, but not in the presence of PTP-1b antisense compounds either alone or combined with the LMW-PTPase antisense compound. Western blot techniques are well known to those of ordinary skill in the art and antibodies are readily available from a number of commercial vendors (e.g., Upstate Cell Signaling Solutions, Charlottesville, Va.). (Harlow, E. and Lane, D., Antibodies: A Laboratory Manual, (1988) Cold Spring Harbor Press).

### Example 12

### Design of Oligomeric Compounds Targeting Human LMW-PTPase

A series of oligomeric compounds was designed to target different regions of human LMW-PTPase, using published sequences cited in Table 1. The compounds are shown in Table 12. All compounds in Table 12 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines.

TABLE 12
Chimeric oligonucleotides having 2′-MOE wings and deoxy gap targeting human LMW-PTPase

| | Target | | | |
|---|---|---|---|---|
| Compound | SEQ ID | Target | | SEQ ID |
| No. | NO | Site | Sequence (5′ to 3′) | NO |
| 105809 | 1 | 1 | ACCCCGTTCCGCACGCCCCC | 279 |
| 105811 | 1 | 41 | TAGCCTGTTCCGCCATCTTC | 280 |
| 105812 | 1 | 241 | GCTCATGGGAATGCCGTGCC | 281 |
| 105813 | 1 | 271 | ATCTTCTTTGGTAATCTGCC | 282 |
| 105814 | 1 | 421 | ATAGGGATCTTCAATAATAA | 283 |
| 105815 | 1 | 451 | CACCGTCTCAAAGTCAGAGT | 284 |
| 105816 | 1 | 571 | CCGACTGAGAAATGCAGGAC | 285 |
| 105817 | 1 | 611 | AACAAAGAGCTGGCTTTGGG | 286 |
| 105818 | 1 | 671 | CAAACACAACTGATTTCCAT | 287 |
| 105819 | 1 | 701 | TGAATCAAACATTTTTATTG | 288 |
| 105820 | 1 | 781 | TTGTTCTACTATTTTTGTAA | 289 |
| 105821 | 1 | 811 | GTGAGGTTTTCCTTCATTGT | 290 |
| 105822 | 1 | 961 | ACTACTGTCAATCCACAAAA | 291 |
| 105823 | 1 | 1051 | TCTTCCCTATCTTTTCAATA | 292 |
| 105824 | 1 | 1091 | GTATTGAAGGTGCCAACGAC | 293 |
| 105825 | 1 | 1171 | TAAGTTTCAGAGGCAAAGTG | 294 |
| 105826 | 1 | 1201 | CATACAAGTGTCCTTCTTTC | 295 |
| 105827 | 1 | 1291 | TTATTTTAAAAAATAAGCCA | 296 |
| 105828 | 1 | 1321 | AAATAATAACACTTTTCCCA | 297 |

### Example 13

### Design of Oligomeric Compounds Targeting Mouse LMW-PTPase

A series of oligomeric compounds was designed to target different regions of mouse LMW-PTPase, using published sequences cited in Table 1. The compounds are shown in Table 13. All compounds in Table 13 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines.

TABLE 13
Chimeric oligonucleotides having 2′-MOE wings and deoxy gap targeting mouse LMW-PTPase

| | Target | | | |
|---|---|---|---|---|
| Compound | SEQ ID | Target | | SEQ ID |
| No. | NO | Site | Sequence (5′ to 3′) | NO |
| 349037 | 11 | 246 | AGGTTTAGTTAGTCTAAGAA | 298 |
| 349038 | 11 | 248 | AGAGGTTTAGTTAGTCTAAG | 299 |
| 349039 | 11 | 250 | TCAGAGGTTTAGTTAGTCTA | 300 |
| 349040 | 11 | 254 | AAGGTCAGAGGTTTAGTTAG | 301 |
| 349041 | 11 | 256 | GCAAGGTCAGAGGTTTAGTT | 302 |
| 349042 | 11 | 258 | CCGCAAGGTCAGAGGTTTAG | 303 |
| 349043 | 11 | 296 | TTTGTTCCATATTTGCTTGT | 304 |
| 349044 | 12 | 307 | ATGGGTGACCGGCAAATGTT | 305 |
| 349045 | 12 | 308 | AATGGGTGACCGGCAAATGT | 306 |
| 349046 | 12 | 311 | TGCAATGGGTGACCGGCAAA | 307 |
| 349047 | 12 | 312 | CTGCAATGGGTGACCGGCAA | 308 |
| 349048 | 12 | 313 | TCTGCAATGGGTGACCGGCA | 309 |
| 349049 | 12 | 314 | TTCTGCAATGGGTGACCGGC | 310 |
| 349050 | 12 | 315 | CTTCTGCAATGGGTGACCGG | 311 |
| 349051 | 12 | 316 | GCTTCTGCAATGGGTGACCG | 312 |
| 349052 | 12 | 318 | CTGCTTCTGCAATGGGTGAC | 313 |
| 349053 | 12 | 320 | TACTGCTTCTGCAATGGGTG | 314 |
| 349054 | 12 | 322 | AATACTGCTTCTGCAATGGG | 315 |
| 349055 | 12 | 327 | TCCTGAATACTGCTTCTGCA | 316 |
| 349056 | 12 | 330 | GTTTCCTGAATACTGCTTCT | 317 |
| 349057 | 12 | 332 | CAGTTTCCTGAATACTGCTT | 318 |
| 349058 | 12 | 335 | TACCAGTTTCCTGAATACTG | 319 |
| 349059 | 12 | 337 | GTTACCAGTTTCCTGAATAC | 320 |
| 349060 | 12 | 339 | CAGTTACCAGTTTCCTGAAT | 321 |
| 349061 | 11 | 477 | TGTATGCTCGCTCCTCCTCT | 322 |
| 349062 | 12 | 503 | ATCGAATGTGGCAAAGTCTT | 323 |
| 349063 | 11 | 506 | CCGGCGCGCTCGCTCCGTCT | 324 |
| 349064 | 12 | 507 | TATAATCGAATGTGGCAAAG | 325 |
| 349065 | 12 | 509 | TATATAATCGAATGTGGCAA | 326 |
| 349066 | 12 | 512 | TAGTATATAATCGAATGTGG | 327 |
| 349067 | 12 | 513 | ATAGTATATAATCGAATGTG | 328 |
| 349068 | 12 | 515 | ACATAGTATATAATCGAATG | 329 |
| 349069 | 12 | 517 | ATACATAGTATATAATCGAA | 330 |
| 349070 | 12 | 531 | GATTGCTTTCATCCATACAT | 331 |
| 349071 | 12 | 533 | CAGATTGCTTTCATCCATAC | 332 |
| 349072 | 12 | 536 | TCTCAGATTGCTTTCATCCA | 333 |
| 349073 | 11 | 537 | TTGTCGCCTCCCGCGTCGTG | 334 |
| 349074 | 12 | 539 | ATCTCTCAGATTGCTTTCAT | 335 |
| 349075 | 12 | 541 | AGATCTCTCAGATTGCTTTC | 336 |
| 349076 | 12 | 543 | TGAGATCTCTCAGATTGCTT | 337 |
| 349077 | 11 | 574 | CTCCTGCCACATGTAGTCCG | 338 |
| 349078 | 11 | 643 | CTGCTCGGGCCCTTATTTTC | 339 |
| 349079 | 12 | 665 | CACCTCGAAGTCAGAGTCAT | 340 |
| 349080 | 12 | 667 | ACCACCTCGAAGTCAGAGTC | 341 |
| 349081 | 12 | 669 | ACACCACCTCGAAGTCAGAG | 342 |
| 349082 | 12 | 670 | TACACCACCTCGAAGTCAGA | 343 |
| 349083 | 12 | 671 | GTACACCACCTCGAAGTCAG | 344 |
| 349084 | 12 | 672 | GGTACACCACCTCGAAGTCA | 345 |
| 349085 | 12 | 674 | CTGGTACACCACCTCGAAGT | 346 |
| 349086 | 12 | 675 | GCTGGTACACCACCTCGAAG | 347 |
| 349087 | 12 | 676 | TGCTGGTACACCACCTCGAA | 348 |
| 349088 | 11 | 676 | TACGACCGCGACGGCCGCGT | 349 |
| 349089 | 12 | 677 | TTGCTGGTACACCACCTCGA | 350 |
| 349090 | 11 | 745 | CGAAGGTGTTCGTGTTACTC | 351 |
| 349091 | 11 | 824 | GCGTTGGCGCGTCGTCGCTG | 352 |
| 349092 | 11 | 876 | GGGTGGTAGTGGCGGGTGGG | 353 |
| 349093 | 11 | 931 | GTGGCTGGGTGGTGTCGTGT | 354 |

### Example 14

### Antisense Inhibition of LMW-PTPase Expression In Vivo: Mouse Model of Diet-Induced Obesity

The C57BL/6 mouse strain is reported to be susceptible to hyperlipidemia-induced atherosclerotic plaque formation. When these mice are fed a high-fat diet, they develop diet-induced obesity. Accordingly these mice are a useful model for the investigation of obesity and treatments designed to treat this conditions. In a further embodiment of the present invention, the oligomeric compounds of the invention are tested in a model of diet-induced obesity.

Male C57BL/6 mice received a 60% fat diet for about 12-13 weeks, after which mice were subcutaneously injected with Compound No. 288267 (SEQ ID NO: 186), an antisense oligonucleotide targeted to LMW-PTPase, or the control compound Compound No. 141923 (SEQ ID NO: 274) at a dose of 25 mg/kg two times per week for 6 weeks. Each treatment group was comprised of about 8 to 10 animals Saline-injected high-fat fed animals serve as a control. As an additional control, mice fed a normal chow diet were treated with saline alone.

Body weight and accumulated food intake were measured throughout the study for each treatment group. No significant alterations in accumulated food intake were observed for the animals fed a high-fat diet, regardless of the treatment. At the end of the study, body composition was assayed by MRI. Treatment with Compound No. 288267 caused about a 12% decrease in fat content of the high-fat fed mice over the treatment period. The fat content of high-fat fed animals treated with saline alone or with the control compound Compound No. 141923 did not decrease over the course of the study. Therefore, another embodiment of the present invention is a method of reducing adiposity in an animal by administering an oligomeric compound targeted to LMW-PTPase.

To assess the physiological effects resulting from inhibition of target mRNA, the diet-induced obese mice that received treatment were further evaluated at beginning of the study (week 0), during the 3rd week of treatment (week 3.5), and at the end of the treatment period (week 6) for plasma triglyceride and plasma cholesterol levels. Triglycerides and cholesterol are measured by routine clinical analyzer instruments (e.g. Olympus Clinical Analyzer, Melville, N.Y.). Average results for each treatment group are shown in Table 14 in mg/dL.

TABLE 14
Effects of antisense inhibition of
LMW-PTPase on plasma lipid levels

| | Cholesterol | | | Triglycerides | | |
|---|---|---|---|---|---|---|
| | Week | Week | Week | Week | Week | Week |
| Treatment | 0 | 3.5 | 6 | 0 | 3.5 | 6 |
| Saline, high-fat fed | 176 | 175 | 184 | 83 | 65 | 76 |
| Compound No. 141923 | 182 | 174 | 181 | 78 | 70 | 59 |
| Compound No. 288267 | 176 | 157 | 136 | 79 | 60 | 56 |
| Saline, normal diet | 82 | 68 | 68 | 74 | 76 | 61 |

As shown in Table 14, Treatment with Compound No. 288267 caused a decrease in plasma cholesterol in diet-induced obese mice. Therefore embodiments of the present invention include methods of lowering cholesterol in an animal by administering an oligomeric compound of the invention.

The effects of target inhibition on glucose and insulin metabolism were also evaluated in the diet-induced obese mice treated with the oligomeric compounds of the invention. Plasma glucose was measured at beginning of the study (week 0), during the 3rd week of treatment (week 3.5), and at the end of the treatment period (week 6). Plasma insulin was similarly measured at beginning of the study (week 0), during the 3rd week of treatment (week 3.5), and at the end of the treatment period (week 6). Glucose levels were measured using standard methods (for example, with a YSI glucose analyzer, YSI Scientific, Yellow Springs, Ohio) and insulin levels were measured using a commercially available kit (for example, an Alpco insulin-specific ELISA kit, Windham, N.H.). Hypoglycemia was not observed in animals treated with Compound No. 288267. Insulin levels are shown in Table 15 as a percentage of insulin levels measured for high-fat fed animals treated with saline alone.

TABLE 15
Effects of antisense inhibition of LMW-PTPase
on blood glucose and insulin levels

| | Insulin | | |
|---|---|---|---|
| Treatment | Week 0 | Week 3.5 | Week 6 |
| Saline, high-fat fed | 100 | 114 | 179 |
| Compound No. 141923 | 100 | 107 | 164 |
| Compound No. 288267 | 100 | 86 | 100 |
| Saline, normal diet | 21 | 86 | 79 |

As shown in Table 15, treatment with Compound No. 288267 prevented the increase in insulin levels over the course of the study observed in high-fat fed animals treated with saline or with the control compound Compound No. 141923. Therefore, another embodiment of the present invention is a method of improving insulin sensitivity in an animal by administering an oligomeric compound targeted to LMW-PTPase. In one embodiment, improved insulin sensitivity is indicated by a reduction in circulating insulin levels.

Glucose tolerance tests were also administered in fasted mice. During the fifth week of treatment, mice were fasted overnight and then received intraperitoneal injections of glucose at a dose of 1 g/kg, and the blood glucose were measured before the glucose challenge and at 30 minute intervals for up to 2 hours. Results are shown in Table 16 for each treatment group.

TABLE 16
Effects of antisense inhibition of
LMW-PTPase on glucose tolerance

| | Glucose (mg/dL) | | | | |
|---|---|---|---|---|---|
| | 0 | 30 | 60 | 90 | 120 |
| Treatment | min. | min. | min. | min. | min. |
| Saline, high-fat fed | 110 | 346 | 264 | 219 | 194 |
| Compound No. 141923 | 112 | 317 | 243 | 210 | 185 |
| Compound No. 288267 | 118 | 277 | 210 | 188 | 168 |
| Saline, normal diet | 100 | 249 | 184 | 151 | 133 |

A plot of the data in Table 16 as glucose level as a function of time allows for comparison of the area under the curve for each treatment group. These data reveal improved glucose tolerance in high-fat fed animals treated with Compound No. 288267. Therefore, another aspect of the present invention is a method of improving glucose tolerance in an animal by administering an oligomeric compound of the invention.

Insulin tolerance tests were also administered in fasted mice. During the fourth week of treatment, mice were fasted for approximately 4 to 5 hours, and then received intraperitoneal injections of insulin at a dose of 0.5 U/kg. Glucose levels were measured before the insulin challenge and at 30 minute intervals for up to 2 hours. Results are shown in Table 17.

TABLE 17
Effects of antisense inhibition of
LMW-PTPase on insulin tolerance

| | Glucose (mg/dL) | | | | |
|---|---|---|---|---|---|
| | 0 | 30 | 60 | 90 | 120 |
| Treatment | min. | min. | min. | min. | min. |
| Saline, high-fat fed | 218 | 122 | 123 | 129 | 154 |
| Compound No. 141923 | 208 | 127 | 108 | 116 | 145 |
| Compound No. 288267 | 172 | 104 | 87 | 88 | 117 |

A plot of the data in Table 17 as glucose level as a function of time allows for comparison of the area under the curve for each treatment group. These data reveal improved insulin tolerance in high-fat fed animals treated with Compound No. 288267. Therefore, another aspect of the present invention is a method of improving insulin tolerance in an animal by administering an oligomeric compound of the invention.

### Example 15

### Antisense Inhibition of LMW-PTPase Expression in a Mouse Model of Diet-Induced Obesity

Liver and fat tissues from C57BL/6 mice fed a high fat diet and treated as described in Example 14 were further evaluated at the end of the study for target reduction. Analysis of target reduction in tissues was performed as described herein. Treatment with Compound No. 288267 reduced LMW-PTPase gene expression by about 90% in liver and about 75% in fat (white adipose tissue), whereas treatment with Compound No. 141923 did not reduce LMW-PTPase expression in either tissue. Treatment with Compound No. 288267 also caused a significant reduction in hepatic glucose-6-phosphatase expression, suggesting a suppression of hepatic glucose output. Treatment with Compound No. 288267 did not have an effect on the expression of SHP2, SHPTP2 or PTEN expression. Similar results are seen with ob/ob mice. Therefore, another embodiment of the present invention is a method of reducing hepatic glucose output in an animal by administering an oligomeric compound of the invention. Another embodiment of the present invention is a method of modulating genes involved in glucose metabolism by administering an oligomeric compound of the invention. In one embodiment, the modulated gene is glucose-6-phosphatase.

Liver triglyceride levels were also assessed, using a commercially available kit (for example, using the Triglyceride GPO Assay from Roche Diagnostics, Indianapolis, Ind.). Liver triglyceride content was reduced with Compound No. 288267 treatment as compared to treatment with Compound No. 141923 (about 35 mg/g vs. about 56 mg/g tissue, respectively).

Hepatic steatosis may also be assessed by routine histological analysis of frozen liver tissue sections stained with oil red 0 stain, which is commonly used to visualize lipid deposits, and counterstained with hematoxylin and eosin, to visualize nuclei and cytoplasm, respectively.

Therefore, another embodiment of the present invention is a method of decreasing hepatic triglyceride accumulation in an animal by administering an oligomeric compound of the invention. Another embodiment of the present invention is a method of treating steatosis in an animal by administering an oligomeric compound of the invention. In one embodiment, the steatosis is steatohepatitis. In one embodiment, the steatosis is NASH.

Another embodiment of the present invention is a method of treating diabetes or metabolic syndrome comprising administering an oligomeric compound of the invention. In some embodiments, the oligomeric compound inhibits LMW-PTPase expression in the liver, in fat, or in both tissues.

What is claimed is:

1. An antisense compound 15 to 35 nucleobases in length targeted to and hybridizable with a nucleic acid molecule encoding LMW-PTPase, wherein the antisense compound comprises a modified sugar and is capable of inhibiting expression of LMW-PTPase.

2. The antisense compound of claim 1, wherein the antisense compound further comprises at least one modified internucleoside linkage or at least one modified nucleobase.

3. The antisense compound of claim 2, wherein the modified internucleoside linkage is a phosphorothioate linkage.

4. The antisense compound of claim 1, wherein the modified sugar moiety is a high affinity modification comprising a 2′-O-(2-methoxyethyl), a 2-O-methyl, an LNA or an ENA.

5. The antisense compound of claim 1, wherein the antisense compound is chimeric and comprises deoxynucleotides in a first region, at least one high affinity modified sugar in each of a second region and a third region, which flank the first region on the 5′ end and the 3′ end, respectively, and at least one phosphorothioate modified internucleoside linkage.

6. The antisense compound of claim 5, wherein the first region is ten deoxynucleotides in length, the second and third regions are each five nucleotides in length and comprise five 2′-O-(2-methoxyethyl) nucleotides, and each internucleoside linkage in the chimeric oligonucleotide is a phosphorothioate.

7. The antisense compound of claim 1, wherein the antisense compound is targeted to at least a 12 nucleobase portion of an active target segment of SEQ ID NO: 5, wherein the active target segment is selected from the group consisting of Region BA, Region BB, Region BC, Region BD, Region BE, Region BF, Region BG, Region BH, Region BI, Region BJ and Region BK.

8. The antisense compound of claim 7, wherein the antisense compound is targeted to at least a 20 nucleobase portion of an active target segment of SEQ ID NO: 5, wherein the active target segment is selected from the group consisting of Region BA, Region BB, Region BC, Region BD, Region BE, Region BF, Region BG, Region BH, Region BI, Region BJ and Region BK.

9. The antisense compound of claim 7, wherein the antisense compound further comprises at least one modified internucleoside linkage or at least one modified nucleobase.

10. The antisense compound of claim 7, wherein the modified internucleoside linkage is a phosphorothioate linkage.

11. The antisense compound of claim 7, wherein the modified sugar moiety is a high affinity modification comprising a 2′-O-(2-methoxyethyl), a 2-O-methyl, an LNA or an ENA.

12. The antisense compound of claim 7, wherein the antisense compound is chimeric and comprises deoxynucleotides in a first region, at least one high affinity modified sugar in each of a second region and a third region, which flank the first region on the 5′ end and the 3′ end, respectively, and at least one phosphorothioate modified internucleoside linkage.

13. The antisense compound of claim 7, wherein the antisense compound further comprises at least one modified internucleoside linkage or at least one modified nucleobase.

14. The antisense compound of claim 8, wherein the modified internucleoside linkage is a phosphorothioate linkage.

15. The antisense compound of claim 8, wherein the modified sugar moiety is a high affinity modification comprising a 2′-O-(2-methoxyethyl), a 2-O-methyl, an LNA or an ENA.

16. The antisense compound of claim 8, wherein the antisense compound is chimeric and comprises deoxynucleotides in a first region, at least one high affinity modified sugar in each of a second region and a third region, which flank the first region on the 5′ end and the 3′ end, respectively, and at least one phosphorothioate modified internucleoside linkage.

17. A method of lowering triglyceride levels, improving insulin sensitivity, lowering blood glucose levels, lowering cholesterol, or improving glucose tolerance in an animal comprising administering the antisense compound of claim 1 to the animal, thereby lowering triglyceride levels, improving insulin sensitivity, lowering blood glucose levels, lowering cholesterol, or improving glucose tolerance in the animal.

18. The method of claim 17, wherein the triglyceride levels are blood, plasma, or serum triglyceride levels.

19. The method of claim 17, wherein the cholesterol is LDL cholesterol or VLDL cholesterol.

20. A method of treating diabetes, obesity, insulin resistance, insulin deficiency, or hypercholesterolemia in an animal comprising administering the antisense compound of claim 1 to the animal, thereby treating diabetes, obesity, insulin resistance, insulin deficiency, or hypercholesterolemia in the animal.


SEQUENCE LISTING (363 sequences)

SEQ ID NO: 1 - DNA, 1521 bp, Homo sapiens
gggggcgtgcggaacggggtgtctcggcgcctctgcgcgggaagatggcggaacaggctaccaagtccgtgctgtttgtgtgtctgggtaacatttgtcgatcacccattgcagaagcagttttcaggaaacttgtaaccgatcaaaacatctcagagaattggagggtagacagcgcggcaacttccgggtatgagatagggaacccccctgactaccgagggcagagctgcatgaagaggcacggcattcccatgagccacgttgcccggcagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagccagctctttgttcagttgacttactgtttcttaccttaaaaagtaattgtagatggaaatcagttgtgtttggcaggagaatcaataaaaatgtttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggaggcaggtgcttgctccccagtgctacctctctcttccctagggccttttgtggattgacagtagtcccctccgtagagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaaaaaaaaaaaaaaaaaaaaaaaaaaccc

SEQ ID NO: 2 - DNA, 1468 bp, Homo sapiens
caggctaccaagtccgtgctgtttgtgtgtctgggtaacatttgtcgatcacccattgcagaagcagttttcaggaaacttgtaaccgatcaaaacatctcagagaattgggtcattgacagcggtgctgtttctgactggaacgtgggccggtccccagacccaagagctgtgagctgcctaagaaatcatggcattcacacagcccataaagcaagacagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagccagctctttgttcagttgacttactgtttcttaccttaaaaagtaattgtagatggaaatcagttgtgtttggcaggagaatcaataaaaatgtttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggaggcaggtgcttgctccccagtgctacctctctcttccctagggccttttgtggattgacagtagtcccctccgtagagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaaaaaaaaaaaaaaaaaaaaaaaaaaccc

SEQ ID NO: 3 - DNA, 1549 bp, Homo sapiens
tggcagtgcgcctgcgccgcgtcggcgtgcggaacgccgcggtgtctcggcgcctctgcgcgcgggaagatggcggaacaggctaccaagtccgtgctgtttgtgtgtctgggtaacatttgtcgatcacccattgcagaagcagttttcaggaaacttgtaaccgatcaaaacatctcagagaattggagggtagacagcgcggcaacttccgggtatgagatagggaacccccctgactaccgagggcagagctgcatgaagaggcacggcattcccatgagccacgttgcccggcagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagcccagctctttgttcagttgacttactgtttcttaccttaaaaagtaattgtagatggaaatcagttgtgtttggcaggagaatcaataaaaatctttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggagggcaggtgccctctgctccccagtgctacctctctcttccctagggccttttgtggattgacagtagtcccctccgtaggagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaaaaaaaaaaaaaaaaaaaaaaaaaa

SEQ ID NO: 4 - DNA, 1549 bp, Homo sapiens
tggcagtgcgcctgcgccgcgtcggcgtgcggaacgccgcggtgtctcggcgcctctgcgcgcgggaagatggcggaacaggctaccaagtccgtgctgtttgtgtgtctgggtaacatttgtcgatcacccattgcagaagcagttttcaggaaacttgtaaccgatcaaaacatctcagagaattgggtcattgacagcggtgctgtttctgactggaacgtgggccggtccccagacccaagagctgtgagctgcctaagaaatcatggcattcacacagcccataaagcaagacagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagcccagctctttgttcagttgacttactgtttcttaccttaaaaagtaattgtagatggaaatcagttgtgtttggcaggagaatcaataaaaatctttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggagggcaggtgccctctgctccccagtgctacctctctcttccctagggccttttgtggattgacagtagtcccctccgtaggagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaaaaaaaaaaaaaaaaaaaaaaaaaa

SEQ ID NO: 5 - DNA, 1578 bp, Homo sapiens
tggcagtgcgcctgcgccgcgtcggcgtgcggaacgccgcggtgtctcggcgcctctgcgcgcgggaagatggcggaacaggctaccaagtccgtgctgtttgtgtgtctgggtaacatttgtcgatcacccattgcagaagcagttttcaggaaacttgtaaccgatcaaaacatctcagagaattggagggtagacagcgcggcaacttccggtgggtcattgacagcggtgctgtttctgactggaacgtgggccggtccccagacccaagagctgtgagctgcctaagaaatcatggcattcacacagcccataaagcaagacagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagcccagctctttgttcagttgacttactgtttcttaccttaaaaagtaattgtagatggaaatcagttgtgtttggcaggagaatcaataaaaatctttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggagggcaggtgccctctgctccccagtgctacctctctcttccctagggccttttgtggattgacagtagtcccctccgtaggagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaaaaaaaaaaaaaaaaaaaaaaaaaa

SEQ ID NO: 6 - DNA, 14188 bp, Homo sapiens
  Oligomeric Compound
caggtacccctctcctgcggccccgtcccctataggtaacctttacctcccgcggctccttcccctccaagcgacccgcagccccgccccctccaggtgactcccccccgccaacccccgccaccacacacacacaccccctcgccttccgcggccctactccctccaggtgaccccacccccgcagctcctcctcctccggacaacctgcagccccgcccctgcaggtgaacggcggcccagtccctgcaggtgacccgcggaccctccccgcccgtcctactaccgtcataggaccgcctccgcaggcgcactggagccgattgcgcaggcgtggctctcacacgcgctgccctgttggcgttggtgcgggactgcgcaggcgcgcggggcaagagggtggcagtgcgcctgcgccgcgtcggcgtgcggaacgccgcggtgtctcggcgcctctgcgcgcgggaagatggcggaacaggctaccaagtccgtgctgtttgtgtgtctgggtaagagggcgccgacttactcatgttctgacgtcctctggagagttggatcgggcttgtgcgctgtaggttgtgccgccggcctaggaaccatgagggggaggaggccagggactgggaggcctagggtgttctaggagtgtgccgcagcgcccctgttccccatccgccccgtgcacccgcccagcctgcccgctaaacctgggtcccctcgcgcctgccatatatccggggctcttggcatctgcagccacagcccctacaagccaaggtactttctttgcgactactagaatccctctcgttcccctccaatgggcccaggtaggcctggaggttgtcggactgatgtggtctcttaggggcctcctgagtcctatgacaacattgttttgtttgctagtttccttagcccatgcttagatagggatctgagttaaagggagatgaagttttacggtttcgggtttcttgattgtaaaagccaagcaaaggagacctggaggtccctatataactttggcccatgaaaaatgaaagtgtattttattaatggcttttcaggaactttccagcttaaattattttcactcctagaatttcaacacttagcttgccttacttcaaacataatttattttccgtttggtcttcagggatcttcctttggtgtagcactacaaggttatattcctcggtgcctccaccctgagaggaaggcctaaattgtcagtagtccggttacttaaaaccttctgtgagtttgcatttcctgcagtttctgtttatttttacagactcctcactttatctcctgtaccttttcctgctcacaccctcgggacactgccttccagaacctttcgttgttcggtagcacttcacatgcttactgagccttcatcgctgggtgcccattatgcattttgcctggaaagcctttttattttataaagtcaaacctaaatatttcctcctttgctttcccagatcatgagttgcttccttatcgacatcttttattacacaaattttactataatttgtgtaatatagtaaaagatgtaatacagaaaaggtgtgattccttatcgacatcttttattacacaaattttactataattatttgatagtctgttgaaatcccagctgagtttaattttgtcgatggcaataactgtggcttatttttcattttgttttcagctttaggacaatatatgataaacgctgactaggttttgtagaagttaacttctgtgaaagacgaattagaaggcttaccttgtttcacattggtagtgggcatcatttgatttttaagactcattgaaggctgaatgatgaaatcaataagtttattaaacagtagtttggttaaggatggttactaaataatattccattcccttgtatagttttggttggtattgtcgattatgctggcataatgcatcatggttgctgttaatggggaagtgtatccttgcatatacagtagtcccactttagctgtaggggatacattccaagactccccagtggatcccgaaactgtgggtagtaccaaacccaattgctgtcaattggaacatgtgtctgctcgtttcttacacccacaaatgtaataccgttgccaccttaactaagcatttaaaactgtggtcgcaacttttgcagtttgaggcaagacagcagacgatcaggaatttctttttccttgtttacattttcatggatagaagatttgttcgtactgtagatcttagtaacctcggtataagttttttttttctttccataagaagtcaaaaactttcacctttttacttaagggtagtgcttatggcttctctttgccgtatctgaattgctagcttcactactcttatgatttcgggccattattagtaaactaagggtgactcgaactcaggcactgtgatagcatcacaatggatctgataagctagagggcaataggtggatagacagcatggatatgctggacaaagacgtgtttcacttctggggcaggacggaatgggatggcatgagaatttatcctgctactcagaacagtgcacaatttaaaacttaggaattatttgtggaattcttcatttaatattttccaactgaagctgaccatggggtaaccatggaaagcaaaaccttggaaaagaggggactattgtatttcaatacaattcagtgattttttgttgttgttgagcatctactatatggcatgcattcctctaagtggtgtggtgaacacaacagacaaaaatcccagcctctctggagctcacgttctctcagtcaccagcaagtattaattgatgtgctcaatcggttgatgtgaagattaagaaaaatagagcagaaaaggggaatgggagtggagtgcagttttgagtgggatggtcagggaaggccccacctgatagatgacttttgagtaaagacttgaaggaggtgaggaagcaagcctaggatattttggagaagcatcctgaacccagaggaaagcaggtaaaagttttgaagggagaatactggccatggtcatgaaccgcagggaagctgcagtggctgggcctagtggccagatagtttttctcagtcaggattcctcatctatacctgaaaatttagcagataatttgagcgcctgttttctcagtgcttccattgtagatgtatagggagaagttaattccttatatacactggtcctttagggcaggagtctgcaaactctttgtttaaagaaccagattataattattttagattggcttcacatgacatacagaaagggtgtagcacaaagtagccatccatatgatgaacctggctgtgttctagtggatcttcatttgtggacatcagaattggatggaactctgtataattttcacatgtcacaaggtactctacttttagattttttccaactactcaacaatgtaaaaattattcttaccgctgtgggctgtacataaaggcagtggaccatgagccgattttcagacccctgttttaggtccagatcttgggaggccatgtgaagacttgggcttactctgaataaacattggagggttttgagcagaggtgggaagtgatcaaatttaagtttaaaaaagaagagtttcagctgttgttttgagatgaaactggaggtggagcatgcctttactggttgcattctgagggaatttgagcggtaaatggcattttacttggtagccatatagaggacagtaagagttgtatgcttttgaaatgtaaaaaactttgttcattcaaagcatttgtttggaccctattgagattgaagtgtagaaaagcatcagaggtttttcattggtgaagctaatgatggaaaataattgagctccttactacagcacaattatgaagggtgctttatgtttttgcaatagagacttattagtgaagatgatgcaggagccatgacccagccagcatggttggtggagatagcacatgctggtggagtcactttacttctgggctacacccatactgggtgctgggtctcttaccaggtatagcagacacagccccaccctcccagagcctatctgccttttatgagtctccatccttgaattgaagatgctgaagaagggtataaaatagtctgcctaatttggaaaagacaaacttgagactttacagattgaaacattttttaaaggcatttgaagatggaatgttctgtttttagaacttgtttgataggcgaaacctgaagtacatgcagtgtttcactgatgaccggggtgctccattttttcataacaaaatttggtggcatcaactttcgaaatcaacagggcaagctactgctagcctttaggacagttaataatgcaagttttcatattgttgtggggaaagtaagctgttattttacagttaaagataattatttgtaaacgtgtggggctagtctttattatatttttagatgctgagatactaggcatctgttgttctacagatccaaaataataattgtcttaatttggaatacaatgtttgctgttaaaaatattcccagggtatatggtgacaagtaaaagagttacacacccatgggaagcctttcaataattaaaatagtgctgatgacaatacctgtttagaaaactaaacaaaatttagaatgtcgaaaggaaaaacttcacaacacaaattagcatatagagggtattcaaggctaatttgttgtctttccctttatggtttgtaggtaagattttttactgctgtggaaactacagtctctgtgggaaaaaagtacgtaatatgcttaggtcttacaatgtagaaactgaaggctgtagtaaggcagtgagttggttttctgaactagggaattagctgtgttttacaacactagaagatcctcattgtatttgtttatcataaacagaagaccctaatagtgataaagagtaaactcttagttagtgctgcctattctcacagttcctctttttcgtatctgtttcctaaattcagctcctaatgtctggactcagtgtggtgctttggcattaaggagactggaaggcctcagagcagccccagaagcaaggtcgtagaaaccatcatttatcttccccagcgcagatcatagaaactagaactcctctcctgcaaagcaagccataaaacctagaaagatcactctctcccttttgccttctcttgaagacagcatttcagaggggccctgcctcattcctgggggaggaaatgctgtggccctgctgggtttccctcagtctgctaccattggatcatttccttttgtccagtgccatttctacacagctgtgcattggtcatctaagcatcaaaaccgtttccctgggtctttgggtcttcatttctgaaggctcttgttaacctgtcttttgttacaggagtatcttccatgacctttatgatgggtgaggaaagctgccacacctttccactctgacactgtcttgtatttgctccttcatacgagcctcttacaaggtctctccaattcagtcatgtgctcttctagtgtttccttagctgtgccggtaattttggtaacttgtaggaagggtctgtgttttaaacaactttctgtcttcaggttagggcacagtgcttggcacatagagttggcatacaaaatatgtttgttgaataagtcatattacccttttccataggactttctatttctcctcatagtggctacgcaaagttcaaatgttggcccagtgttttagggctcctgtataccagcccgtcagtttccagacactggctctctgtacctgaagcatcgcagcctcacctgcttagtgccacaatccctctgagttaaatgcctttcccttcctctgtccattaagatcctgatcatccttttaatatcatctctaatctttcttttttctggtaagaaattttcttatatcttgtttttgctttgtagcatcgttctttttctgtgctgctgtagtgtattgtttatcagcattcattttcccacttttattataataaatataataaataaagtggagatagagttcagctgaatggccagtgattcaacccattgtgtctccctccgaaaaccccaataaaacctcagagcactatggctctgaggagcttcctgggttggcagtgctctttgaatattgtctcatgttgatgacaggagagtaaggtgtcccagaggacagtggagctacctgttagggaacctcccagaatgtgccctataggtcacttgctttggctgttttgtttgtttgtttgtttttgtttttgttgctgttactataatgaaattgtaatggtaagtgttctgctttcctgagtttggggagtaattctagggaattatcaaacctgagggagccctggggaacctggatttctcacttgctggtctaaagtaaggatggccctgatgtcgacatcgtggtctggagagtggtgtccctgaccttgggctgctaatgcctggatctgtatccctgggctcagggctttgcatgtgatcgctcagtaaatgttggtgttgaatcgctgtttatgcttaaatgcatgtttgtgtgtttgtgtattgttttgtgaaacagtagtcatccttatgtgtgatatacttgaaaatacatacagttgacccttcagccacaggggtttgaactttgaaggatttattcgaggaatttttttaatcaagcacagatggaaaataccgtgttgtctgaatgagaaacccatctgtatggagggctgacttttcctatacttgggctccacattctcaggggccaactataggacctgagtatgcacgttttggtatactgggtaggggtgggagggtgtcctgaaccagctcctgagtctactgagggacagaccactgtattttatttttattctgttgtatttcattttaaaataagctggttgcaacccacttcattttcgtgattcactcctggatcattagtagcaactctttaaacaataatttttgttgaaattgtttttctgatatctttgtcatctgtacttggagtacttggctggttggtgtcaaatcactgaacttgagcctgataggctgttctccagcagggtctaccctcactggttggacaaaggcgtcaaaggatagtgttggcctttgtttttcctgagggatagcacagcccccgtgtgtcaggtgtgtaaagaaggggagctggcatgtgcccttccatccaacctaaccctgtttccccacccctccctttttttaaaggtaacatttgtcgatcacccattgcagaagcagttttcaggaaacttgtaaccgatcaaaacatctcagagaatgtaagtaccattcattatcttaaagaggccaacctgaactcctctgggcaggaaattgcaaaaaaaaaaaaaaaattccatgtttcttcccctgcagtggagggtagacagcgcggcaacttccgggtatgagatagggaacccccctgactaccgagggcagagctgcatgaagaggcacggcattcccatgagccacgttgcccggcaggtaccgtccttggacttgaagttgtgtgttttgtgtttcagtgggtcattgacagcggtgctgtttctgactggaacgtgggccggtccccagacccaagagctgtgagctgcctaagaaatcatggcattcacacagcccataaagcaagacaggtagacaagctcttgttcaatttctaatatatagagtccagtaacttgagaagtagcgaaaggattaaccagacttgtatattaatgaatgtgtttatttagggtgagcttaaccagctatggtgtgtccattttgtttcacttctggttgcacggtgttgaaagacttgcctgactttggaatttacttattaaaatgcacataaaagctaggtaatttataatgagagagcctgactgtgagctggggctgagcggtgctctgtcttctgttccttcctgcataatttttattaaacatttaggccatagtaatcatcctgctgatattgcaagtttgttgctagaatgaggttatataatatatacaaaaacattttttcaactgtaaagtgccttagtaatatagggtaataccagcaacattatggatatataattatagtctattgggccacacttaagtttggagtctaataaagtcacaatcaaattctgcaatttcaattgaagataaccttgtctttatattatgaattagaagctaaagttgatttttctaagagttctttatttaaatgaagtactctgggactgaccttttcggaaatggaatcttcattggtcaggtgattcaacatttttatacaatttatccatcctcatctcttcaggatttgcataccttgccagtttctactggccattgttgaaaatacatttatttggagaagtccaaagccaaggggctcatggggctgtgaagtccttcttgctgcatcgtcctgtggtagaaggtggaggagtcaagagagtgccccagagtgagtgagagcgagaactagaaaaacgggaagagggaaacagaggagagagagagagaggacccatcagtgtcaggagcccactcccaagatagtggcattaataaagatcctgcgtctcactattgttgcattggggatgaagtttccaacaaaggaactttgggggacacatccaaaccataacataggatttaaataattttacagagttcaagagttctgctactgaaccgtttgagatccctgttctgaggtctcatcactttccagttttagcaggaagagaagtggcaagtggcaggagtctgcagattggggcctgcacctttttttgaggcaccttttttatgaacagtgtttgttggaacacagccagactcactcattcacctgtagttcgcggctgcttttgtgctagagcagcacagccagagcggcctggtggccacaaagcccgactgtctctgcagcactggttttttatatggcgtttctagttgtttttagtagtaggggtgatttgccacaagctgtttcatttatagctgcaagtggaaatcctttattgtgcatttgttacataagttatagctgtttctttctcaattattgtaacatctaaaaattatgtaacagttactaagtctttttttatgaaaaatttacaattagtccagaatttaaaacaagtttagtattaaatcctagggaatttctcacataactaatttggagaaataatataatactgttcaggcatggtggctcacacttataatcctaataacactttgggaggctcaacattgcttgagcctaggaggtcaagaccagcctgagcaacatagtgagacccatctctaccaaaaaaattttaaaaattagctgggtgctatgacatatgcctgtagtcccagctgtttgggaggctgaggtaggaggttggcttgaactcaagaagttgaggctgcagtgagctatgatcacaccactgtaccccagtgttggtgacagagtgagaccttttctctaaaaacaaaaagaaaaaattcagtattgtacttattctcaattttgaaataagtcaatgtcatgggagtgttgacgaaatacagtcttttctagctactggaagtgcatactaaaagccaaagtttctcattttttagaagaacaggtatacactttccacctttgttccgcagtacgtatgtttatgtttcttattctgccattatttatagtagtcgaaattcacaaaataatcttttcatttgtattttaagtaggtgtgtttatagattatatcagaaaacaatcataagctttcaggttacaatcagtagaaatactgagtggcattctcttctgtgtgaggtttaaattatggatagtcaatcaaaataaaggaggactgtcagttatcactcctgtgtttctttcttagtgaccacctctgaccacccaacttaatacttttactcctccctgccccaaagctgtatgtatctcctctctgctttatttcctccagggcatctgtgcctcctgtgtgttgcctgcccccatgtggaagggcagctccatgggacgcggctctgtccacttagcttggtgtttgttactggggcccaggatgttcagtgcttgtggaatgaaaaatagagtagaataatgaaggggattaaatatgtgacattttagtatgttgactgtattatacactgctactaaggaattgaagccgatgtataaacattgtgtctacacattatactattttatgattattgtatggaactttataatacaaaatttctttgcagtacaattttgagaaataggttaactctattttaacttaaaagtaccctaaaaattatttaattgtttattagtgagtaacaggctcaaatacagcagttttttttttcttttagtattgctttgcatcctctaggcttgaatggtataaacactgtgttttgacttcttattcaattttagattaccaaagaagattttgccacatttgattatatactatgtatggatgaaagcaatctgaggtaatcctgtttttgaagaatatttctgttcaactctcagttcagcagtgggccaagtaatttgttgtccagatttactttttctattttaaaggttttaatagtcagtgatggtcaccatatgtagaattattttatttagaacagcaaaaaacttagtaatctagaattgtctcttaggtatttacaaggaaatacaccttagaaaagggaaagtctagttgttaatagcatgatttaattggaaaactagcttggctgttatagttttgtatcaagcatattgttctgcatgagaacgattttgatatttttgctgtaatgattcctgggcagggcagtgttttattgattctcaggtcaaagctctgcaaccataggttcctattattatcccatcttgtaaaagaaggaagtgagaaacggtaatgttaggttatgtccccgagttcacacagctagcatgtggtggatgtcatgggatttaagcctaggctcagtgaggaagggccctgctgtatgcacccctgcctcagtcaccaccctcctgacctgccccagtcccatgttcaaggcgtcatcgggaacggatcataatttaatgtcactccaggattctgacttgcatcactacttattcataaatatctcatgtttttgatgaagataaactatttgtgcttttaattacttttgtacaaatgaacagatagggatgcaccagtttcaattattttttattaattaggaccttcattaaaatgcatgtattttttaatttttatttttcagtccttattctgttctcattgtttttgctgacaccatagcccataggcatcgtaaaaacacctttttaggagaccccatttgctttgactggcactgagcaccgtgtttctttgcgtgtggggcaagtggagttcctgcagcctcacatttgcacgtgctgttcctctgcctggaagctcctccttgcttcttgggtcattcggctcctatttgcccctcgtgggtcagcttaaattcatttcttgatagaggtattctgctgccctgtcattaggccagattgtgcattagacttaggcctgcctgcagttctcattcctgcacctaatcctgagctgtaaaacttcgtgcatgggagctcttggctgttccgttcccacataaagcacgtacctgactcatcaactcatctgtcgcaggtgctcagtcgaaatttgttaaataaatggaggcgcttagttaagtttgcctttttcttttgtagcaatgtgaaagctaagggtggaagtctagagtgaagctgagttttcagcttgggcagaggcttaaggaataaaagatggaagagtaattaaggagtgggtcgtgctgtgggaagtgctgtgtcaagacacctgaggatggaattggtagccctctgttttgggactggttcagatggtgttttgggttcctttctatgacctttatctctgaaacttgattgtcttaaaatgtattttgtgggagaaaatataattattgtatattttgtgtaacagaatcagtgagaataagctgctgtcgcaaactgtcttgcctagagagagggcagtggcatacagctgcagaaggggccacacgggcctgctgagtgttccgtttcatttcaaattaggtcattctgtcttgattttgatatggatgtttcagaacaccctagcagatgtccctgtttaacttgaaaccatagatcagaaaactaagttcatatttcaattttacagagatttgaatagaaaaagtaatcaagttaaaacctgcaaagctaaaattgaactacttgggagctatgatccacaaaaacaacttattattgaagatccctattatgtaagtacagttcacgttttagggctaatatgaagacccaacacatttgtatcctgccatattaaataacagatgagattgtgttaaggatgtttttgttatgcaggttttgccattttcttctttttcctgtccatttaggggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagcccagctctttgttcagttgacttactgtttcttaccttaaaaagtaattgtagatggaaatcagttgtgtttggcaggagaatcaataaaaatctttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggagggcaggtgccctctgctccccagtgctacctctctcttccctagggccttttgtggattgacagtagtcccctccgtaggagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaatctggtgtctgcctgtttttttattgacaggtaaggaagcatttgaaaacattgtattgtcctttttacatgatctgtgatgttaggtgtgcatattttatcttcaggataaggaagtaaaatgatgaaaataagcacattgctgccccatgtcatcatgtctagtttctgaaggaagtttgaggttctacagctggaactgtccttgcccagtaccatcctctggacatgttgcctctacgctgtgccctcctgtgcagtgatgcacagctgcctggccaggacatctgcactcctgacctcagtatcatgagggtcccaggcagtgccatggggaggagacacgtttgatccttgactccctagtatctggaggataagctgttgccagatgaaagtg

SEQ ID NO: 7 - DNA, 10191 bp, Homo sapiens
  misc_feature 289: n = A,T,C or G
aagctttctctgctccctcggaacaggaaagccctttatttattcctgcccttcctcttctccaactgaagagtgtgatcttggtacagagcagttccacacatgccacactggtctgatcttttagagtaaaaaatgggatcttctcagagaaaatttttttaaaaacttcatgtgcttaggtctcagagcagagaattttgctcttatcatgacgccactcactggcaaacaagctggcgcacagggacttgtgaggctctcagcagagtcagggtagcagttattnccttgactgctcaacttctatgtttgtgttgacatgccagatccttatttctgcggaatgagaaatctcttgagaataaagttttcctaaacagaacaaaagtatcaaatcacttttgatgagcaaacattattttaaacttttttttaacttacaaaagatgaggggtaacatttactagttttacagaaattttctttaaacagaacattagtgaaccaaataaccaattctcctcaaacatgatagactaaacatcagtcgctgcacaagtattttaataacaggccagcccacgggagtaaacagcaccttagcctcgagacctgttctctcaactgtgtgtacagattttctaagtcgttatgaattcagatattccctacatctcaataaaaacacctaggagaacaagaaatcaggacatgcatctcctgtaagcagaagagaaaccgagcacctgctctcttgcgggggggcagcttcgtgtcactttccaactcagcctccacaggagcatgggcatattggttcaaatgaagtgtaaaggacacatctctgtccacagtgtaaatcctatttccttaaggtggtcaaccgattgagaccagttccacacttccttaaagaaggaaaaactcagcagccagcgccaaggcccagcgatcagagaggacccgtgctcgccgcgaccccgctcgccctgtggccccgcgaccccggcgcaggtgttggggggacggcccttcccttccggtgtgaggggctggcttctcttcaggttgggggctggcaaactcgaacctcgccggcgcctagcaaaggcttcctccccggcgccttcggcccgcgtttccaacgcacattccaagcgcgcgcattcgacccaaacttcagagacgcttctgtggaacggaacggatggtcgctgacacctcgccgttcaaatatcaggaagttaaaaataacacgaccatcgtcatcattccaaacttcggaatagcttttctaaaaatggaaaagacggtggctaaacttcaatcaaaatcgctgtggtcaagaaagtcctggaagctgctctatcttaactttgggcggtttgcggttaacagacgcgcgacctagaaaccccagaggcatcgccggttttcccgtcctcctcctcccaccaccgcccagctcttgagaggggagggtgctgccggacaggtgggtccccgggtactcactgctgcccgcccggccgcggcgccccgtcccgaggctgcccaggaagaggaaggcgcgctgccccgccccgcggacaaggaggccccagcgagggcgtacctgcggcaggtgacgaaggaggcggcgcaaaacacccgccgtgtacgtttcgcgggacaaaaaccacgcgcccgccgggccgcgctcaggcttcgcctcaggacttcggaaccgccccgtcctcaagatcgaaaagcccagagccgcgcgctcaagcacggtgttgggggtggggtctcaggagcgcccagcaaggcgcccctgtcggcgtggacccgcgggctcaaaggcagttccccggtgacccgcccagcccctctatgcgaactcgaacgacagcaccacagcccgccagcgtcgagactcgcgctgtgccccaacccaggtgggcgccgcggagccgcgagcctgagcccgccctgcaggtgacccgcggcccttcctcctccaggtaccccttctcctgcggccccgtcccctataggtaacctttacctcccgcggctccttcccctccaagcgacccgcagcgcccgggccctccaggtgactgcgcccccgccaacccccgccccaccacacacacacaccccctcgccttcttccgccctactccctccaggtgaccccacccccgcagctcctcctcctccggacaacctgcagccccgcccctgcaggtgaacggcggcccagtccctgcaggtgacccgcggaccctccccgcccgtcctactaccgtcataggacgcctccgcaggcgcactggagccgattgcgcaggagccgattgcgcaggcgtggctctcacacgcgctgccctgttggcgttggtgcgggactgcgcaggcgcgcgggggcaagactggcagtgcgcctgcgcgcgtcggcgtgcggaacgccgcggtgtctcggcgcctctgcgcgcgggaagatggcggaacaggctaccaagtccgtgctgtttgtgtgtctgggtaagagggcgccgacttactcatagttctgacgtcctctggagagttggatcgggcttgtgcgctgtaggttgtgccgccgcgctaggaaccatgagggggaggaggccagggactggaggcctagggtgttctaggagtgtgccgcagcgcccctgttccccatccgccccgtgcacccgcccagcctgcccgctaaacctgggtcccctcgcgcctgccatatatccgggcctcttggcatctgcagccacagcccctacaagccaaggtactttctttgcgactactagaatccctctcgttcccctccaatgggcccaggtaggcctggaggttgtcggactgatgtggtctcttaggggcctcctgagtcctatgacaacattgttttgtttgctagtttccttagcccatgcttagatagggatctgagttaaagggagatgaaagttttaccgtttcgcggtttcttgcattgtaaaagccaagcaaaggagacctggaggtcctatataactttgcccatgaaaatgaaagtgtatttatatgctttcaggaacttccagcttaaattattttcactcctagaatttcaacacttagcttgccttacttcaaacataatttattttcgtttggtcttcagggatcttcctttggtgtagcactacaaggttatattcctcgtgcctccaccctgagaggaaggcctaaattgtcagtagtccggttacttaaaaccttctgtgagtttgcatttcctgcagtttctgtttatttttacagactcctactttatctcctgtaccttttcctgctcacaccctcgggacactgccttccagaacctttcgttgttcggtagcacttcacatgcttactgagccttcatcgctgggtgcccattatgcattttgcctggaaagcctttttattttataaagtcaaacctaaatatttcctcctttgctttcccagatcatgagttgcttcttatcgcatcttttattacacaaatttactataattatttgatagtctgttgaaatcccagctgagtttaattttgtcgatggcaataactgtggcttatttttcattttgttttcagctttaggacaatatatgataaacgctgactaggttttgtagaagttaacttctgtgaaagacgaattagaaggcttaccttgtttcacattggtagtgggcatcatttgatttttaagactcattgaaggctgaatgatgaaatcaataagtttattaaacagtagtttggttaaggatggttactaaataatattccattcccttgtatagttttggttggtattgtcgattatgctggcataattgcatcatggttgctgttaatggggaagtgtatccttgcatatacagtagtcccactttagctgtaggggatacattccaagactccccagtggatcccgaaactgtgggtagtaccaaacccaattcctgtcaattggaacatgtgtctgctcgtttcttacacccacaaatgtaataccgttgccaccttaactaacatttaaaactgtggtcgcaacttttgcagtttgaggcaagacagcagacgcatcaggaatttctttcctgtttacattttcatggatagaagatttgttcgtactgtagatcttagtaaccttgtttacattttcatggatagaagatttgttcgtactgtagatcttagtaacctcggtaataagttttttttttctttccataagaagtcgaaaaagctttcacctttttacttaagggtagtagcttatggcttctctttgccgtatctgaattgctagcttcactactcttatgatttcgggccattattagtaaactaagggtgactcgaactcaggcactgtgatagcatcacaatggatctgataagctagagggcaataggtggatagacagcatggatatgctggacaaagacgtgtttcacttctggggcaggacggaatgggatggcatgagaatttatcctgctactcagaacagtgcacaatttaaaacttaggaattatttgtggaattcttcatttaatattttccaactgaagctgacatggggtaacgatggaagcaaaaccttggaaaagaggggactattgtatttcaatacaattcagtgattttttgttgttgttgagcatctactatatggcatgcattcctctaagtggtgtggtgaaccaaacagacaaaaatcccagcctctctggagctcacgttctctcagtcaccagcaagtattaattgatgtgctcaatcggttgatgtgaagattaagaaaatagagcagaaaggggaatgggagtggagtgcagttttgagtgggatggtcagggaaggccccacctgatagatgacttttgagtaaagacttgaaggaggtgaggaagaagcctaggatattttggagaagcatcctgaacccagaggaaagcaggtaaaagttttgaagggagaatactggccatggtcatgaaccgcaggaagctgcagtggctgggctagtgcagcgatagtttttctcagtcaggattcctcatctatacctgaaaatttagcagataatttgagcgcctgttttctcagtgcttccattgtagatgtatagggagaagttaattccttatatacactggtcctttagggcaggagtctgcaaactctttgtttaaagaaccagattataattattttagattggcttcacatgacatacagaaagggtgtagcacaaagtagccatccatatgatgaacctggctgtgttctagtggatcttcatttgtggacatcagaattggatggaactctgtataattttcacatgtcacaaggtactctacttttagattttttccaactactcaacaatgtaaaaattattcttaccgctgtgggctgtacataaaggcagtggtccatgagccgattttcagacccctgttttaggtccagatcttgggaggccatgtgaagacttgggcttactctgaataaacattggagggttttgagcagaggtgggaagtgatcaaatttaagtttaaaaagaagagtttcagctgttgttttgagatgaaactggaggtggagcatgcctttactggttgcattctgagggaatttgagcggtaaatggcattttacttggtagccatatagaggacagtaagagttgtatgcttttgaaatgtaaaaaactttgttcattcaaagcatttgtttggaccctggtgagattgaagtgtagaaaagcatcagaggtttttcattggtgaagctaatgatggaaatataattgagctccttactacagcacaattatgaagggtgctttatgtttttgcaatagagacttattagtgaagatgatgcaggagccatgacccagccagcatggttggtggagatagcacatgctggtggagtcactttacttctggagctcacatactgggtgctgggtctcttaccaggtatagcagacacagccccaccctcccagagcctatctgccttttatgagtctccatccttgaattgaagatgctgaagaagggtataaaatagtctgcctaatttggaaaagacaaacttgagactttacagattgaaacattttttaaaggcatttgaagatggaatgttctgtttttagaacttgtttgataggcgaaacctgaagtacatgcagtgtttcactgatgaccggggtgctccattttttcataacaaaatttggtgggcatcaactttcgaaatcaacagggcaagctagccatttagacagttaataatgcaagttttcatattgttgtggggaaagtaagctgttattttacagttaaagataattatttgtaaagctgtggggctagtctttattatatttttagatgctgagatactaggcatctgttgttctacagatccaaaataataattgtcttaatttggaatacaatgtttgctgttaaaaatattcccagggtatatggtgacaagtaaaagagttacacacccatggaagcctttcaataattaaaatagtgcctgatgacaatacctgtttagaaactaaacaaaatttagaatgtcgaaagaaaacttcacaacacaaattagcatatagaggtattcaagctatttgtgtctttccctttatcggttgtaggtagatttttactgctgtgaaactacagtctctgtgggaaaaaagtacgtaatatgcttaggtcttacaatgtagaaactgaaggctgtagtaaggcagtgagttggttttctgaactagggaattagctgttttacaacactagaagatccttcattgtatttgtttatcataaacagaagaccctaatagtgataaagagctaaactcttagttagtgctgcctattctcacagttcctctttttcgtatctgtttcctaaattcagctcctaatgtctggactcagtgtggtgctttggcattaaggagactggaaggcctcagagcagccccagaagcaaggtcgtagaaaccatcatttatcttccccagcgcagatcatagaaactagaactcctctcctgcaaagcaagccataaaacctagaaagatcactctctcccttttgccttctcttgaagacagcatttcagaggggccctgctcattcctgggggaggaaatgctgtggtcctgctgggtttccctcagtctgctaccattggatcatttccttttgtccagtgccatttctacacagctgtgcattggtcatctaagcatcaaaaccgtttccctgggtctttgggtcttcatttctgaaggctcttgttaacctgtcttttgttacaggagtatcttccatgacctttatgatgggtgaggaaagctgccacacctttccactctgacactgtcttgtatttgctccttcatacgagcctcttacaaggtctctccaattcagtcatgtgctcttctagtgtttccttagctgtgccggtaattttggtaacttgtaggaagggtctgtgttttaaacaactttctgtcttcaggttagggcacagcttggcatagagttggcatacaaaatatgtttgttgaataagtcatattatcccttttccataggactttctatttctcctcatagtcggctacgcaaagttcaaatgctggcccagtgtttagggctcctgtataccagcccgtcagtttcagacactggctctctgtacctgaagcatcgcagcctcaacctgcttagtgccacaatccctctgagttaaatgcctttcccttcctctgtccattaagatcctgatcatccttttaatatcatctctaatctttcttttttctggtaagaaattttcttatatcttgtttttgctttgtagcatcgttctttttctgtgctgctgtagtgtattgtttatcagcattcattttcccacttttattataataaatataataaataaagtggagatagagttcagctgaatggccaggtgattcaacccattgtgtctccctccgaaaaacccccaatacacacgacgcactatgctgctgaaggagcttcctgggttggcagtgctctttgaatattgtctcatgttgatgacaggagagtaaggtgcccagagggcacagtggagctacctgttaggaacctcccagaaatgccctataggtcacttgctttggggctgttttgtttgtttgtttgtttgtttttgtttttgttgctgttactataatgaaattgtaatggtaagtgttctgctttcctgagtttggggagtaattctaggggattatcaaacctgagggagccctggggaacctggatttctcacttgctggtctaaagtaaggatggccctgatgtcgacatcgtggtctggagagtggtgtccctgaccttgggctgctaatgcctggatctgtatccctgggctcaggctttgcatgtgatcgctcagtaaatgttggtgttgaatcgctgtttatgcttaaatgcatgtttgtgtgtttgtgtattgttttgtgaaacagtagtcatccttatgtgtgatatacttgaaaatacatacagttgacccttcagccacaggggtttgaactttgaaggatttattcgaggaatttttttcaatcaagcacagatggaaataccgtgttgtctgaatgcagaaacccatctgtatgagggcctgactttcctatactgggctcacattctcagggccaactatagaccctgagtatgcagcgttttggtatactgggtagggtgggagggtgtcctgaaccagctgcctgagtctactgagggacagaccactgtattttatttttattctgttgtatttcattttaaaataagctggttgcaacccacttcattttcgtgattcactcctggatcattagtagcaactctttaaacaataattttgttgaaattgtttctgatatctttgtcatctgtacttggagtacttggctggttggtgtcaaatcactgaacttgagcctgataggctgttctccagcagggtctaccctcactggttggacaaaggcgtcaaaggatagtgttggcctttgtttttcctgagggatagcacagcccccgtgtgtcaggtgtgtaaagaaggggagctggcatgtgcccttccatccaacctaaccctgtttccccacccctccctttttttaaaggtaacatttgtcgatcacccattgcagaagcagttttcaggaaacttgtaaccgatcaaaacatctcagagaatgtaagtaccattcaggatcttaaagaggccaacctgaactcctctgggcaggaaattgcaaaaaaaaaaaaaaattccatgtttcttccctgcagtggagggtagacagcgcggcaacttccgggtatgagatagggaacccccctgactaccgagggcagagctgcatgaagaggcacggcattcccatgagccacgttgcccggcaggtaccgtccttggacttgaagttgtgtgttttgtgtttcagtgggtcattgacagcggtgctgtttctgactggaacgtgggccggtccccagacccaagagctgtgagctgcctaaagaaatcatggcattcacacagcccataaagcaagacaggtagacaagctcttgttcaatttctaatatatagagtccagtaacttgagaagtagcgaaaggattaaccagacttgtatattaatgaatgtgtttatttagggtgagcttaccagctatgtgtgtccattttgtttcacttctggttgcacggtgttgaaagrcttgcctgactttggaatttacttattaaaatgcacataaaagctaggtaatttataatgagagagcctgactgtgcgctggggctgagcggtgctctgtcttctgttccttcctgcataatttttattaaacatttaggccatagtaatcatcctgctgatattgcaagtttgttgctagaatgaggttatataatatatacaaaaacattttttcaactgtaaagtgccttagtaatatagggtaataccagcaacat

SEQ ID NO: 8 - DNA, 769 bp, Homo sapiens
  misc_feature 43: n = A,T,C or G
tatggatatataattatagtctattgggccacacttaagtttnttgagaatagtactcattactacagtaccctaaattattatgttattagtgagtaacaggctcaatacagcagttttttttttcttttagtatgctttgcatcctctaggcttgaatggtataaacactgtgtttcgacctcttattcaattttagattaccaaagaagattttgccacatttgattatatactatgtatggatgaaagcaatctgaggtaatcctgtttttgaagaatatttctgttcaactctcagttcagcagtgggccaagtatttgttgtccagatttactttttctattttaaaggttttaatagtcagtgatggtcaccatatgtagaattattttatttagaacagcaaaaacttagtaatctagaattgtctcttaggtatttacaaggaaatacaccttagaaaagggaaagtctagttgttaatagcatgatttaattggaaaactagcttggctgttatagttttgtatcaaagcatattgtttctgcatgagaaacgattttgatatttttgctgtaatgattcctgggcagggcagtgttttattgattctcaggtcaaacgtctgcaccataggttcctattattatcccatcttgtaaaagaaggaagtgagaaacggtaatgttaggttatgtccccgagttcacacagctagccatgtgtggatgtcatgggatttaagccctaggacttcagtagga

SEQ ID NO: 9 - DNA, 2053 bp, Homo sapiens
ctttatctgaaacttgattgtcttaaatgtatttgtggagaaatataattattgtatattttgtgtaacagaatcagtgagaataagctggtcgcaaactgtcttgcctagagagagggcagtggcatacagctgcagaagcgcgacacgggcctgctgagtgttccgtttcatttcaaattaggtcattctgtcttgattttgatatggatgtttcagaagaccctagcagatgtccctgtttaacttgaaaccatagatcagaaaactaagttcatatttcaattttacagagatttgaatagaaaaagtaatcaagttaaaacctgcaaagctaaaattgaactacttgggagctatgatccacaaaaacaacttattattgaagatccctattatgtaagtacagttcacgttttagggctaatatgaagacccaacacatttgtatcctgccatattaaataacagatgagattgtgttaaggatgtttttgttatgcaggttttgccattttcttctttttcctgtccatttaggggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtgtgtaatcacgttccagggcccaaagccagctctttgttcagttgacttactgtttcttaccttaaaaagtaattgtagatggaaatcagttgtgtttggcaggagaatcaataaaaatgtttgattcagacagcttatggggtattttaagcattcttagactagttgaacatctcactttgccccagttacaaaaatagtagaacaagcaacataaaacaatgaaggaaaacctcacttgaaggcccaggtcaacatctaagcctgttgagacttagataatcgagtctacctcttcagtaggtttgtgtggatggcctggaggcaggtgcttgctccccagtgctacctctctcttccctagggccttttgtggattgacagtagtcccctccgtagagctcacagtctagattagaagtgttttaatttctacacacccatagtgcacacttgtatattgaaaagatagggaagagagaaacatttatggaatcagtcgttggcaccttcaatacttcatgatttttgtcgagtttacttcatgaggaggtcagcccattggctcccatctgaaccactttgcctctgaaacttaattacatccagaaagaaggacacttgtatgctagtctatggtcagttgaggaatatgactgtttttatatgcacatgtaacccaaatgtccaatataaattggcttattttttaaaataattttaaaagttgggaaaagtgttattatttggcatgcttaaatattgaataagtattcttcatcagcatttaataaatgtataggcagatgtaaggtaatttctgtgtattttgagataatgtcaaaatcatgaatatttcaaaataaactggggagttataaaaatacaactagagatataaatctgtgtctgtcctgttgttgattgacaggtaggaagcatttgcaaaacattgtattgtcctttttacatgatctgtgatgttaggcgtgcgtgccatattttagtcttcaggggataaggaagtaagacatgatgaaaagtcagcacgactgctgccccatgtcatcatgtctagtttctgaaggaagtttgaggttctacagctggaactgtccttgcccagtaccatcctctggacatgttgcctctacgctgtgccctcctgtgcagtgatcgactgcctggccaggacatctgcactcctgacctcagtatcatgagggtcccaggcagtgcatgggaggagacacgtttgatcctttgactccctagtatctttgaggataagcttgtttgcccagatgaaaagtgagtagtttcccactgtacccatgtggaagtgcccagggctgtcttcatag

SEQ ID NO: 10 - DNA, 577 bp, Homo sapiens
atggcggaacaggctaccaagtccgtgctgtttgtgtgtctgggtaacatttgtcgatcacccattgcagaagcagttttcaggaaacttgtaaccgatcaaaacatctcagagaattggagggtagacagcgcggcaacttccggtgggtcattgatagcggtgctgtttctgactggaacgtgggccggtccccagaccaagagctgtggagctgcctaagaaatcatggcattcacacagcccataaagcaagacagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtg

SEQ ID NO: 11 - DNA, 1004 bp, Mus musculus
cagcaatgccttaggtgctgcaaggccttcctggagaagacttactagctggtcctaagccccaccattgagcagctcactcctcagtgctgtgcccaagggtggtggcagtccttagccccataccccacctctcttttcagctgacttactgtatatctttaaaataattgtaggtgggaattaggcatatgttcagaaggataaaagcatttgagtcagacagtttgaggtgtggctaagcattcttagactaactaaacctctgaccttgcggtgattacaaaacagtggaacaagcaaatatggaacaaaaaaaaaaaaaaaaaaaaaaaaaaaaaaaaaaaaaaaaaaaaaaaaaaaaaaaaaaaaaaacaaaaaaaaggggggggggccccaaaacccccgagggaggagccgccccagataaatctcaccccaccccccattttttttgaaaaaacggggcccccctaagaggaggagcgagcatacataaaaccgcagacggagcgagcgcgccggacacaacacaacacgacgcgggaggcgacaaccaacgtcagtcctagccggactacatgtggcaggagcaccccacatgcggggccggggaaacaactggcggcaacccctcccacagaaaataagggcccgagcagaatacacacacgcacgcggccgtcgcggtcgtaccacccccgccgcatttacgggggcgcgaaaacatttggccccccgacggagtaacacgaacaccttcgggccgcgccgtgaacaaacaaccccccacccaccccccaccacgcgacccccccaccaccagcgacgacgcgccaacgccacacacgaccagccagaccgcacgcaacgaccccacccgccactaccacccaacccacgacgcgacccccacccaccgcacgcccgacacgacaccacccagccaccccacccgccgcccaccccccccacccacccacacctcaacagagcaccacaac

SEQ ID NO: 12 - DNA, 738 bp, Mus musculus
aggtcagttatagactaagcaatctccatcatttagcatatttctggtgcacaggaagcaacgtgaggatcactcttcatctactcccattacaaagtctaccattataattttgatatgcttgcattctctattaattgaaataatatacaactcatgtgtatatgtcattacaaagagttttgcttcatggaagcatgaaagtatcacatatttatggcagagataagaattagagaaattgagggtctgcaccgaaacatggcagaggttgggtccaagtcagtgctgttcgtgtgtctcggtaacatttgccggtcacccattgcagaagcagtattcaggaaactggtaactgatgaaaaggtttcagataattgggccattgacagcagcgccgtttccgactggaacgtgggccggcccccagacccaagagctgtcagctgcctaagaaatcatggcattagcacagcccataaggcaagacagattacaaaagaagactttgccacattcgattatatactatgtatggatgaaagcaatctgagagatctcaatagaaaaagtaatcaagttaaaaactgcaaagctaaaattgagctacttgggagctatgatccacagaaacagctcatcattgaagatccctattatggcaatgactctgacttcgaggtggtgtaccagcaatgccttaggtgctgcaaggccttcctggagaagacttactag

SEQ ID NO: 13 - DNA, 19031 bp, Mus musculus
accgtggcccggcctggggtcggtggtccaaaggggtaacttagcttggaagggcgggtagctccgatcggaggagccaccctaggaggctctgcaagccgctatccttgagttcattggctgccctggcacatcctaatcctccctgctcaacaccgtgccacccaagctgctctgggggtcgccccgcgttcaccccgccctcactccttagacgtgcgacctgggcagagctcagagtgaacgctagcccgggctgccaacccgaacgcctaagccccgccctctaggcgacttctggccccgcctctggccgcgtacgaacgtggggcggcggactgcgcatgtccacggtcagaaccctggcagtgcgcatgcgcctcgtcccgacgcggtttctgggcgccagggtctgcaccgaaacatggcagaggttgggtccaagtcagtgctgttcgtgtgtctcggtaaggactcgccgacttaagggtctttttctttgggaccctgaagtgctctccctgttaggcttccgaacaagagcgctgggcggaggaagcagatcagaaggccttggctgggcccgcgtctgagcacatactcgtttgtctgcctttttcatatgcatcccccatcatctggtactcctctgagcccgagatctgtgtaatttttgtcccagattatgcccttccaaagcccatgtcacccttccgagtgcccttcaaccacaaatctttgtgcgagttctctctctcaagcccgtagacccctctcacttgctcctcctcttttaagtctgagagtcgcccatcatctgctttggcagttacttccaaatctctgtgctgaaaaatatccaaccaaaacaactttagggagaacagatttgttttgactcagtttcagaggttcaggccccttttgaatggttgtagattctgggcttgtggtgaggcaaggcatcatggtccttcaaggtggccagagcatgagtcagggactatttgctcctgtggacgggaatcacagaaggggagagtagagagcaagacaagcgttagccccaagagcacgcatcgggatgtattttcttctgctcagccacacttgtatctttcattacctccagtcatttcctcatattttgaatctactgggttaaaccattgactaggtcagtgcccttatgatcacatcctcgggggataagccatcaccgacgtcctgagagttgtctttttctaatcagttaaggtctcgatgacgattaatcatcatccctgttattccctcatgctcttgtccgtcacctaacttgcttttcttcctttgaaccctctcacctgctaactggcccaggtggcttcatatctgtagtgtgactctctaagctggccccttctattagaacagctggagttcttgccggaaacccccacccccacccctgttccccattgtgccaacgtgagcctaggttacaggaagaccattctgtgacaggtttgtttgatgtcatccttgggtcacagttgctatggttctcagccagtctcttttctttaggtctctctgaaaccttggttttcagtccctaggacaattactcatgccatgtgaaaaacagtttgtttttaaatgaataatcttttaagtaccttctaacacagaatttcaaccttgattctcctttacataatatgcaattctttcttttttcagagttcttgcctccttgactccaccctgaggggcaagggttaaattgtcaacagtccagctatttgtcttttgtcctctgcttttcctacagagtgacactcctctttaggctagaagtctgccaagactttctgtctacctcgctttatctttcccacccacctgtggggactgcccttaagaaactgttgctgtcctatgccttgatggtatggttaactttactgggaaccttaatgctgggttttgcttgaaaagccatctgttttataaagtcaaaatgatgaaaaaaaagtcaaggattttttatacccagaccattggtaccccccccccgagatattttattccataagttgcatttttatttgacagcctgtggagctcttaggaagagttatctgtcttgtgggcaatagcttttttccctttattattccacttttcagtacaaggtactgtgtgtgatagttgcaagatatctgtactaggtaatttctatgaagaacaaagtaaaactctggccttaattcacattgcttattatagttatctgtgctggcatattgtaccaatatgacttttaaaggaagtacatgcttgcatatgtttgacgtcaatataattcagtattttgtttttgccactggaggtgcatatagtggtgaacaccacagatgaagtccagctgttctgaagccggtactttgcagtcaccatcagtcaaggaatgccagtggcttcatacagttggtgaggtgttcactatgagccaaagcagagtgggagcagggtgtaggctgtagtcaggactcatctgaaggtgagcagagacatgaaggtggcaaggggcctgtctctactccaaagacagagcatgaggttttcaggtaaaagttgtaaccagagaagtcatggttttggagaccaggcagcctttctcagccacgatttctttgcctgtgggtaccaaacttggatcattctaaatacgagaaatgagatccttatgtacagcagatactttagggcagggatctacatcctctgtaaagagctggagtgaatccttaaacttcatgtgccgaaaacttagtgtagccatagcttggttctgttcagtagagctcgttcctggataccagccttgggacttgatagagttttcacgtattacaagattttctgcatttgcattattttttaatgacctaaaactaaaaatttggttaaccctttgggctgtatagaaatgtggtaggccaagaatggttatttctgggcccctgtttggagtctagataccataggaagctgtatgaagacttgggtttgctctgagtgagataagccatgggaggtctggagcagaaatggggagggatataatttgttccacctgcattgtggagattaggctacaggaggccctggaagaagcagggatagcttcagaagttagatgtgttgtagtcagttgctagcctcggaaaatttgaggatggtacagatgaattctcccaagagcccctttggagcacagtgaaagctcttgtgtttgaaatgcaaaagttttgttccttcagaacctttcttcaaactttttaagattgaagttgttttagaaagatgaggagaggagcgttttgctctgtgaagctggatgggacccagcaaaggcaggtgtgatctcagccctagtcacagtggtcataagagaaataaggccatgtttacatttccaggacttgtggaagtcaaaccataaagtgtttttcttatggccagtgaactcactcttaatgtatatatgtatgtttttatttattcatttttgatggggtcagaggtctaacctagccctgtgttttttctaataacattaataacataaattggcaagctagaggcaagttattagtattgagagttcccttctattaatcttgtaggttgtatgatttgtagtctctgtagagatagggtacttttaatctatacctacatacctttaaatacagctatctgggggcagggtaggagtgaatatgctcaaatgatgaaatggcccacagttttacaaagaaacaagactgtttcttcgtatttaatgataatttttcttatatagatgtcttgccttttatttttacatgctaaaatattacactttggtaacttctaaacttccagaataataatgtttttcctaacctgctcaccactattggctgttttgcctggatatgtggtgatgtgccagaagccttttggttagtgaaacagtgctgatggtggtctttacaaaaacaaaacaaaacaaaacagggctaggaagatcttaatcagtgaagttcttaccttgtaaacatgagggacctgggttttattccaagaacctatgcttaaataaaagaaaaaaaacaataacaacaacaaacaaaaattgtacatggtaacatacttttatcatccccacactaggtagatagaagttgttggtttcctgagcctcctggccagccagcctatcctgcctactaaggttactcttaaactcgtaagaaaccctctctcaaaaaacaaagtggacaacttctgaggaatatcacctttcttctctgtatacatctgcacgcttacacccacacaaagacatagaaaaaccagacagaaattagaatgtggaaaggaaactttcacaaccgggactatcatatggaggacatttaaaatgagttcctgtcttagctttaatggttaattgatgaaactgttattgctattaaacatacacacacacatagtctctctctctctctctctctctctctcacacacacacacacagtctctctctctctctctctctctctgtcacacacacacacacacacacacacacaaacacacacacaaagaagccaagtacatagttttgtagatatggaaggatgtgcagtcatggcaggaactggtccagggaccagagcatccatgtttcatcacatcttagaatgtctggttatgcatcagaagccaggaaattttgacctctcacatacagtttcttgcttgctttttttttttttttttcttttcttaatgttaaggcttaggtgatacacccaaatacagtgttttgttttgctgtataccttaaacaaagtctctcttagcccatgctttcccacctcttgccccttaagccaagcacagctcccctgagaggacatagaacctaaaactcctctagtttcctgttagaaatctgaaacctaaaaatattatccctcttcttaaaagcctgtgtggcagagtcgatcccacacaaagaggaaagtgtgagacacagacctcacgagcctcctccaggtctccctgcatttgtttcattaggtcaagctgctttgttcagttgcagttcttatcagacttagtcataaaactgtatatgctaactttgatttgacagatggcagtaggatagttttttccaactcaaatcttctgtgcttttctcatataaacctgcttatcagagctgtctgggggtggtttaagcataggactcacaccttagaccatttcccattggctttttcatgtaaccttccagagtgtcactgtcttccaccctgagcttttctgaccttcccaccttgtaagaggaggttgtatcttaaacagctttctatctgcaggcctaccatagtatttggctcaaaagtagtaggcatacaagataaatgtgtttggtaagccacattgccggaaccttcaggttctcccaggatgcaccaagtacagacttggcgtgggattagggctccatgtcccaggccctgccttctgtgtctagacgagtaccgccttcaggtcctcccacggtgctatttaaacttcttatcctgtggacatcagctcaagcttaggttccctggcatctcacttagctttgcggaatgtttacgtttctctggtctcataccatcacagcactagtttatcagcattcattgttgttgttgttattatcttgtgtcttgagtcttttcagtgtatgagcctgtctctttgtttatctgcttgcagagctaaggtatcatttgtctttcttggtgtagcaaggaatttggttcttgatcagatgggctcttagaaagccatccttgatttccctggtattttcttggagtgatggagtatctgttaacacctggaggccccatagtttgtgtttttgggggactcatttgggttctctcacattttactaggagctctgaatatcagtactctgctgagcttcctagcttggcagtaatactctcagcatattgtcaacgacaggagggctaggtgtccccagggacaagaagacttcgtattcggttcccccagaatacattttcccttcttccgcctctctccaatctgtatctttttatgagttgtgactatactgcttatctgaattctatggtcattttaaggaatcaccaaacccgacagttgtgggaagggaaggcagatttgtagccaactagtatgggactcctgacctatctgttgatgtcagaagttggggcaggtggggccgacaagaggaacctccctgttcctccagctggctaactcgtggggaccataggcttcttgggacagggctttacgtatgattactcaataaagcttgtattgaattgccttgtgttttgttttgaagtattactagtctttattatgtatgtatacttaaacatagctatttctgttctattatactttgttttaaatgtagctgattgcaacttactttgttgatttcaagatttggtcttggttcattgctagtgaaaattgatttgtgttttacatgctttcttgctgtttagttacctcagccattctgtcaggaccatgaatctggttctctcagggaattctgtactgtcttgccaaccttagatgcaggcactgggcatccactttgggagtagtccactcaatctttgggccagaaatctgtatctgcccatctgccacagctaataacctctatatgtcttctccctctctttttaaggtaacatttgccggtcacccattgcagaagcagtattcaggaaactggtaactgatgaaaaggtttcagataatgtgagtaccattcagtagtgtgacgagtccagactgaatccctttgggcagcaaggtgccagaacacttcttttctctcctgcagtggaggatagacagtgcggctacatccacctatgaagtggggaaccctcctgactatcgagggcagaactgcatgagaaaacatggcatccacatgcagcacattgcacggcaggtaccgtactggagcttgacatatgttttgtgttacagtgggccattgacagcagcgccgtttccgactggaacgtgggccggcccccagacccaagagctgtcagctgcctaagaaatcatggcattagcacagcccataaggcaagacaggtagaaaagatctcgtttaatttctactacatagaatcccattacttgagagtctgaccaccctgtgtgttgcagcatgtatttggttgggggcgactgacttgctgcctgtgtagttgtggaagatttggatggccttgggtaacgtgttaaagtacagataaaagacataagccatagtgatacaatgtttgctgtgagccagacttggctgtggtcttcattttgttaccatgtacagactctctggctagacatttctgcttatgctactagtttgttagtacacatgtctttttctactttaaagtaccttaaacatacagctctagtgccttgttaacacttgtctgtctggaagactggaattaaaaacaaaccctttagttacagacaaagatagttttgtatccatattaccagaagatggcagcctgatgtatttctttgagttccttattgaaattaagttgttcttttatgttgtttttaacaaaccgttctcaactgttgttttttttattattttttggctttttagttccaaagcactagtaaagaatactttgtacgtcagtgtcacattttccttatgttctttctctttaccaattgccacactttatgcaaataactattttagtacagttcttaacatgttatttttctaatctttcaacttaggttgtaaatggcatatggttagggtgctagctcttgaactcaaagcaatgtcttgggataggaacttttagcatggccgactggagaaccgcccctgctggtcaagtgtgctcacgtgtccccgttttcagtgtcatcagcttctccatggactgagtccatcatcaaggttgcttggccgcttctcccctcttgtgcagtggtccctcccaaagtttcactgtgcgttctaacattgtaggagtagaatcatcttgcttgtgttgttgagtcagggtcacactggcacttgaggcagtcctcgtggttcagcctcctcaggctggaattgcaggtctgccccatgtccctggcacagcagtggagtgcacgcacacgtagcatcttttccatgtcgctgctgagggttacagcagtttgccctgggcagggttaagtgctgtattgttccttttgtacctctgctggacgtgtgggtggcttctgttgtgagaatctgtgggcagtggcaccccagatggagccatctaatgggttcatagccagcatctgtacctatacctcaccaaatgcacagagcatgtcttcttgtcttttaagaagtgtatgtgtctattgcatatgccagcaagattttgctgacagaaccctgacatagctctctcttgtgaggctatgccagtgcctggcaaatactaaagtggatgctcacagtcatttattagatggaacacagggcccccaatgaagaagctagagaaagtacccaaggagctgaaggggtctgcaaccctataggaggaacaacaatatgaactaaccagtacccccagagcttgtgtctctagctgcgtggcagaggatggcctagtaggccatcaatgggaggagaggcacctggtcttgtgaagattatatgccccaaatacaggggaatgccagggccaggaagcgggagtgggtgggttggggagcagcgaaggggggggggggaaggtatagggaattttcgggatagcacttaaaatgtaaatgaagaaaatatctaattaaaaaaaagaagtgtgtgtaaaaaattgatggtatctctattttgtttggaaaattgatctgattgattgcttttgctgatttttttttaatttttttaccaatttattgacattgatatttatgtttccatactttaaaaaattttttaaaattcattcatttatttatttacaccccacattttattcccctcctagtccaccctttgactgttccacatctcatacctcctccccgcccttctgtctccatgaggatgtccccattcccaccccctcaccccacctgacctctaaactcctgggtcctccagtctcttgggggttatttgcgtcatccctgattgaacctagacctgacagtcctctgctatatatgtgttgggggcctcagatcagctggtgtatgctgccaggttggtggtctagtgtttgagagatctccagggtctaggttaattgagactgctggtcttcctacagggttgccctcctcctcagcttctttcagcttttccctaattcaactactggggtcaacagcttctgtctccattggttgagtgcaaatatctgcatctgactctttcagctgcttgttgggtctttctgagggcagtcatgctaggttcctttttgtgaacgctccatagcctcagtaatagtgtcagaccttgggtcctccccttgagctggatcccactttgggcctgttgctggaccttcttttcctcaggctcctctccctttccatctctgcagttctttcagacgagattaattatgggtcagagatgtgtctgtgggatggcaactccatccctcacttgatgccctgttttttgctggaggtgggctctacaagttccctctcccctctgtagggcatttcatctaaggtccctccctttgaatccttagagtctcacctcccaggtctctggtacattctggaggatcctgcaacctcctgtctcctgaggtccaaggttgcctgtttccattctttctgctgaccctcagggcttcagtccttttcccctcacccaacaccacatcatgattccccgccccccatgtccactttccctctcagtccctccctctgcccttgtgattgctttcttctctctccctagtaggactgaggtgtcctcacttgggcccttcagcttgttgacctttttgagttctgaggactgtatcttggttattctgtacttttttttctcttcttttttcttttggctaatatccacttattagtgagtacgtaccatgcatgtccttttgggactgagttacatcattcaggataatattttctagttccatccatttgcttgcaaaactcatgatgtcctcgttcttaatagctgagtagttatcccactgggtaaatgtgtaaatacgttttctgtatccattcttctgttgtgggatatctgggtcgtttccagcttctggctatcacaaataaggctgctatgaacatagtggaacacatgcccctgtggcatggtggggcatcttttgggtatattcctaagattggtattgctgggtgttaaggtagatctattacagtttcctgaggaacctccaaaagtttccagaacttttgacttccagagtggttgtactagtttgcaatcccactagcaatggaggagtgtgataatatctcttttttttctggctgttggttttctctctggtctccagttcatcagctttgctgtgggagaccaacgcatgctttttgaactgggacttgatgctttttaaccagttttttggaaattaggctttttctttttgtttgtttggtttttttttggagaggcagctttcatatatattggacataaaaatctttctgttctttcattccgccgagtttaattagcattattttcatgccgcctatgatgtggggctgtttgctggacagtaggtaacttaccattggcagggaagaaaaattatcccctcacataccctccctcattctcaccagaattttgagggctccatgttttgtagagaacaaaagctgttgtgaatttacaagtgaacatattctttgtattttaagtaaggtctgctaatgcctttctgggtcatttctgagcctgctctttcctcttttttggagaactgggaattacaccctaggtagagcctggtacgagcttatattgcatgtgatctgctactgagctgtttccccagcagtatttagtttttatttggacagtttcaccctagcctgagctagcctgagctagcctgagctagcctgagctagcctgagctagccttgaacatgattcctgcttctctttcataaggagctgagattacaggccagtatcactaggcctggcaagttggctttcttcttgatttttctgtgtggtgcccttttggtgctgtgtaaataatcatagttcctttgaaagatcacatttttaccactgttgctgtgaggatgctggtagatgttgtcagatacagggtctcagtaggattgcagactggcagtacctctgagtacccttctgctgctgggcctcaggatggcatggactgctcacctgcagggcaggagcaggtcacagcatagaatgtgggcttttctttactgagcgcacataacttctcattggtctcttgagctttttgctctggcaggagagggcttggtaggtcaggtggaacaacctacactgactcttattttctgggcctgtgctcctccaagcttcactgtcctgccaggcctaaccccagtttcttctccattgtcccaggcatccaggcaggtagggccctgcttttccaccatggtctttgcctggctttctttgctcttgaggaagcttctaggcccttgagattattggcgtctccatcctggtggccctttgtgcaggactcagtgtgtgtgactgctagtctctagtccttgcctactcagatgtggtctagtatcaagcactagtactcaagagtgatccacgggtcctttggaagcattcctaaattgcacatagcttcaccttacatagtgtatcctaatataacccagtacagaggcaagtaagaaaatctgtctgacaatgccatttcctgaagtgaaaattaaaaatatctgtgggctagagcgatgtcacagtggctaagagtactttctcttccataggacaagagttcacacatacaatacacacacacacacacacagagagagagagagagagagagagagagagagaatgagagaaaaataaaaacatcttaacaaaaagcctatagctataatggattttttttttaaaaatcatttatttttattttatgtgtatgagtattttacctgcaagcatgcataccacatttgtgcagtgcttgcttgcagaggctaaaagagggtgttgtgagacatcgtgtgggtgctgggaactgaacctgggtcctttgcaatagcgacacatacctttaaccactgagtcatttctctagtcctgggttttcatctttttaaattaagaaaaaattgattttgttttagttttagcagtgataaaacctcctaaatgggctgtttggaaatggacttgaatgcctcgggggttaaagagctcttgcctataatgacttgagagagaactctgggagggtccaggcagggctcactcagatcagcaggtaaggtccaccataggaactcaacgggaggtgattgttatctgtggctgtaatcctgtcatgctgctctagtgagccagggttctcagaatcttggagaagaatcttggtttctgtgttgcttttctaataataggattagtttgtcctgagtttcaactgtgcattttccatgacataaagttctagctgctcttctttctcagttatttatttatttatttaaggtatagaatagagtttattaggggtatgggaagaggagttaagagggtagtagagaaagggggagggggagagaagaaagaagagaagtagaggaggagaggccagccatgagcatgtggagagaggagagaaggggatggggacagcgggaggaagggaaaagcaagagctagagaaagcaaaatagcaagaactttctcagttattttaaaatccaaaaatcctgtgtggtttgactctgtaagtcattaagcagtacattttttaggacaaattaataataataagccaaaattaatgctaagatattcttcataacttgaaaaaataacatattttatatgatagtattggtttaatgtctttgaaataaattatgagtcttgtgtcttggggaattaaaacagtacagtcctttgtgattatttaaatgctgtgctaatagtatatggatgtgcatactaaaagctaaaatgtctcatttttaatataattaatttagtataatataatttaatataattaatttaatataataattttagtaacaagcaagtgaccatttttattactgtttggttatagtcattgttcttttaacccaggtgtttcctgggtagtcaaaatctagaaggtaaatttcttcaactacatttaagggagcttatgtcttaaactatgtagtgccagaaacaagtacatgatgaagacccccaggatcttcatgttagctttagagggacttgcactcagggagtctttaccctctgagaatcagttaatctccctcttctgaggccctgtgtctcttctctgcctcactctcctggagagtatttactcttcttcatagggtggcctcacgagaacaaagatctgttccttaattaatctgtattcccaaggcctaaggcagtcatggaatgttgaatacagtggaggtgactttatgatgccaattgagattacatcttactgagggaccaaaactaacaacaagcttttttatgtccatgttttatatttatgattatttcaaagaaccttttataaaattattttgcaataaagttttggacttgaaatataccctaaattgtgtattttgttaaaatttcagttgataagtgaacatagacttaggttaatgagttaacatataattacatgttttttttttcaactacagttttgtatggatataatcactgctttttgactttttaattgtagattacaaaagaagactttgccacattcgattatatactatgtatggatgaaagcaatctgaggtaatcatatatatttgtttttttgataaagggtttctctgtctgccctataactcactctgtagacctggccttgaactcgggtctacctatcactgcctcctgagtgctaggattagaaatgacactaccatgtccagtgtatgcagtactcatactttacacagtagctctgtcctcctctcagttgtgttctaggcccagcaagtgcccggaatgactttctagtgtttttacagtttttttttttcttcttccattttttattaggtatttagctcatttacatttccaatgctataccaaaagtcccccatacccacccacccccactcccctacccacccactccccctttttggccctggcattcccctgtactggggcacataaagtttgcgtgtccaatgggccgtctttacagtttttatggacaaggttgataacaacctacagagctattcaggagggcaggaagaccaagtgtgcttatctcagagctctgtttacaaaggtgtgtcttctaaaagacacagtgcagccagtagatgaatggccatggagaccagcgggtttggggctctaggtgtggaggtgatgcgttggtcactgattcctttactgctgtaacagatgaggagtctcttgtatcctattgctatatagttgctgcactccatagcaataagtctgacaagacagccctcactgagtttatggtttttgggctaagcaacactgatgggataggggttaagtagcaatgattgagtctgtaaaactgaggtggtctgcaggtccatacccctcatcagtaaatttactttattaccaaggaagaaattgaggaactatggacttaaatcacttccctaggctcatgggacgaatgggtgggggtatagaagagctgctgccactgtggcctgcatgctctccactaaacttacttgtccagccacacttggtcctggcctcgctcagctgtcctgtactgagtccatcttcctcctgtcttcacttgccctgaacattgtccagcctgccttgggatgtggtacacatggtcttttgagttcttttctggcatttttatggatctgaagtgttatcatttttgtcatatcaaaatacattttttttttttttttggttttttgagacagggtttctctgtgtagccctggctgtcctggaactgactttgtagactatgctggcctcgaactcagaaatccgcctgcctctgcctcccaagtgctgggattaaaggcgtgcgccacatttcacagaagaaaactttattatttgagttaaagatacacattttgagtatcagaatcagtacactgtggcatacatacagctgcaggactcccataggtctgcagagtgttctgtttcatttcaaatcaggttgcactgttgtaatttgataggatatttcataaaccctgagcagatgtccctgtttaacttaaaaccatagatcagaaatctaagttcatgtttcgattttacagagatctcaatagaaaaagtaatcaagttaaaaactgcaaagctaaaattgagctacttgggagctatgatccacagaaacagctcatcattgaagatccctattatgtaagtccctgtcaggtttggagcctctgttaagatgggcaggatgtattcggtggtattaaatgaacaggatggattctgttgtattaaatctgtgtttgggtcttcggaggacactgtcatcataaaccattgctggtttttctttttctgtccttgcagggcaatgactctgacttcgaggtggtgtaccagcaatgccttaggtgctgcaaggccttcctggagaagacttactagctggtcctaagccccaccattgagcagctcactcatcagtgctgtgcccaagggtggtggcagtccttagccccataccccacctctcttttcagctgacttactgtatatctttaaaataattgtaggtgggaattaggcatatgttcagaaggataaaagcatttgagtcagacagtttgaggtgtggctaagcattcttagactaactaaacctctgaccttgcggtgattacaaaacagtggaacaagcaaatatggaacaaaagaaaacaaaaacaaaaacaaaaacccagaaagtaagagtgacctagaaggtccatatcagcctctgagctcggcaagcctgggtcgtctggtctaagtggagtgtgtgcatgacccgcacccagtgtgccgtttgcttgccgtgtactctcttctctaggctctcgttgtgacaatagctccattcacggcagccttccagttaacactggcagtttaagctcagacacactgagggtgttgagggatttgagagaggagacgctggatgtgctgatgggcctgggactccagcaggccgtcctgggctggacagtgacaccctgtctaaaaggggaacaaaaataacaacggatgggagcagcagatggataaagttaccatcttcagtcttacttggtttttgtctactttcctgagctttgttcttgtttgagccactttgctttaaaaaaaaagtaagtgtgcttaaatactggtatgtgtttgtaagtgttctctcatgggcaatttacaaagttataggcaagcaagagtaatttttgtgtattttcagaaaagagacctcaaatttatatgaatgtatgtcagaaaggaacatgaattcaactgggaagttatcaaatacaactgagatacaaatccagtgtctgcctgcttcttactgacaagtgaacaagatacctaaatgttgcctccatgtgcctttttatttgcttgagtgtgaagtttgggctctcagcctctgtgttagaaagtaaagtgatgagatggatacacacaatgctgtgttggatggcgatgggtgtctaattgagagactccaggcactatccttatccttgggccttcttcatgtagctggtgctccctacccgcttcccagctgaaaaggtggtaactgtctgtcttagcctgtcttagctcaggtcactactggtgagctccagtcacgcagaacttgtagttagaagagccatcctgacgtctgaaaggaaggaagcccaggggcctaggaatatgcagtctcttcttggccagggctcctctctcgaaggaggaaagcttacacctgactcttccagaagaaagcagctatcccagcaccctctcacagaaaggccatgatgtcccagcaaaggcagacacttcagctgcctttttgggctcctgtggggttttggtcagagtcttcacaaaattaaaaccgaggaagggagaggacaggtagtcagccacaaggaggaaatggattaagtcaaagctctccaccctacttcagtgcttgcttcagaatctcagatttctctttcaggtcataagatcctattgttttcttgaaattttacctgtgtatcttacacgcagatccttggaatgtcaggttctgctttgttctacggctgtctcaaacagcacagtgtgtttataagggctcatgaacagagaaattaactttttacattcaatcaaattacatttgtttaaaatacagccattaaattttaaaactgttctcagggtgcttcttggtttgacattctcttacaatcaggtagagatggtagatgtggagtattgatggctaaatgagaccaagcgatcaggagtttatgttattcagtacttagtcacatctacacacacacacacgcacacgtgtatatatatagtgaacaatgtgtatgtatgtttatatatacatatattctgagccttgacattgttatttaagagtcattctgaagactgttcattattccaaacacttcatttatttccttgtatttattcacagtatttacatattgtacaatacctggaatgtacttttacatttacagaggacaagtgggactggccttggcatagccacagatacatatgccactgttttcaacacaactgaggtttttttggttttcattaaaatttttcatgcagttccaaactatactttgggattttaattgtagaatacaaaatgtcaaatcatactatatgctctatgaaaatacagtttaatgttctgcctatgtttctaaagaaatattccttgtggttccacctaatcttaaaaagaaaatacctcattttacagaacaatacatcaaatgtggaatgttgtctgtttttacaatcataagagtggcaaatctcactgacagatacactgattcactgaatgcatatttgtaaactgtcagcaacatagaaaatgcaaaggaattatggaagagtgcaaaaataaatctctgtccacaggaagtgggcatgaagatggttctgttctgttggtttccaaggcacttttcaaggttcctggtgtgtcgctcctttgcagctgaaaacaattacatttggtgtcatatagctactcaggtcttattactaattctagatgatgtggtacaagtttagcaatataacaaatttataaacaaggaacctgtatttctcttaacgttcaaagtctaggtccttcccccactcccacctgaaaacaacccagttctatagttaccatgtgctcacggtgcctaagggtgacagccaccatgcacacctgtgaataaacacttgctcaagtcaagtttggtgcttggtcttaggtgtgatggggcactttgggaatcctgtcaagagatgtagtttgtgcctcctctggggaagagtttgcttg

SEQ ID NO: 14 - DNA, 477 bp, Mus musculus
atggcagaggttgggtccaagtcagtgctgttcgtgtgtcctggtaacatttgccggtcacccattgcagaagcagtattcaggaaactggtaactgatgaaaaggtttcagataattgggccattgacagcagcgccgtttccgactggaacgtgggccggcccccagacccaagagctgtcagctgcctaagaaatcgtggcattagcacagcccataaggcaagacagattacaaaagaagactttgccacattcgattatatactatgtatggatgaaagcaatctgagagatctcaatagaaaaagtaatcaagttaaaaactgcaaagctaaaattgagctacttgggagctatgatccacagaaacagctcatcattgaagatccctattatggcaatgactctgacttcgaggtggtgtaccagcaatgccttaggtgctgcaaggccttcctggagaagccttactag

SEQ ID NO: 15 - DNA, 477 bp, Mus musculus
atggcagaggttgggtccaagtcagtgctgttcgtgtgtctcggtaacatttgccggtcacccattgcagaagcagtattcaggaaactggtaactgatgaaaaggtttcagataattggaggatagacagtgcggctacatccacctatgaagtggggaaccctcctgactatcgagggcagaactgcatgagaaaacatggcatccacatgcagcacattgcacggcagattacaaaagaagactttgccacattcgattatatactatgtatggatgaaagcaatctgagagatctcaatagaaaaagtaatcaagttaaaaactgcaaagctaaaattgagctacttgggagctatgatccacagaaacagctcatcattgaagatccctattatggcaatgactctgacttcgaggtggtgtaccagcaatgccttaggtgctgcaaggccttcctggagaagacttactag

SEQ ID NO: 16 - DNA, 1436 bp, Rattus norvegicus
gggggcggtttctgggcgccagggtctgcaccgaaacatggcagaggttgggtccaagtcagtgctgttcgtgtgtctcggtaacatttgccggtcacccattgcagaagcagtgttcagaaaattggtaactgatgaaaacgtttcagataactggaggatagacagtgcggctacatccacctatgaagtggggaacccccctgactatcgagggcagaactgcatgaaaaaacatggcatccacatgcaacacattgcacggcagattacaagagaagactttgccacattcgattatatactgtgtatggatgaaagcaatctgagagatctgaatagaaaaagtaatcaagttaaaaactgcaaagctaaaatcgagctacttgggagctatgatccacagaagcagctcattattgaagatccctattatggcaatgactctgacttcgaggtggtgtaccagcaatgccttaggtgctgcaaggccttcctggagaagactcactagctggtcctaaccaccaccactgagcaacccactcctcagtgctgtgcccaagggtggtggcagtccttagccccataccccacctctcttttcagctgacttactgtatatctttaaaataattgtaggtggaaatcaggcatttgttcagaaggataaaaacatttgaggcagacatttgaggtgtggctcagtattcttagactaacaaaagctctggcctcgccataattacaaaatagtggaacgagcaactgtggaacaaaagaaaacccaataaagtaagaatgacccacaaggtccagatcagcctttgagcccagcgagcctgggtagtctggtctaactggagtgtgagcatggccagcacccagtgtgctgtttgcttgccttacactctctattctatattgtctcttattgtgacaatatctccatccatggcagccttccatttaacactgggagagtttaaacccagacacaccgagggttcagatatttgagagaggagaacctggatgtgatgatgggcctggaactccagcaggccatcctgggctgtacagtgactccctgtctgaaagataaaaatgctaacagatgggagcaacaggttggtgaagttaacacctttaatctcacttggtttttgtgtagtttcttgagcttggttcctatttgagccactttgcttctttaaagagaaagtaagtgtgcttaaatagtggtgtgcgtttgtaagtgttctctcatcaacattttacaaagttacaggcaagcatgagtaattttagtgtattttcagaaaagggacctcaaatttatgtggatatatgtcagaaaagattgtgaattaaactgggatgttatcaaatacaactaaaaatacaaaaaaaaaaaaaaaaaaaaaaaaaaaaaa

SEQ ID NO: 17 - DNA, 16001 bp, Rattus norvegicus
  misc_feature (1)...(16001): n = A,T,C or G
aaacatttatgatcaggagtgtagaaactaatcttgagttctgaggatttgcaaaagaggatccgagcagtatgatcttggtagatgcgcccagacaaacccaggaacttcactatttctctcctctttcacaacgcccttcgcaagaggaagagttgccggggtaccgacacgccccggctgctcagacccttactcactggtgccagcggctccgctccgtgcccaagctcctcactccagtccaggaagaggacggtgttctccttggtccgcctccccgaccggacccgcccagcgggcgcacacctgacccaggtgagggaataggtgtgtgccctgtttttgccaatgagatcactggtcctatacacttgctcagaacatagctcaactgcgtctacccgcctcattcctttggaaccttgcaaatcaccccctcttcttaatcgggaaaacccaccgctagtaccgtggcgcggcgtagggtcggtggtccaactgggtaacttagcttggaagggagggtagctccgatcggaggagcccccctaggagggtctgcaagccgctctctttgagctcattggcagccctggcacgttctgcttaacactgcgccacccaccctgctctgggggtcgccccgcgttcaccctgccctcgcactttagcggtgggatcaaagcagagcccagagtgaacgctagacccggttgccaaccacaactccctagcccagccctttaggcagcttctggccccgccttgggcggagtgcgaacgtggggcggcggactgcgcatgtccgcgggcagaacctggcagtgcgcatgcgccacgtctctacgcggtttctgggcgccagggtctgcaccgaaacatggcagaggttgggtccaagtcagtgctgttcgtgtgtctcggtaaggactcgccgacttaagggtctttttctttgggaccccgaagtgctctccgtgttaagcttctaaacaagagccctgggcggagggaagaagagcagaaggccttggctgggcccgcgcctaaaaacattcatttgcctggctttttcatacgcatcccccattttctggtactcccctgaacctaaggtctgtgtagttttgtcccagattatgccccggtctccttcctgcccttccgaagcacgtgtcaccctcccaagtgcccttccaccacaaatctttgtcctactcccgtctctctcccaagcctgtaggcccttttcacttgcttctcctaagtctgagagtcgcccatcacctgctgtggtagttactttcatatctctgtgctaacactgtctaaccagaacaactttagggagaacagatttgttttgattcagtttcggagggttcagtctacttttgaatgattgcagactctgggcttgtggtgaggcatcacggtcatcagggcgtccagagcatgagtcaggctacctgctccttgtggaccggaatcacggaaggggagacatggaggtagagaacaagacaagcggttagccccaagagcaagcatcgtgatgtatttattttcttctgtgtagccccacttgtgttttttcatcacctcccagtagtttggttatatttctaatctaatggagtaaaccattgactaggtcagagtccttacgatcaaatcctctcaggctatgcctttaccgacgtcctgaaagttgttttttctaaccagtcaaggtcacaatgaagataaatcatcatccctgtcatttcctcaagctcttgtccctcaccaacgtgcttttcttattctgaaccgtctcacctgctaactggcaggtggcttcatatttgtagtatgactctctaagctggccccttctattagaacagctggagttcttgccttccccagaaccccaccccacacctctctcattctgccaaggtgagcctcagttacaggaagaccattctgtgacagcagtttcccaaacaggtttgttcgatgtcatctttgggtcccagttgctgtggttctcagccagtctctattttttttttaagttgttctttttttgttctttcatttttttattggatatttatctacatttcaaatgctatccccttctcagtttttcccaccagaaaccccctatccaatcccaccatcccctgcctctataagggtgctcccccacccatacacccacccacttcttcctgccttctgccctggaattttcctacacgggggcatccagccttcacagaaccaagtgcctcccctcccattgatgcctgacaaggccatcctcttctacatatgcagctggagctataggtcccaccatgtgtactctttggttggtggtttagttcctgggagctcggggccctgggtggggggttggggggtggggtgtgatatctggttggttgatattgttgttgttcctataggggtgcaaaccccctcagccacctcggtcctttctctaactcctccaccactggggaccctgttctcagacgaatggttggctgcgagcattctttagttctctctgagaccttggttgttttcaatccctgggacaattactcagttttgctacatgaaaaatagtttttctttttataagatgaatagtctttcaaatacttctaacacagaattttcattcttagaatttcaacattgattctcctttaagtaatatgtaattcttttcttttttcagagttcttgcctcattgcctcggttcagcaatcattggtcttttggcctttgcttttcctacagagtagcgctcctcctttttcggctagaagtctgccaagactttctgtccacctcactttatctttcccacccacctgtggggaccgccctcaagaaactgttgctgtcctttgccttgatgtgtggttaattttactgggtaccatgctgggtttttcttgaaaagccatatattttataaagacaaaataaagaaaaaaaagtcaggggttttcatccccagactgttagtcacttccttttatccccctatagagatattttatcgcataaattgcatttttaattgacagcctgttgagttcttaagaagagctatctttctagtgggcaattgctttttttttcctttatttttccactttcagtacaatgtagtgtggtgtttgcaaggtatctgtactaggtaatttctatgaagagaaaagtaaaactgtggccttatttcatgttatctgtgctggcatattacacccacatgacttttttttttatttttttatttatttttttcagagcgggggaccgaacccagggcctcgctctaccactgagctaaatccccaacccccccacatgacttttaaagggaagtacatgcttgcttatgtttgacttcaatataattcagcattttgtctttgccactggagatgcatgtggtggtgaacaccgcaggtaaagtccaactgttgtgaagctggttctctgccggtctccatcagtcaaggaacgccagtggcctcgtccagtgggtgaagtgtgcagccaaaacagagtgggagctgggtgtaatttgtagtcaggactctcttgaaggtgagcagagacaaaggtggcaagagacctgtctgtctgggctcagaaggcagagcatgaggatttcaggtaaaagttgtaaccggagaaatcactattttggagatcaggcagctttctgaaccaagatttctttgcctgtgggtaccaaacttgtttcattctaaatacctggggagaaattgggtccttatatacagcagatactgtagggcagggatctgcatcctctgtaaagagctgggatgaatccttaaacttcatgtggtgataacttagtgtagccatagcttggctctgttcagtagaactcgttcctggatgccagcattggaacttgatgcagttttcacatgtcacaaggttttctgtatttgcattatttttaatgagctaaaactaaaaatttggttaaccctttagacagcatgtggatgtggtagtccaggagtggtcatttctggaccctatttggagtctaaatatcataggaggctgtatgaggattgggtttactctgcgtgagataagccttgggaggtccagaacagaaatgagagggacataatttgttccatctgcagtgtggagattaggctgcaggaggccctgggagaagcagggatatcatttgaagtaagatgtgttatagtcagttgctagtccaggaaactttgaagatggtatagatgcgttctaccaagagaccctttggaggacagtaaaagctgttgtgtttgaaatgcacaagttttattccttcagaaccttttttcgaactagttaagattgaagttgttttagcaagatgaagaaaggagcattttgctctgtgtagctagaagggactccacaaaggcaggtgtgatctcagctttagtcatagtggttgtaagaaaacgaggctatgtttaagtttcagggacttgtggaaatgaaaacctgaggctctgtgcttttctcatggccagtgtactcactcttattaatgtgtgtatgcttttattttcatttttgatgggctcagaggtctaacctcagctgtctatctttaataacattaattttaataacaaattggtgagctagaggcagagttcccttctattgatcttgtgcgttgtatgatttgtagtctccagagataggatactttgaatctatacttacacaccttactttacatatggctacctgggggtggggcatgggtgaatatgctaaaacgatgatatgtcctgagtttttacattctaaaagaaacaagactgttacttcatagttaatgataagttttcttatacagacgtcttacattttacttttacacactaagatattacatttcggttacttctaaacttctagaataatggtgcttgtcctaaactgcccaccactgttacctcttttgtctggatatgtggtgatgtgcaaaaactataaacagagtgaatcagtgccgagggtggcccttatgaaacacatcaggactagggagatcttcattggtaaagttcttgccttgtagacatcagggacctgggttttactccaagaacctatgttaaaataaaagaaaaacaaaacaacactaacaacaacaagaattgtacatggtaacatacttttataatccctgcactaggtagatagaggttgttgggtccctgagcctcctggccagccagcctgtcctactaaggttacttaaactagtaagaaactctctcaaaaataaaggtgggcaacctctgagaaataccgcccaatattgtcccctattctccatctgcacctgcacgcttgcattcttgcacccacatgtactcatagaaaaaccaaacaaaaattagaatgtagaaagggaaaccttcataaccaggactagcatatggaggacatttaaaatgaattcctgtcttagctttaatgattaattaataagactattattgctcttaatctttctttctctctctctctctctctctctctctcacacacacacacacacacacacacacacacacacacacacacagtggagggggcgggagaggaggggaagaagccaagtacttagttttgtagatatggaaggatgcacagtcatggcaggaactggtccagggaccagagcattcatatttcatcacatcttagaatgtctggttgtgcatcagaagccaggagcttttgacctcccgcatagtttcttgcttgtttaattttttcttaatgtttgggcttatgtgataacacccaaatatggtgttttgtctttccgtgcacctcagagtctctcttatcccatgccttcccacctcttgctccttaggccaagcataactcctctaagataggacatagaacctataatttctctagtttcctgtaagaaacctgaagcctagtaatattaccccccttctcccgtaaaagcctgtgtgccagagtgagtcccatacaaggaggaaagcgtgaggcacagaccaacctccccaggctcttccaggtctccctgcattggtttcattaggacgggctgcttggttcagttagagttcttatcagatgtagttgtaaaactatatataccgacgttcatttaacaaatgggaagtgggacagtttttccagctctttgcctcttcaatctgaaaattttcatgttagataaactaattaagcctcgtgtgtttttctcgtataaacctgctcatcagagttgtctggggtgatttaaatgtgggtgaggctaggaatcagaccttagactgtttcccattggctttgtcatgtatcctcccagagtctcactgtcttctaccctgagctcttctgaactccccacctcgtaagaggaagttgtatcttaaatagctttctatctgtaggccgaccataatacttggctcaaaagtaatagccatacaaaataaatgtgtttggtagccacatggccttcaggttgttctccctgggttcactaagtaccgacttggcgtgggattagggctcctcgtcccaggccctgccttctgtgtctagactagtacaggttctgccttcaagtcctcccacagtgctgcttaacttcttatcttaggaacatcagctcaagcttaggctccctggcgtcccatttagctttgtggaatctttgtttctctggtctcataccattatggcacttgtttatcagcattctcctccttctcctcatctacctcctcccccctcctcttcatcctcctcttcctccatcatcatcatcatcatcatcatcatcatcatcatcatcatcatcttcttgtgtcttgtgtcttgtgttttctcagtgtatgagtctgcctctttgtttatctgcttgcagagctaaggtgtcatttgtccttgttggtatagtaagaatttggttcttgtccaagtgggctcttaggaagtcatccttgatttccctggtattttgatggagtatctgttaacacctggaggccccatagtttgtgtttttggggactcatttggatcctgtgtcattatcccctcacatttaactaggaactctggatatcagtactatgctgagcttcctggttaggcagtagagctctcagcatattgtcagtcaatgacaggagggtgaggtgtccccaaggacaagaagacttattatttggttccccagaataaattttctcttcttccatctccaatctatatcttttaatgagttacaatatattacttatctgaattccatggtcattctaaggaatcaccaagcctgacggagagggaaggcagatgtgtagcctctagtatggagctcctgacctatctgttgacataagaagttggggtatgtgaggcagtcaagagaaacctctccctgctcctctagctggctaactcttggggaccacagtcttcttgggacaaggctttacatatgattactcagtaaagcctgtgtggaattgcgttgtgttttgctttgaagtattactagtctttattatgtatggtatacttaaaaatgcctatgtctgttctattatactttattttaaatgaagctgactgcaactcatttcattgatttcaagacttgctcctggttcattgctagtgaatattgatttgggttttatctgcttccttgagctttagttacctcagccattctgtgaggaccatgaatctagtttgattaactgttctgtcagggaattctgtactgccttgccaatcttaggtgcgggcactgggcatccgttttgggagtggtccacccagtcttcgggccaggaatttgtacctacctttctgccacagctaacgacctctgtatgtcttcttcctctcttttttaaggtaacatttgccggtcacccattgcagaagcagtgttcagaaaattggtaactgatgaaaacgtttcagataacgtgagtaccattcagtagggtgaagagtccaggctgaatacctgtgggcatcaaggtgccacaacacttcttttctctcccgcagtggaggatagacagtgcggctacatccacctatgaagtggggaacccccctgactatcgagggcagaactgcatgaaaaaacatggcatccacatgcaacacattgcacggcaggtaccgtatggagcttgacatatgttttgtgttacagtgggccattgacagcagcgccgtttccgactggaacgtgggccggcccccagacccaagagctgtcagctgcctaagaaatcatggcattagcacagcccataaggcaagacaggtagaaaagatctcgtttaatttctactacctagaattccataacttgagagtctgaccaccctgtgttttgcagaatgtattaggtaggggtcgactaacttgctgcctgtgtagttgtggaagatttggaccttgggtaacttgttaaagtacagatcccaggtataagccgcagtgaggcagtgtggctgggagccaggcctggcggtggtcttcatatgttaccatgtgcagacagacttctgctcatgctcctagcttgttagtacatagaaatgtcttttctactttgaagtatcttaaacatacagaggggctttggtgttagcacagagggggtggtgttagcacttgtctcacctgaagactatggaattaaaacaaactcttctgttacagatgaaggtagttttatattcatattatcagaaaatggcagctgttgtatttctttgagttccttattgaaattaagttgttcattgaccatttttaattgctgatctcattaggcagaagatgaaagagtgtatatcattcgttcacttaggtcgtttcaggcttttgcatacatggtccatctgccactgtctggagaacatggacttactttgagtgaccaatcaaaactgtaagacatgtggttcttttaatttctgctcatgtatagcaaaagcaaatacaatattttacattcctaggccttgaaaaccaaagccagaaacttcctccagagcagttttaatgactagctttaggagtagcatttccgtagaatctttgtttcttttatgttgttgtttttcttttattgtctttgacttttcagttccagagcactagtaaagaattcttcgtgtgtcagtatcacattttccttatgtttgtaggttcgttctctttactgattgccacacttcatgtaaataactatttagtacagttcttaacatttttctagtctgtcaacttaggtttagtaaacggcatgtggttagggtaccagttctattgaacaaaaaaaacagtgtcttgggataggagcttttagcaaggcagactagaacagtccttgctgctcaagtgtccccattttcaatgtcctttcatcagcttctccatggactgagtctaacaccaaggttatttggccgtttctcccttctcatgcagaggtcacccccaaagtttcgctcatcattttaacattataggagtaggatcattggctctgttttgctgagtccgggtcctactctggctcttgaggcagtcctcatgggtcagcctccccagtgctgggagtccgggtgtgcaccatgtccctggcatagcggagggggccgtgcacatgcagcttcttttccatgttgctgccgagtgtcagagaagttgcgttggtcagggttaagtgctgtgttgttctactgttctacctctgctggatggtgggcagcttctgtgtgagagtatgtaggcagtggcagcacagaaggcgccatctaatgggttcatagccagtgtctgtatctatgcctcaccaaatgcacggggcatggcttcttgaatttaggaagtgcgtatgtaaaaaattgatagtatcgctacttagtttggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccgagctaccccagcattatttagttttttattttgacagtttcactctgtagcctaggctagccgtgaacatgattttcctgcttctctttcacagtaggagctgaaattccaggcctgtacacctaggcttggccagttggctttcttcttgcttttgctgtgtggtgcccttttggtgctgtgtaaataatcacagtgcctgaaagatcatagttttaccgctgttgctgtgaggatgctggtagatgttgtcagatacagcatctcagtaggattgcaggcttgtagtgcctctcagtaccctctgctgctgggcctgaagatggcatggattgcttacttgcagggtgggaacagctcagagtgtggtttctctttactgagtgcacataactctccttggtctcgtgagctttctgctcgggcaggagaggacttggtcaggtcaagtgggacagcctacactaactcttttcctcggcctgtgctcttccaaggttcactgtcccgccatgcctgggcccagcttcttctctatggtcccaggcatcagggcaggtagggcctgcttttccaccatggcctttgcctggctttctttgctcttgaggaggcttctaggcccttgacaccattggcatctctgtcctggtggctctttgtgcaggactcggtgtgtgttacagctaacctctaaccattgcgtactgggttgtggtttagtatcaagcactaatacttaagagtgatccacaggtcctttggaagcattccttttttttttttttttttttttttttcttttttcggggctggggaccgaacccaggaccttgcgcttcctaggcaaacgctctaccgctgagccaaatccccaacccttggaagcattcctaaattgcacataacttcaccttacatagtgtatcctaatataacccagtgcagaggcaagtaaaaatatctgtctgacaatgccgtttcctgacctgaaaattaaaaacacctgtgggctaaagaaatatcttggtgtcttaagactacttcctcctcttccataggaccagagtgtgtacacacaaacacacaaacacacacacacacacacacacagacacagagagagagagagagagagagagagagagagagagagagagagagagagatagacaaaaaacatcttaacagaaaacccatagctataatggatttttaagtttgaaagcgttcgtttgtttttgttttatgtgtgtgagtatcttacctgagagtatgcatagcacatttgtgcagggcttgcagaggctagaagagggtgtcaccgtctctgaaataggagttacaggtgtgaggcattatgtgggtgctgggaactgaacctgggtcctttgcaatagtaacatacctttaaccaccgagccatttttctagtcctggatttgcattttttcaaattaagaaaaaattgattttgttttagttttagcattgataaaacctcctaaatgggctgtttggaaatggacttgactaccttgggggttaaagagctcttgggtataatgacttgggagagaactctgggaggatccaagcagtactcactgggatgagcagttaaggtccacacatagggactcaactggaggtgatgtgttatctgtggctatattcctgtttgtcacactgctctagtgggccaggtattcttaggagcgtgttcatagtcttggagaagagtcttggtttctgtgtaggctctaataatgggattaatttgtcctgtgcttcactgtgcattttccatgacgtaagttctagctgctcttctttctcagttcgttttaaaatacaaaaatcctgtgtggcttgactctataaatcactaagcagtacattttttaggataaattatttataataagtccaaaactaatgctaagatattcttcataacttgaaaaaataacattttatgatagtatttgtttaatgtctttgaaataagttataagtgttgggtattggggatatcaaaacaatacagtcctttgattatttaagtgctatgctattagtatatggacgtgcatactaaaagctaaaattcctcatttttaatagagtgacaagcaagtgaccatttttattactgtttggttatagtcattgttcttttaacccaggtgtttcctgggtagtcaaaatctagaagataaacttcttcagctgcactgaagggagcttacgtgtcttaaattatgtggtgccagaatagagcacattgatgaggacctgcgggatcctccctgttagcttcagacagacttgcactcgtggatccttgtccctggtgggccttctaccttttgagaatccattagttcccctgttctgaggctctgtgtcttctctctttcttgctttccttgagagtatttattctccttcacagggatggcctcacaagaacaaagaactgttccttaattaatctggagtggttcccaaggcctaaggcatggaatgatggatgtggtgggggtgactgcatggagtcatttcagattacatcttacagagggaccaaaactaacaacaacaaactttacgtccatgttttatatttctgattatttcaaagaaccttttacaaaactattttgtaataaagttttggaaatacaatgattttgttttaacttcaaatgtactctaaattgtgtgttttttttttttttaattttagtggataagtgaacatagaatttagttaatgagttaacatgtaattacatggttttttcagctataatgttgtatggatgtaatcactgttttttgatttcttaattgtagattacaagagaagactttgccacattcgattatatactgtgtatggatgaaagcaatctgaggtaatcatatatttttgtttttttgataaagggtttccctgtgtagccctgtctgccttgtaactcactttgtagatttgaccttgaactcaggtatacttatccctgcttcctgagtgttaggttaaaaggtaagcactgccatgtccagttcatgcagtaatcatactttacacagtatttctttcctcctctcagttgtgttataggcccaacaagttgtccagaatgacttgctagtgtcttacagtttttgtgtacaaggttgattacaatctacagagtgttcaggagggcaggaggatcaaggtatggttatctcagatctttacagaagtgtgtcttctgagagaccgtccagccactacaggaatggacagggagagcagctggttcggggctccaggtgtggaggtgattgatgccttggtcactggttcttttgctcctgtcacagatgaggaggcgcttgtatcctatttccgtatagttgctgctctgtagagtaagtgacaagacagaccgcactgggtttatggtttttggctaaccagcacggatgggctcagggttaagatgtggcctgcaggtccacatccctcatcaataaatttgttttattatcaaagaagaaatcaaggaaccatgaacttaaattacttccctaggttcataggatggatgggtgggctatagggagcataaaaccatagctgctgccactgtggcctgctttccactaaacttgcccaaccacacttggtcctgacctcactcagctctcctacactgagtccatcttccctgccttccttgccctgaacgttgtccaaactgtttctttgtcctgggatatggtatacatggtcttttgagttcttttctgccattttatgtatctgaaatgttatcatttttattgtatcaaaatacatttcacagaggaaaactttattatataaattaaagatacacattttgggtgtcagaatcagtgcactgtggcatacagctgcagaagtccataggtctgcagagtattctgtttcattccaaatgaggttgtactgttttgataggatatttcataaaccctgagcagatgtccctgtttaacttaaaaccatagatcagaaatctaagttcatgtttcgattttccagagatctgaatagaaaaagtaatcaagttaaaaactgcaaagctaaaatcgagctacttgggagctatgatccacagaagcagctcattattgaagatccctattatgtaagtacatttcaggtttgcagcctctgttaagatgagcaggatgtatttattctgtggaattaaatgagcaggatgtattctgttgtattaaatgacaggtttgggtcttctgaggacactgtcatcataaaccattgctagtttttctttttgtgtccttgcagggcaatgactctgacttcgaggtggtgtaccagcaatgccttaggtgctgcaaggccttcctggagaagactcactagctggtcctaaccaccaccactgagcaacccactcctcagtgctgtgcccaagggtggtggcagtccttagccccataccccacctctcttttcagctgacttactgtatatctttaaaataattgtaggtggaaatcaggcatttgttcagaaggataaaaacatttgaggcagacatttgaggtgtggctcagtattcttagactaacaaaagctctggcctcgccataattacaaaatagtggaacgagcaactgtggaacaaaagaaaacccaataaagtaagaatgacccacaaggtccagatcagcctttgagcccagcgagcctgggtagtctggtctaactggagtgtgagcatggccagcacccagtgtgctgtttgcttgccttacactctctattctatattgtctcttattgtgacaatatctccatccatggcagccttccatttaacactgggagagtttaaacccagacacaccgagggttcagatatttgagagaggagaacctggatgtgatgatgggcctggaactccnnnnnnnnnnnnnnnnnnnnnnnnnnnnnnnnnnnnnnnnnnnnnnnnnnggcgccagggtctgcaccgaaacatagcagaggttgggtccaagtcagtgctgttcgtgtgtctcggtaacatttgccggtcacccattgcagaagcagtgttcagaaaattggtaactgatgaaaacgtttcagataactgggccattgacagcagcgccgtttccgactggaacgtgggccggcccccagactcaagagctgtcagctgcctaagaaatcatggcattagcacagcccataaggcaagacagattacaagagaagactttgccacattcgattatatactgtgtatggatgaaagcaatctgagagatctgaatagaaaaagtaatcaagttaaaaactgcaaagctaaaatcgagctacttgggagctatgatccacagaagcagctcattattgaagatccctattatggcaatgactctgacttcgaggtggtgtaccagcaatgccttaggtgctgcaaggccttcctggagaagactcactagctggtcctaacc

SEQ ID NO: 18 - DNA, 459 bp, Rattus norvegicus
atggcagccttccatttaacactgggagagtttaaacccagacacaccgaggaagcagtgttcagaaaattggtaactgatgaaaacgtttcagataactgggccattgacagcagcgccgtttccgactggaacgtgggccggcccccagactcaagagctgtcagctgcctaagaaatcatggcattagcacagcccataaggcaagacagattacaagagaagactttgccacattcgattatatactgtgtatggatgaaagcaatctgagagatctgaatagaaaaagtaatcaagttaaaaactgcaaagctaaaatcgagctacttgggagctatgatccacagaagcagctcattattgaagatccctattatggcaatgactctgacttcgaggtggtgtaccagcaatgccttaggtgctgcaaggccttcctggagaagactcactag

SEQ ID NO: 19 - DNA, 20 bp, Artificial Sequence
  Oligomeric Compound
cgtgtgtctgtgctagtccc

SEQ ID NO: 20 - DNA, 20 bp, Artificial Sequence
  Oligomeric Compound
ggcaacgtgaacaggtccaa

SEQ ID NO: 21 - DNA, 18 bp, Artificial Sequence
  Oligomeric Compound
gcccattgctggacatgc

SEQ ID NO: 22 - DNA, 20 bp, Artificial Sequence
  Oligomeric Compound
agcccattgctggacatgca

SEQ ID NO: 23 - DNA, 20 bp, Artificial Sequence
  Oligomeric Compound
ttgtcccagtcccaggcctc

SEQ ID NO: 24 - DNA, 20 bp, Artificial Sequence
  Oligomeric Compound
ctttccgttggacccctggg

SEQ ID NO: 25 - DNA, 20 bp, Artificial Sequence
  Oligomeric Compound
gtgcgcgcgagcccgaaatc

SEQ ID NO: 26 - DNA, 20 bp, Artificial Sequence
  Oligomeric Compound
atccaagtgctactgtagta

SEQ ID NO: 27 - DNA, 20 bp, Artificial Sequence
  Oligomeric Compound
  misc_feature (1)...(20): n = A,T,C or G
nnnnnnnnnnnnnnnnnnnn

SEQ ID NO: 28 - DNA, 20 bp, Artificial Sequence
  Oligomeric Compound
gccctccatgctggcacagg

SEQ ID NO: 29 - DNA, 20 bp, Artificial Sequence
  Oligomeric Compound
agcaaaagatcaatccgtta

SEQ ID NO: 30 - DNA, 20 bp, Artificial Sequence
  Oligomeric Compound
tacagaaggctgggccttga

SEQ ID NO: 31 - DNA, 20 bp, Artificial Sequence
  Oligomeric Compound
atgcattctgcccccaagga

SEQ ID NO: 32 - DNA, 21 bp, Artificial Sequence
  Oligomeric Compound
caacggatttggtcgtattgg

SEQ ID NO: 33 - DNA, 26 bp, Artificial Sequence
  Oligomeric Compound
ggcaacaatatccactttaccagagt

SEQ ID NO: 34 - DNA, 21 bp, Artificial Sequence
  Oligomeric Compound
cgcctggtcaccagggctgct

SEQ ID NO: 35 - DNA, 19 bp, Artificial Sequence
  Oligomeric Compound
gaaggtgaaggtcggagtc

SEQ ID NO: 36 - DNA, 20 bp, Artificial Sequence
  Oligomeric Compound
gaagatggtgatgggatttc

SEQ ID NO: 37 - DNA, 20 bp, Artificial Sequence
  Oligomeric Compound
caagcttcccgttctcagcc

SEQ ID NO: 38 - DNA, 20 bp, Artificial Sequence
  Oligomeric Compound
tggaatcatattggaacatg

SEQ ID NO: 39 - DNA, 20 bp, Artificial Sequence
  Oligomeric Compound
ggcaaattcaacggcacagt

SEQ ID NO: 40 - DNA, 20 bp, Artificial Sequence
  Oligomeric Compound
gggtctcgctcctggaagat

SEQ ID NO: 41 - DNA, 27 bp, Artificial Sequence
  Oligomeric Compound
aaggccgagaatgggaagcttgtcatc

SEQ ID NO: 42 - DNA, 23 bp, Artificial Sequence
  Oligomeric Compound
tgttctagagacagccgcatctt

SEQ ID NO: 43 - DNA, 21 bp, Artificial Sequence
  Oligomeric Compound
caccgaccttcaccatcttgt

SEQ ID NO: 44 - DNA, 24 bp, Artificial Sequence
  Oligomeric Compound
ttgtgcagtgccagcctcgtctca

SEQ ID NO: 45 - DNA, 18 bp, Artificial Sequence
  Primer
tgcggccagcctgactag

SEQ ID NO: 46 - DNA, 23 bp, Artificial Sequence
  Primer
cgtgattacacaccgactgagaa

SEQ ID NO: 47 - DNA, 20 bp, Artificial Sequence
  Probe
ccccaccctgaggtcctgca

SEQ ID NO: 48 - DNA, 21 bp, Artificial Sequence
  Primer
gggtccaagtcagtgctgttc

SEQ ID NO: 49 - DNA, 23 bp, Artificial Sequence
  Primer
cctgaatactgcttctgcaatgg

SEQ ID NO: 50 - DNA, 26 bp, Artificial Sequence
  Probe
tgtgtctcggtaacatttgccggtca

SEQ ID NO: 51 - DNA, 23 bp, Artificial Sequence
  Primer
ggcctcgccataattacaaaata

SEQ ID NO: 52 - DNA, 24 bp, Artificial Sequence
  Primer
ggaccttgtgggtcattcttactt

SEQ ID NO: 53 - DNA, 30 bp, Artificial Sequence
  Probe
aacgagcaactgtggaacaaaagaaaaccc

SEQ ID NO: 54 - DNA, 20 bp, Artificial Sequence
  Oligomeric Compound
ctttggtaatctgccgggca

SEQ ID NO: 55 - DNA, 20 bp, Artificial Sequence
  Oligomeric Compound
actgcttctgcaatgggtga

SEQ ID NO: 56 - DNA, 20 bp, Artificial Sequence
  Oligomeric Compound
caatgacccaattctctgag

SEQ ID NO: 57 - DNA, 20 bp, Artificial Sequence
  Oligomeric Compound
tccatacatagtatataatc

SEQ ID NO: 58 - DNA, 20 bp, Artificial Sequence
  Oligomeric Compound
tttcatccatacatagtata

SEQ ID NO: 59 - DNA, 20 bp, Artificial Sequence
  Oligomeric Compound
attgctttcatccatacata

SEQ ID NO: 60 - DNA, 20 bp, Artificial Sequence
  Oligomeric Compound
agattgctttcatccataca

SEQ ID NO: 61 - DNA, 20 bp, Artificial Sequence
  Oligomeric Compound
ctcagattgctttcatccat

SEQ ID NO: 62 - DNA, 20 bp, Artificial Sequence
  Oligomeric Compound
ctctcagattgctttcatcc

SEQ ID NO: 63 - DNA, 20 bp, Artificial Sequence
  Oligomeric Compound
agcctgttccgccatcttcc

SEQ ID NO: 64 - DNA, 20 bp, Artificial Sequence
  Oligomeric Compound
gacttggtagcctgttccgc

SEQ ID NO: 65 - DNA, 20 bp, Artificial Sequence
  Oligomeric Compound
gcacggacttggtagcctgt

SEQ ID NO: 66 - DNA, 20 bp, Artificial Sequence
  Oligomeric Compound
acacaaacagcacggacttg

SEQ ID NO: 67 - DNA, 20 bp, Artificial Sequence
  Oligomeric Compound
caaatgttacccagacacac

SEQ ID NO: 68 - DNA, 20 bp, Artificial Sequence
  Oligomeric Compound
caatgggtgatcgacaaatg

SEQ ID NO: 69 - DNA, 20 bp, Artificial Sequence
  Oligomeric Compound
tgaaaactgcttctgcaatg

SEQ ID NO: 70 - DNA, 20 bp, Artificial Sequence
  Oligomeric Compound
tcggttacaagtttcctgaa

SEQ ID NO: 71 - DNA, 20 bp, Artificial Sequence
  Oligomeric Compound
ccaattctctgagatgtttt

SEQ ID NO: 72 - DNA, 20 bp, Artificial Sequence
  Oligomeric Compound
gttgccgcgctgtctaccct

SEQ ID NO: 73 - DNA, 20 bp, Artificial Sequence
  Oligomeric Compound
atgacccaccggaagttgcc

SEQ ID NO: 74 - DNA, 20 bp, Artificial Sequence
  Oligomeric Compound
tccagtcagaaacagcaccg

SEQ ID NO: 75 - DNA, 20 bp, Artificial Sequence
  Oligomeric Compound
taggcagctcacagctcttg

SEQ ID NO: 76 - DNA, 20 bp, Artificial Sequence
  Oligomeric Compound
ccatgatttcttaggcagct

SEQ ID NO: 77 - DNA, 20 bp, Artificial Sequence
  Oligomeric Compound
tttatgggctgtgtgaatgc

SEQ ID NO: 78 - DNA, 20 bp, Artificial Sequence
  Oligomeric Compound
cttgctttatgggctgtgtg

SEQ ID NO: 79 - DNA, 20 bp, Artificial Sequence
  Oligomeric Compound
aatcaaatgtggcaaaatct

SEQ ID NO: 80 - DNA, 20 bp, Artificial Sequence
  Oligomeric Compound
attcaaatctctcagattgc

SEQ ID NO: 81 - DNA, 20 bp, Artificial Sequence
  Oligomeric Compound
tgcaggttttaacttgatta

SEQ ID NO: 82 - DNA, 20 bp, Artificial Sequence
  Oligomeric Compound
ggatcatagctcccaagtag

SEQ ID NO: 83 - DNA, 20 bp, Artificial Sequence
  Oligomeric Compound
gatcttcaataataagttgt

SEQ ID NO: 84 - DNA, 20 bp, Artificial Sequence
  Oligomeric Compound
ccataatagggatcttcaat

SEQ ID NO: 85 - DNA, 20 bp, Artificial Sequence
  Oligomeric Compound
agtcattcccataataggga

SEQ ID NO: 86 - DNA, 20 bp, Artificial Sequence
  Oligomeric Compound
gtcagagtcattcccataat

SEQ ID NO: 87 - DNA, 20 bp, Artificial Sequence
  Oligomeric Compound
cgtctcaaagtcagagtcat

SEQ ID NO: 88 - DNA, 20 bp, Artificial Sequence
  Oligomeric Compound
cacactgctggtacaccgtc

SEQ ID NO: 89 - DNA, 20 bp, Artificial Sequence
  Oligomeric Compound
gtgggccttctccaagaacg

SEQ ID NO: 90 - DNA, 20 bp, Artificial Sequence
  Oligomeric Compound
gaacctgcctcagtgggcct

SEQ ID NO: 91 - DNA, 20 bp, Artificial Sequence
  Oligomeric Compound
cagcagggcacgaacctgcc

SEQ ID NO: 92 - DNA, 20 bp, Artificial Sequence
  Oligomeric Compound
gggtctagtcaggctggccg

SEQ ID NO: 93 - DNA, 20 bp, Artificial Sequence
  Oligomeric Compound
tgagaaatgcaggacctcag

SEQ ID NO: 94 - DNA, 20 bp, Artificial Sequence
  Oligomeric Compound
acacaccgactgagaaatgc

SEQ ID NO: 95 - DNA, 20 bp, Artificial Sequence
  Oligomeric Compound
gggccctggaacgtgattac

SEQ ID NO: 96 - DNA, 20 bp, Artificial Sequence
  Oligomeric Compound
aacaaagagctgggctttgg

SEQ ID NO: 97 - DNA, 20 bp, Artificial Sequence
  Oligomeric Compound
ctttttaaggtaagaaacag

SEQ ID NO: 98 - DNA, 20 bp, Artificial Sequence
  Oligomeric Compound
tgaatcaaagatttttattg

SEQ ID NO: 99 - DNA, 20 bp, Artificial Sequence
  Oligomeric Compound
aaataccccataagctgtct

SEQ ID NO: 100 - DNA, 20 bp, Artificial Sequence
  Oligomeric Compound
gcttaaaataccccataagc

SEQ ID NO: 101 - DNA, 20 bp, Artificial Sequence
  Oligomeric Compound
aagaatgcttaaaatacccc

SEQ ID NO: 102 - DNA, 20 bp, Artificial Sequence
  Oligomeric Compound
caagtgaggttttccttcat

SEQ ID NO: 103 - DNA, 20 bp, Artificial Sequence
  Oligomeric Compound
tagatgttgacctgggcctt

SEQ ID NO: 104 - DNA, 20 bp, Artificial Sequence
  Oligomeric Compound
gtctcaacaggcttagatgt

SEQ ID NO: 105 - DNA, 20 bp, Artificial Sequence
  Oligomeric Compound
gactcgattatctaagtctc

SEQ ID NO: 106 - DNA, 20 bp, Artificial Sequence
  Oligomeric Compound
aacctactgaagaggtagac

SEQ ID NO: 107 - DNA, 20 bp, Artificial Sequence
  Oligomeric Compound
aagagagaggtagcactggg

SEQ ID NO: 108 - DNA, 20 bp, Artificial Sequence
  Oligomeric Compound
ctaatctagactgtgagctc

SEQ ID NO: 109 - DNA, 20 bp, Artificial Sequence
  Oligomeric Compound
aaacacttctaatctagact

SEQ ID NO: 110 - DNA, 20 bp, Artificial Sequence
  Oligomeric Compound
ctatgggtgtgtagaaatta

SEQ ID NO: 111 - DNA, 20 bp, Artificial Sequence
  Oligomeric Compound
agtgtgcactatgggtgtgt

SEQ ID NO: 112 - DNA, 20 bp, Artificial Sequence
  Oligomeric Compound
aaatgtttctctcttcccta

SEQ ID NO: 113 - DNA, 20 bp, Artificial Sequence
  Oligomeric Compound
gccaacgactgattccataa

SEQ ID NO: 114 - DNA, 20 bp, Artificial Sequence
  Oligomeric Compound
tgaaggtgccaacgactgat

SEQ ID NO: 115 - DNA, 20 bp, Artificial Sequence
  Oligomeric Compound
gaagtattgaaggtgccaac

SEQ ID NO: 116 - DNA, 20 bp, Artificial Sequence
  Oligomeric Compound
gccaatgggctgacctcctc

SEQ ID NO: 117 - DNA, 20 bp, Artificial Sequence
  Oligomeric Compound
tggttcagatgggagccaat

SEQ ID NO: 118 - DNA, 20 bp, Artificial Sequence
  Oligomeric Compound
aagtgtccttctttctggat

SEQ ID NO: 119 - DNA, 20 bp, Artificial Sequence
  Oligomeric Compound
catattcctcaactgaccat

SEQ ID NO: 120 - DNA, 20 bp, Artificial Sequence
  Oligomeric Compound
tttgggttacatgtgcatat

SEQ ID NO: 121 - DNA, 20 bp, Artificial Sequence
  Oligomeric Compound
tgatgaagaatacttattca

SEQ ID NO: 122 - DNA, 20 bp, Artificial Sequence
  Oligomeric Compound
acatctgcctatacatttat

SEQ ID NO: 123 - DNA, 20 bp, Artificial Sequence
  Oligomeric Compound
tccccagtttattttgaaat

SEQ ID NO: 124 - DNA, 20 bp, Artificial Sequence
  Oligomeric Compound
ggaagcaactcatgatctgg

SEQ ID NO: 125 - DNA, 20 bp, Artificial Sequence
  Oligomeric Compound
aatgcatgccatatagtaga

SEQ ID NO: 126 - DNA, 20 bp, Artificial Sequence
  Oligomeric Compound
ctaatgatccaggagtgaat

SEQ ID NO: 127 - DNA, 20 bp, Artificial Sequence
  Oligomeric Compound
tggtacttacattctctgag

SEQ ID NO: 128 - DNA, 20 bp, Artificial Sequence
  Oligomeric Compound
ctttggtaatctaaaattga

SEQ ID NO: 129 - DNA, 20 bp, Artificial Sequence
  Oligomeric Compound
acaggattacctcagattgc

SEQ ID NO: 130 - DNA, 20 bp, Artificial Sequence
  Oligomeric Compound
gttgaacagaaatattcttc

SEQ ID NO: 131 - DNA, 20 bp, Artificial Sequence
  Oligomeric Compound
attcaaatctctgtaaaatt

SEQ ID NO: 132 - DNA, 20 bp, Artificial Sequence
  Oligomeric Compound
ctgtctgactcaaatgcttt

SEQ ID NO: 133 - DNA, 20 bp, Artificial Sequence
  Oligomeric Compound
ggtcagaggtttagttagtc

SEQ ID NO: 134 - DNA, 20 bp, Artificial Sequence
  Oligomeric Compound
tccgtctgcggttttatgta

SEQ ID NO: 135 - DNA, 20 bp, Artificial Sequence
  Oligomeric Compound
gtggtgctctgttgaggtgt

SEQ ID NO: 136 - DNA, 20 bp, Artificial Sequence
  Oligomeric Compound
ttgcttagtctataactgac

SEQ ID NO: 137 - DNA, 20 bp, Artificial Sequence
  Oligomeric Compound
atgatggagattgcttagtc

SEQ ID NO: 138 - DNA, 20 bp, Artificial Sequence
  Oligomeric Compound
aaatatgctaaatgatggag

SEQ ID NO: 139 - DNA, 20 bp, Artificial Sequence
  Oligomeric Compound
gcttcctgtgcaccagaaat

SEQ ID NO: 140 - DNA, 20 bp, Artificial Sequence
  Oligomeric Compound
ctcacgttgcttcctgtgca

SEQ ID NO: 141 - DNA, 20 bp, Artificial Sequence
  Oligomeric Compound
actttgtaatgggagtagat

SEQ ID NO: 142 - DNA, 20 bp, Artificial Sequence
  Oligomeric Compound
tataatggtagactttgtaa

SEQ ID NO: 143 - DNA, 20 bp, Artificial Sequence
  Oligomeric Compound
tagagaatgcaagcatatca

SEQ ID NO: 144 - DNA, 20 bp, Artificial Sequence
  Oligomeric Compound
ttcaattaatagagaatgca

SEQ ID NO: 145 - DNA, 20 bp, Artificial Sequence
  Oligomeric Compound
acatatacacatgagttgta

SEQ ID NO: 146 - DNA, 20 bp, Artificial Sequence
  Oligomeric Compound
ctttgtaatgacatatacac

SEQ ID NO: 147 - DNA, 20 bp, Artificial Sequence
  Oligomeric Compound
aaactctttgtaatgacata

SEQ ID NO: 148 - DNA, 20 bp, Artificial Sequence
  Oligomeric Compound
tgcttccatgaagcaaaact

SEQ ID NO: 149 - DNA, 20 bp, Artificial Sequence
  Oligomeric Compound
gatactttcatgcttccatg

SEQ ID NO: 150 - DNA, 20 bp, Artificial Sequence
  Oligomeric Compound
aatatgtgatactttcatgc

SEQ ID NO: 151 - DNA, 20 bp, Artificial Sequence
  Oligomeric Compound
gccataaatatgtgatactt

SEQ ID NO: 152 - DNA, 20 bp, Artificial Sequence
  Oligomeric Compound
agaccctcaatttctctaat

SEQ ID NO: 153 - DNA, 20 bp, Artificial Sequence
  Oligomeric Compound
tgccatgtttcggtgcagac

SEQ ID NO: 154 - DNA, 20 bp, Artificial Sequence
  Oligomeric Compound
acctctgccatgtttcggtg

SEQ ID NO: 155 - DNA, 20 bp, Artificial Sequence
  Oligomeric Compound
tgacttggacccaacctctg

SEQ ID NO: 156 - DNA, 20 bp, Artificial Sequence
  Oligomeric Compound
acagcactgacttggaccca

SEQ ID NO: 157 - DNA, 20 bp, Artificial Sequence
  Oligomeric Compound
acgaacagcactgacttgga

SEQ ID NO: 158 - DNA, 20 bp, Artificial Sequence
  Oligomeric Compound
acacacgaacagcactgact

SEQ ID NO: 159 - DNA, 20 bp, Artificial Sequence
  Oligomeric Compound
ttaccgagacacacgaacag

SEQ ID NO: 160 - DNA, 20 bp, Artificial Sequence
  Oligomeric Compound
aatgttaccgagacacacga

SEQ ID NO: 161 - DNA, 20 bp, Artificial Sequence
  Oligomeric Compound
gcaaatgttaccgagacaca

SEQ ID NO: 162 - DNA, 20 bp, Artificial Sequence
  Oligomeric Compound
ggtgaccggcaaatgttacc

SEQ ID NO: 163 - DNA, 20 bp, Artificial Sequence
  Oligomeric Compound
tgggtgaccggcaaatgtta

SEQ ID NO: 164 - DNA, 20 bp, Artificial Sequence
  Oligomeric Compound
caatgggtgaccggcaaatg

SEQ ID NO: 165 - DNA, 20 bp, Artificial Sequence
  Oligomeric Compound
gcaatgggtgaccggcaaat

SEQ ID NO: 166 - DNA, 20 bp, Artificial Sequence
  Oligomeric Compound
tgcttctgcaatgggtgacc

SEQ ID NO: 167 - DNA, 20 bp, Artificial Sequence
  Oligomeric Compound
atactgcttctgcaatgggt

SEQ ID NO: 168 - DNA, 20 bp, Artificial Sequence
  Oligomeric Compound
gaatactgcttctgcaatgg

SEQ ID NO: 169 - DNA, 20 bp, Artificial Sequence
  Oligomeric Compound
ctgaatactgcttctgcaat

SEQ ID NO: 170 - DNA, 20 bp, Artificial Sequence
  Oligomeric Compound
ttcctgaatactgcttctgc

SEQ ID NO: 171 - DNA, 20 bp, Artificial Sequence
  Oligomeric Compound
accagtttcctgaatactgc

SEQ ID NO: 172 - DNA, 20 bp, Artificial Sequence
  Oligomeric Compound
agttaccagtttcctgaata

SEQ ID NO: 173 - DNA, 20 bp, Artificial Sequence
  Oligomeric Compound
tcatcagttaccagtttcct

SEQ ID NO: 174 - DNA, 20 bp, Artificial Sequence
  Oligomeric Compound
cttttcatcagttaccagtt

SEQ ID NO: 175 - DNA, 20 bp, Artificial Sequence
  Oligomeric Compound
aaccttttcatcagttacca

SEQ ID NO: 176 - DNA, 20 bp, Artificial Sequence
  Oligomeric Compound
ttatctgaaaccttttcatc

SEQ ID NO: 177 - DNA, 20 bp, Artificial Sequence
  Oligomeric Compound
aattatctgaaaccttttca

SEQ ID NO: 178 - DNA, 20 bp, Artificial Sequence
  Oligomeric Compound
ggcccaattatctgaaacct

SEQ ID NO: 179 - DNA, 20 bp, Artificial Sequence
  Oligomeric Compound
atggcccaattatctgaaac

SEQ ID NO: 180 - DNA, 20 bp, Artificial Sequence
  Oligomeric Compound
tgctgtcaatggcccaatta

SEQ ID NO: 181 - DNA, 20 bp, Artificial Sequence
  Oligomeric Compound
gcgctgctgtcaatggccca

SEQ ID NO: 182 - DNA, 20 bp, Artificial Sequence
  Oligomeric Compound
ggccggcccacgttccagtc

SEQ ID NO: 183 - DNA, 20 bp, Artificial Sequence
  Oligomeric Compound
gcaaagtcttcttttgtaat

SEQ ID NO: 184 - DNA, 20 bp, Artificial Sequence
  Oligomeric Compound
aatgtggcaaagtcttcttt

SEQ ID NO: 185 - DNA, 20 bp, Artificial Sequence
  Oligomeric Compound
taatcgaatgtggcaaagtc

SEQ ID NO: 186 - DNA, 20 bp, Artificial Sequence
  Oligomeric Compound
gtatataatcgaatgtggca

SEQ ID NO: 187 - DNA, 20 bp, Artificial Sequence
  Oligomeric Compound
agtatataatcgaatgtggc

SEQ ID NO: 188 - DNA, 20 bp, Artificial Sequence
  Oligomeric Compound
catacatagtatataatcga

SEQ ID NO: 189 - DNA, 20 bp, Artificial Sequence
  Oligomeric Compound
ctttcatccatacatagtat

SEQ ID NO: 190 - DNA, 20 bp, Artificial Sequence
  Oligomeric Compound
tctctcagattgctttcatc

SEQ ID NO: 191 - DNA, 20 bp, Artificial Sequence
  Oligomeric Compound
gatctctcagattgctttca

SEQ ID NO: 192 - DNA, 20 bp, Artificial Sequence
  Oligomeric Compound
attgagatctctcagattgc

SEQ ID NO: 193 - DNA, 20 bp, Artificial Sequence
  Oligomeric Compound
ttctattgagatctctcaga

SEQ ID NO: 194 - DNA, 20 bp, Artificial Sequence
  Oligomeric Compound
gcagtttttaacttgattac

SEQ ID NO: 195 - DNA, 20 bp, Artificial Sequence
  Oligomeric Compound
aatgatgagctgtttctgtg

SEQ ID NO: 196 - DNA, 20 bp, Artificial Sequence
  Oligomeric Compound
agggatcttcaatgatgagc

SEQ ID NO: 197 - DNA, 20 bp, Artificial Sequence
  Oligomeric Compound
aatagggatcttcaatgatg

SEQ ID NO: 198 - DNA, 20 bp, Artificial Sequence
  Oligomeric Compound
cctcgaagtcagagtcattg

SEQ ID NO: 199 - DNA, 20 bp, Artificial Sequence
  Oligomeric Compound
caccacctcgaagtcagagt

SEQ ID NO: 200 - DNA, 20 bp, Artificial Sequence
  Oligomeric Compound
tggtacaccacctcgaagtc

SEQ ID NO: 201 - DNA, 20 bp, Artificial Sequence
  Oligomeric Compound
attgctggtacaccacctcg

SEQ ID NO: 202 - DNA, 20 bp, Artificial Sequence
  Oligomeric Compound
acctcgaagtcagagtcatt

SEQ ID NO: 203 - DNA, 20 bp, Artificial Sequence
  Oligomeric Compound
ggacccaacctctgccatgt

SEQ ID NO: 204 - DNA, 20 bp, Artificial Sequence
  Oligomeric Compound
ctaaggcattgctggtacac

SEQ ID NO: 205 - DNA, 20 bp, Artificial Sequence
  Oligomeric Compound
agcacctaaggcattgctgg

SEQ ID NO: 206 - DNA, 20 bp, Artificial Sequence
  Oligomeric Compound
cttgcagcacctaaggcatt

SEQ ID NO: 207 - DNA, 20 bp, Artificial Sequence
  Oligomeric Compound
aaggccttgcagcacctaag

SEQ ID NO: 208 - DNA, 20 bp, Artificial Sequence
  Oligomeric Compound
ccaggaaggccttgcagcac

SEQ ID NO: 209 - DNA, 20 bp, Artificial Sequence
  Oligomeric Compound
cttctccaggaaggccttgc

SEQ ID NO: 210 - DNA, 20 bp, Artificial Sequence
  Oligomeric Compound
gtgagtcttctccaggaagg

SEQ ID NO: 211 - DNA, 20 bp, Artificial Sequence
  Oligomeric Compound
taggaccagctagtgagtct

SEQ ID NO: 212 - DNA, 20 bp, Artificial Sequence
  Oligomeric Compound
ctcagtggtggtggttagga

SEQ ID NO: 213 - DNA, 20 bp, Artificial Sequence
  Oligomeric Compound
gccaccacccttgggcacag

SEQ ID NO: 214 - DNA, 20 bp, Artificial Sequence
  Oligomeric Compound
ggctaaggactgccaccacc

SEQ ID NO: 215 - DNA, 20 bp, Artificial Sequence
  Oligomeric Compound
gatatacagtaagtcagctg

SEQ ID NO: 216 - DNA, 20 bp, Artificial Sequence
  Oligomeric Compound
acctacaattattttaaaga

SEQ ID NO: 217 - DNA, 20 bp, Artificial Sequence
  Oligomeric Compound
tgatttccacctacaattat

SEQ ID NO: 218 - DNA, 20 bp, Artificial Sequence
  Oligomeric Compound
atgcctgatttccacctaca

SEQ ID NO: 219 - DNA, 20 bp, Artificial Sequence
  Oligomeric Compound
tctgaacaaatgcctgattt

SEQ ID NO: 220 - DNA, 20 bp, Artificial Sequence
  Oligomeric Compound
aatgtctgcctcaaatgttt

SEQ ID NO: 221 - DNA, 20 bp, Artificial Sequence
  Oligomeric Compound
acctcaaatgtctgcctcaa

SEQ ID NO: 222 - DNA, 20 bp, Artificial Sequence
  Oligomeric Compound
gagccacacctcaaatgtct

SEQ ID NO: 223 - DNA, 20 bp, Artificial Sequence
  Oligomeric Compound
gtctaagaatactgagccac

SEQ ID NO: 224 - DNA, 20 bp, Artificial Sequence
  Oligomeric Compound
ttgttagtctaagaatactg

SEQ ID NO: 225 - DNA, 20 bp, Artificial Sequence
  Oligomeric Compound
tatggcgaggccagagcttt

SEQ ID NO: 226 - DNA, 20 bp, Artificial Sequence
  Oligomeric Compound
attttgtaattatggcgagg

SEQ ID NO: 227 - DNA, 20 bp, Artificial Sequence
  Oligomeric Compound
acagttgctcgttccactat

SEQ ID NO: 228 - DNA, 20 bp, Artificial Sequence
  Oligomeric Compound
tgttccacagttgctcgttc

SEQ ID NO: 229 - DNA, 20 bp, Artificial Sequence
  Oligomeric Compound
ccttgtgggtcattcttact

SEQ ID NO: 230 - DNA, 20 bp, Artificial Sequence
  Oligomeric Compound
gctgggctcaaaggctgatc

SEQ ID NO: 231 - DNA, 20 bp, Artificial Sequence
  Oligomeric Compound
ttagaccagactacccaggc

SEQ ID NO: 232 - DNA, 20 bp, Artificial Sequence
  Oligomeric Compound
ctcacactccagttagacca

SEQ ID NO: 233 - DNA, 20 bp, Artificial Sequence
  Oligomeric Compound
cactgggtgctggccatgct

SEQ ID NO: 234 - DNA, 20 bp, Artificial Sequence
  Oligomeric Compound
gtaaggcaagcaaacagcac

SEQ ID NO: 235 - DNA, 20 bp, Artificial Sequence
  Oligomeric Compound
ttgtcacaataagagacaat

SEQ ID NO: 236 - DNA, 20 bp, Artificial Sequence
  Oligomeric Compound
ggagatattgtcacaataag

SEQ ID NO: 237 - DNA, 20 bp, Artificial Sequence
  Oligomeric Compound
ccatggatggagatattgtc

SEQ ID NO: 238 - DNA, 20 bp, Artificial Sequence
  Oligomeric Compound
ggctgccatggatggagata

SEQ ID NO: 239 - DNA, 20 bp, Artificial Sequence
  Oligomeric Compound
aaatggaaggctgccatgga

SEQ ID NO: 240 - DNA, 20 bp, Artificial Sequence
  Oligomeric Compound
agtgttaaatggaaggctgc

SEQ ID NO: 241 - DNA, 20 bp, Artificial Sequence
  Oligomeric Compound
ttaaactctcccagtgttaa

SEQ ID NO: 242 - DNA, 20 bp, Artificial Sequence
  Oligomeric Compound
ctgggtttaaactctcccag

SEQ ID NO: 243 - DNA, 20 bp, Artificial Sequence
  Oligomeric Compound
ggttctcctctctcaaatat

SEQ ID NO: 244 - DNA, 20 bp, Artificial Sequence
  Oligomeric Compound
agttccaggcccatcatcac

SEQ ID NO: 245 - DNA, 20 bp, Artificial Sequence
  Oligomeric Compound
atggcctgctggagttccag

SEQ ID NO: 246 - DNA, 20 bp, Artificial Sequence
  Oligomeric Compound
tttttatctttcagacaggg

SEQ ID NO: 247 - DNA, 20 bp, Artificial Sequence
  Oligomeric Compound
cccatctgttagcattttta

SEQ ID NO: 248 - DNA, 20 bp, Artificial Sequence
  Oligomeric Compound
ctgttgctcccatctgttag

SEQ ID NO: 249 - DNA, 20 bp, Artificial Sequence
  Oligomeric Compound
ttaacttcaccaacctgttg

SEQ ID NO: 250 - DNA, 20 bp, Artificial Sequence
  Oligomeric Compound
ccaagctcaagaaactacac

SEQ ID NO: 251 - DNA, 20 bp, Artificial Sequence
  Oligomeric Compound
aagtggctcaaataggaacc

SEQ ID NO: 252 - DNA, 20 bp, Artificial Sequence
  Oligomeric Compound
ctttctctttaaagaagcaa

SEQ ID NO: 253 - DNA, 20 bp, Artificial Sequence
  Oligomeric Compound
gcacacttactttctcttta

SEQ ID NO: 254 - DNA, 20 bp, Artificial Sequence
  Oligomeric Compound
caccactatttaagcacact

SEQ ID NO: 255 - DNA, 20 bp, Artificial Sequence
  Oligomeric Compound
acaaacgcacaccactattt

SEQ ID NO: 256 - DNA, 20 bp, Artificial Sequence
  Oligomeric Compound
gttgatgagagaacacttac

SEQ ID NO: 257 - DNA, 20 bp, Artificial Sequence
  Oligomeric Compound
gtaactttgtaaaatgttga

SEQ ID NO: 258 - DNA, 20 bp, Artificial Sequence
  Oligomeric Compound
ttactcatgcttgcctgtaa

SEQ ID NO: 259 - DNA, 20 bp, Artificial Sequence
  Oligomeric Compound
gtcccttttctgaaaataca

SEQ ID NO: 260 - DNA, 20 bp, Artificial Sequence
  Oligomeric Compound
ataaatttgaggtccctttt

SEQ ID NO: 261 - DNA, 20 bp, Artificial Sequence
  Oligomeric Compound
atatccacataaatttgagg

SEQ ID NO: 262 - DNA, 20 bp, Artificial Sequence
  Oligomeric Compound
atcttttctgacatatatcc

SEQ ID NO: 263 - DNA, 20 bp, Artificial Sequence
  Oligomeric Compound
tctccagtggcaaagacaaa

SEQ ID NO: 264 - DNA, 20 bp, Artificial Sequence
  Oligomeric Compound
aagcaagaaactatgcggga

SEQ ID NO: 265 - DNA, 20 bp, Artificial Sequence
  Oligomeric Compound
catacggtacctgccgtgca

SEQ ID NO: 266 - DNA, 20 bp, Artificial Sequence
  Oligomeric Compound
caatggcccactgtaacaca

SEQ ID NO: 267 - DNA, 20 bp, Artificial Sequence
  Oligomeric Compound
gagtacatttgaagttaaaa

SEQ ID NO: 268 - DNA, 20 bp, Artificial Sequence
  Oligomeric Compound
ctcttgtaatctacaattaa

SEQ ID NO: 269 - DNA, 20 bp, Artificial Sequence
  Oligomeric Compound
tatacctgagttcaaggtca

SEQ ID NO: 270 - DNA, 20 bp, Artificial Sequence
  Oligomeric Compound
ctcttgtaatctgtcttgcc

SEQ ID NO: 271 - DNA, 20 bp, Artificial Sequence
  Oligomeric Compound
gaggtctcgacttacccgct

SEQ ID NO: 272 - DNA, 20 bp, Artificial Sequence
  Oligomeric Compound
ttctacctcgcgcgatttac

SEQ ID NO: 273 - DNA, 20 bp, Artificial Sequence
  Oligomeric Compound
ttagaatacgtcgcgttatg

SEQ ID NO: 274 - DNA, 20 bp, Artificial Sequence
  Oligomeric Compound
ccttccctgaaggttcctcc

SEQ ID NO: 275-278 - skipped (4 entries)

SEQ ID NO: 279 - DNA, 20 bp, Artificial Sequence
  Oligomeric Compound
accccgttccgcacgccccc

SEQ ID NO: 280 - DNA, 20 bp, Artificial Sequence
  Oligomeric Compound
tagcctgttccgccatcttc

SEQ ID NO: 281 - DNA, 20 bp, Artificial Sequence
  Oligomeric Compound
gctcatgggaatgccgtgcc

SEQ ID NO: 282 - DNA, 20 bp, Artificial Sequence
  Oligomeric Compound
atcttctttggtaatctgcc

SEQ ID NO: 283 - DNA, 20 bp, Artificial Sequence
  Oligomeric Compound
atagggatcttcaataataa

SEQ ID NO: 284 - DNA, 20 bp, Artificial Sequence
  Oligomeric Compound
caccgtctcaaagtcagagt

SEQ ID NO: 285 - DNA, 20 bp, Artificial Sequence
  Oligomeric Compound
ccgactgagaaatgcaggac

SEQ ID NO: 286 - DNA, 20 bp, Artificial Sequence
  Oligomeric Compound
aacaaagagctggctttggg

SEQ ID NO: 287 - DNA, 20 bp, Artificial Sequence
  Oligomeric Compound
caaacacaactgatttccat

SEQ ID NO: 288 - DNA, 20 bp, Artificial Sequence
  Oligomeric Compound
tgaatcaaacatttttattg

SEQ ID NO: 289 - DNA, 20 bp, Artificial Sequence
  Oligomeric Compound
ttgttctactatttttgtaa

SEQ ID NO: 290 - DNA, 20 bp, Artificial Sequence
  Oligomeric Compound
gtgaggttttccttcattgt

SEQ ID NO: 291 - DNA, 20 bp, Artificial Sequence
  Oligomeric Compound
actactgtcaatccacaaaa

SEQ ID NO: 292 - DNA, 20 bp, Artificial Sequence
  Oligomeric Compound
tcttccctatcttttcaata

SEQ ID NO: 293 - DNA, 20 bp, Artificial Sequence
  Oligomeric Compound
gtattgaaggtgccaacgac

SEQ ID NO: 294 - DNA, 20 bp, Artificial Sequence
  Oligomeric Compound
taagtttcagaggcaaagtg

SEQ ID NO: 295 - DNA, 20 bp, Artificial Sequence
  Oligomeric Compound
catacaagtgtccttctttc

SEQ ID NO: 296 - DNA, 20 bp, Artificial Sequence
  Oligomeric Compound
ttattttaaaaaataagcca

SEQ ID NO: 297 - DNA, 20 bp, Artificial Sequence
  Oligomeric Compound
aaataataacacttttccca

SEQ ID NO: 298 - DNA, 20 bp, Artificial Sequence
  Oligomeric Compound
aggtttagttagtctaagaa

SEQ ID NO: 299 - DNA, 20 bp, Artificial Sequence
  Oligomeric Compound
agaggtttagttagtctaag

SEQ ID NO: 300 - DNA, 20 bp, Artificial Sequence
  Oligomeric Compound
tcagaggtttagttagtcta

SEQ ID NO: 301 - DNA, 20 bp, Artificial Sequence
  Oligomeric Compound
aaggtcagaggtttagttag

SEQ ID NO: 302 - DNA, 20 bp, Artificial Sequence
  Oligomeric Compound
gcaaggtcagaggtttagtt

SEQ ID NO: 303 - DNA, 20 bp, Artificial Sequence
  Oligomeric Compound
ccgcaaggtcagaggtttag

SEQ ID NO: 304 - DNA, 20 bp, Artificial Sequence
  Oligomeric Compound
tttgttccatatttgcttgt

SEQ ID NO: 305 - DNA, 20 bp, Artificial Sequence
  Oligomeric Compound
atgggtgaccggcaaatgtt

SEQ ID NO: 306 - DNA, 20 bp, Artificial Sequence
  Oligomeric Compound
aatgggtgaccggcaaatgt

SEQ ID NO: 307 - DNA, 20 bp, Artificial Sequence
  Oligomeric Compound
tgcaatgggtgaccggcaaa

SEQ ID NO: 308 - DNA, 20 bp, Artificial Sequence
  Oligomeric Compound
ctgcaatgggtgaccggcaa

SEQ ID NO: 309 - DNA, 20 bp, Artificial Sequence
  Oligomeric Compound
tctgcaatgggtgaccggca

SEQ ID NO: 310 - DNA, 20 bp, Artificial Sequence
  Oligomeric Compound
ttctgcaatgggtgaccggc

SEQ ID NO: 311 - DNA, 20 bp, Artificial Sequence
  Oligomeric Compound
cttctgcaatgggtgaccgg

SEQ ID NO: 312 - DNA, 20 bp, Artificial Sequence
  Oligomeric Compound
gcttctgcaatgggtgaccg

SEQ ID NO: 313 - DNA, 20 bp, Artificial Sequence
  Oligomeric Compound
ctgcttctgcaatgggtgac

SEQ ID NO: 314 - DNA, 20 bp, Artificial Sequence
  Oligomeric Compound
tactgcttctgcaatgggtg

SEQ ID NO: 315 - DNA, 20 bp, Artificial Sequence
  Oligomeric Compound
aatactgcttctgcaatggg

SEQ ID NO: 316 - DNA, 20 bp, Artificial Sequence
  Oligomeric Compound
tcctgaatactgcttctgca

SEQ ID NO: 317 - DNA, 20 bp, Artificial Sequence
  Oligomeric Compound
gtttcctgaatactgcttct

SEQ ID NO: 318 - DNA, 20 bp, Artificial Sequence
  Oligomeric Compound
cagtttcctgaatactgctt

SEQ ID NO: 319 - DNA, 20 bp, Artificial Sequence
  Oligomeric Compound
taccagtttcctgaatactg

SEQ ID NO: 320 - DNA, 20 bp, Artificial Sequence
  Oligomeric Compound
gttaccagtttcctgaatac

SEQ ID NO: 321 - DNA, 20 bp, Artificial Sequence
  Oligomeric Compound
cagttaccagtttcctgaat

SEQ ID NO: 322 - DNA, 20 bp, Artificial Sequence
  Oligomeric Compound
tgtatgctcgctcctcctct

SEQ ID NO: 323 - DNA, 20 bp, Artificial Sequence
  Oligomeric Compound
atcgaatgtggcaaagtctt

SEQ ID NO: 324 - DNA, 20 bp, Artificial Sequence
  Oligomeric Compound
ccggcgcgctcgctccgtct

SEQ ID NO: 325 - DNA, 20 bp, Artificial Sequence
  Oligomeric Compound
tataatcgaatgtggcaaag

SEQ ID NO: 326 - DNA, 20 bp, Artificial Sequence
  Oligomeric Compound
tatataatcgaatgtggcaa

SEQ ID NO: 327 - DNA, 20 bp, Artificial Sequence
  Oligomeric Compound
tagtatataatcgaatgtgg

SEQ ID NO: 328 - DNA, 20 bp, Artificial Sequence
  Oligomeric Compound
atagtatataatcgaatgtg

SEQ ID NO: 329 - DNA, 20 bp, Artificial Sequence
  Oligomeric Compound
acatagtatataatcgaatg

SEQ ID NO: 330 - DNA, 20 bp, Artificial Sequence
  Oligomeric Compound
atacatagtatataatcgaa

SEQ ID NO: 331 - DNA, 20 bp, Artificial Sequence
  Oligomeric Compound
gattgctttcatccatacat

SEQ ID NO: 332 - DNA, 20 bp, Artificial Sequence
  Oligomeric Compound
cagattgctttcatccatac

SEQ ID NO: 333 - DNA, 20 bp, Artificial Sequence
  Oligomeric Compound
tctcagattgctttcatcca

SEQ ID NO: 334 - DNA, 20 bp, Artificial Sequence
  Oligomeric Compound
ttgtcgcctcccgcgtcgtg

SEQ ID NO: 335 - DNA, 20 bp, Artificial Sequence
  Oligomeric Compound
atctctcagattgctttcat

SEQ ID NO: 336 - DNA, 20 bp, Artificial Sequence
  Oligomeric Compound
agatctctcagattgctttc

SEQ ID NO: 337 - DNA, 20 bp, Artificial Sequence
  Oligomeric Compound
tgagatctctcagattgctt

SEQ ID NO: 338 - DNA, 20 bp, Artificial Sequence
  Oligomeric Compound
ctcctgccacatgtagtccg

SEQ ID NO: 339 - DNA, 20 bp, Artificial Sequence
  Oligomeric Compound
ctgctcgggcccttattttc

SEQ ID NO: 340 - DNA, 20 bp, Artificial Sequence
  Oligomeric Compound
cacctcgaagtcagagtcat

SEQ ID NO: 341 - DNA, 20 bp, Artificial Sequence
  Oligomeric Compound
accacctcgaagtcagagtc

SEQ ID NO: 342 - DNA, 20 bp, Artificial Sequence
  Oligomeric Compound
acaccacctcgaagtcagag

SEQ ID NO: 343 - DNA, 20 bp, Artificial Sequence
  Oligomeric Compound
tacaccacctcgaagtcaga

SEQ ID NO: 344 - DNA, 20 bp, Artificial Sequence
  Oligomeric Compound
gtacaccacctcgaagtcag

SEQ ID NO: 345 - DNA, 20 bp, Artificial Sequence
  Oligomeric Compound
ggtacaccacctcgaagtca

SEQ ID NO: 346 - DNA, 20 bp, Artificial Sequence
  Oligomeric Compound
ctggtacaccacctcgaagt

SEQ ID NO: 347 - DNA, 20 bp, Artificial Sequence
  Oligomeric Compound
gctggtacaccacctcgaag

SEQ ID NO: 348 - DNA, 20 bp, Artificial Sequence
  Oligomeric Compound
tgctggtacaccacctcgaa

SEQ ID NO: 349 - DNA, 20 bp, Artificial Sequence
  Oligomeric Compound
tacgaccgcgacggccgcgt

SEQ ID NO: 350 - DNA, 20 bp, Artificial Sequence
  Oligomeric Compound
ttgctggtacaccacctcga

SEQ ID NO: 351 - DNA, 20 bp, Artificial Sequence
  Oligomeric Compound
cgaaggtgttcgtgttactc

SEQ ID NO: 352 - DNA, 20 bp, Artificial Sequence
  Oligomeric Compound
gcgttggcgcgtcgtcgctg

SEQ ID NO: 353 - DNA, 20 bp, Artificial Sequence
  Oligomeric Compound
gggtggtagtggcgggtggg

SEQ ID NO: 354 - DNA, 20 bp, Artificial Sequence
  Oligomeric Compound
gtggctgggtggtgtcgtgt

SEQ ID NO: 355 - DNA, 738 bp, Mus musculus; identical to SEQ ID NO: 12

SEQ ID NO: 356 - DNA, 15 bp, Artificial Sequence
  Oligomeric Compound
aatgccatgatttct

SEQ ID NO: 357 - DNA, 15 bp, Artificial Sequence
  Oligomeric Compound
gccatgatttcttag

SEQ ID NO: 358 - DNA, 13 bp, Artificial Sequence
  Oligomeric Compound
atgccatgatttc

SEQ ID NO: 359 - DNA, 25 bp, Artificial Sequence
  Oligomeric Compound
aatgccatgatttcttaggcagctc

SEQ ID NO: 360 - DNA, 14 bp, Artificial Sequence
  Oligomeric Compound
tttcttaggcagct

SEQ ID NO: 361 - DNA, 35 bp, Artificial Sequence
  Oligomeric Compound
tgtgaatgccatgatttcttaggcagctcacagct

SEQ ID NO: 362 - DNA, 27 bp, Artificial Sequence
  Oligomeric Compound
ccatgatttcttaggcagctcacagct

SEQ ID NO: 363 - DNA, 23 bp, Artificial Sequence
  Oligomeric Compound
gatttcttaggcagctcacagct